SEQUENCE LISTING (50 sequences)

SEQ ID NO: 1 - DNA, 380 bp, Hordeum vulgare
  CDS (3)..(380): Nucleic acid sequence coding for the callose synthase protein-1 (HvCSL-1) from Hordeum vulgare
cggcacgaggatttgtttgggggcgcctgtatctggctctgagtggtctggaggctggaattcagggcagtgctaatgctactaacaataaagccttgggtgctgtgctaaatcagcagtttgtcatacagctcggcttcttcactgccctgccaatgattttagagaattctcttgaactgggttttttacctgctgtctgggattttttcacaatgcagatgaacttttcatctgtcttctacacattttcaatgggaacaaaaagccattactatggccgaacaattcttcatggtggtgcaaagtatcgggctactggccgtggctttgttgtgcagcataagagtttcgctgaaaattacaggctatatgctagg

SEQ ID NO: 2 - protein, 126 aa, Hordeum vulgare
ARGFVWGRLYLALSGLEAGIQGSANATNNKALGAVLNQQFVIQLGFFTALPMILENSLELGFLPAVWDFFTMQMNFSSVFYTFSMGTKSHYYGRTILHGGAKYRATGRGFVVQHKSFAENYRLYAR

SEQ ID NO: 3 - DNA, 426 bp, Hordeum vulgare
  CDS (2)..(424): Nucleic acid sequence coding for the callose synthase protein-2 (HvCSL-2) from Hordeum vulgare.
cggcacgaggaatatcagtgaggatatatttgcagggtttaattctactctgcgtcaagggaacataactcaccatgagtatatccaggttggtaaaggaagagatgttgggcttaatcagatcgcactatttgaaggaaaagttgcgggaggaaacggcgaacaagttcttagcagagatatatacagacttgggcaactttttgactttttcaggatgttatccttctatgtgactactgttgggttttacttctgtacgatgctaactgtactgacagtgtacatatttctctatggtaaaacctatctggctttatctggtgttggagaatcaattcaaaatagggcggatatacagggaaatgaagcattgagcatagctctgaacacccagtttcttttccagattggtgtgtttactgc

SEQ ID NO: 4 - protein, 141 aa, Hordeum vulgare
GTRNISEDIFAGFNSTLRQGNITHHEYIQVGKGRDVGLNQIALFEGKVAGGNGEQVLSRDIYRLGQLFDFFRMLSFYVTTVGFYFCTMLTVLTVYIFLYGKTYLALSGVGESIQNRADIQGNEALSIALNTQFLFQIGVFT

SEQ ID NO: 5 - DNA, 553 bp, Hordeum vulgare
  CDS (2)..(553): Nucleic acid sequence coding for the callose synthase protein-3 (HvCSL-3) from Hordeum vulgare
cggcacgaggactggacgaggttttgttgttcgccacataaaatttgctgacaattataggctctattctcgaagccactttgtgaaagcgcttgaggttgctctcctgcttattgtctacattgcttatggctacacgaagggcgggtcatcgtcctttattctgttgactatcagtagttggttcatggttatatcctggctttttgcgccatacattttcaacccttctggtttcgagtggcaaaaaactgttgaggactttgatgactggacaaattggttattttataaaggtggagttggtgtaaagggcgaaaatagttgggaatcttggtgggatgaggagcaggctcatatccagacttttaggggacgaatccttgagactatcctgagcctcagatttctcttgttccagtatggcattgtgtacaagcttaaaattactgcacataatacatctctggcaatttatggcttctcgtggattgtacttcttgttatggttctgctgttcaagcttttcaccgcaactccaagggaagtctac

SEQ ID NO: 6 - protein, 184 aa, Hordeum vulgare
GTRTGRGFVVRHIKFADNYRLYSRSHFVKALEVALLLIVYIAYGYTKGGSSSFILLTISSWFMVISWLFAPYIFNPSGFEWQKTVEDFDDWTNWLFYKGGVGVKGENSWESWWDEEQAHIQTFRGRILETILSLRFLLFQYGIVYKLKITAHNTSLAIYGFSWIVLLVMVLLFKLFTATPREVY

SEQ ID NO: 7 - DNA, 424 bp, Hordeum vulgare
  CDS (3)..(422): Nucleic acid sequence coding for the callose synthase protein-7 (HvCSL-7) from Hordeum vulgare
ccaactttgaggccaaggtggcaaatggaaatggtgaacaaactctatgtcgtgatgtttatcgactaggacacagatttgatttctacaggatgttgtctatgtacttcaccacagttggcttctacttcaatagcatggtggccgtgcttacggtgtatgtatttttatatgggcggttatatcttgttttgagtggcttagaaaagtcgattctccaagatccacggattaaaaacatcaaaccctttgaaaatgccctagccacccaatcggtgttccaacttgggatgttgctcatcctcccgatgataatggaggttggcctggagaaagggtttggcaaagccctggctgagtttataatcatgcaacttcagttagctcctatgttcttcacttttcatctcgggaccaagactca

SEQ ID NO: 8 - protein, 140 aa, Hordeum vulgare
NFEAKVANGNGEQTLCRDVYRLGHRFDFYRMLSMYFTTVGFYFNSMVAVLTVYVFLYGRLYLVLSGLEKSILQDPRIKNIKPFENALATQSVFQLGMLLILPMIMEVGLEKGFGKALAEFIIMQLQLAPMFFTFHLGTKT

SEQ ID NO: 9 - DNA, 416 bp, Zea mays
  CDS (1)..(240): Nucleic acid sequence coding for the callose synthase protein-1 (ZmCSL-1) from Zea mays
gcgttccgtaattcccgtgtcgaccccttcgtcagaaaaccaacacttctgggagttagggaacatgttttcactggatcggcctcttcgcttgcttggatcatgtctgcacaggagacaagctttgttacccttggtcagcgagtcctagctaatccgttgaaggttcggatgcattatgggcatcctgatgtatttgatcgcctctggtttttgactcggcgtggtttaagtaaggcattcaagagtgatcaatatcagtgaggacatctttgctggtttcaactgtaccttacgtggtggcaatgttagtcaccatgagtatattcaggttggtaagggccgtgatgttggcctcaatcagatatcaatgtttgaagcaaaggtttctagtggcaacggtgaacagaccctta

SEQ ID NO: 10 - protein, 80 aa, Zea mays
AFRNSRVDPFVRKPTLLGVREHVFTGSASSLAWIMSAQETSFVTLGQRVLANPLKVRMHYGHPDVFDRLWFLTRRGLSKA

SEQ ID NO: 11 - protein, 58 aa, Zea mays
SRVINISEDIFAGFNCTLRGGNVSHHEYIQVGKGRDVGLNQISMFEAKVSSGNGEQTL

SEQ ID NO: 12 - DNA, 415 bp, Zea mays
  CDS (1)..(414): Nucleic acid sequence coding for the callose synthase protein-1a (ZmCSL-1a) from Zea mays
gcgttccgtaattcccgtgtcgaccccttcgtcagaaaaccaacacttctgggagttagggaacatgttttcactggatcggcctcttcgcttgcttggatcatgtctgcacaggagacaagctttgttacccttggtcagcgagtcctagctaatccgttgaaggttcggatgcattatgggcatcctgatgtatttgatcgcctctggtttttgactcggcgtggtttaagtaaggcatcaagagtgatcaatatcagtgaggacatctttgctggtttcaactgtaccttacgtggtggcaatgttagtcaccatgagtatattcaggttggtaagggccgtgatgttggcctcaatcagatatcaatgtttgaagcaaaggtttctagtggcaacggtgaacagaccctta

SEQ ID NO: 13 - protein, 138 aa, Zea mays
AFRNSRVDPFVRKPTLLGVREHVFTGSASSLAWIMSAQETSFVTLGQRVLANPLKVRMHYGHPDVFDRLWFLTRRGLSKASRVINISEDIFAGFNCTLRGGNVSHHEYIQVGKGRDVGLNQISMFEAKVSSGNGEQTL

SEQ ID NO: 14 - DNA, 432 bp, Zea mays
  CDS (1)..(432): Nucleic acid sequence coding for the callose synthase protein-2 (ZmCSL-2)from Zea mays
gcatcttatggcagtgcatctgggaatacattggtgtatattctgctgacactttcaagttggtttcttgtgtcatcatggattcttgctccattcatttttaatccctctggttttgactggctgaagaattttaatgactttgaggattttctaaactggatatggttccggggtgggatctcagttcagtcagatcaaagctgggagaagtggtgggaggatgaaactgatcatctccggacgacaggtctatggggctgcatcttggaaatcatattagaccttcgatttttcttttttcagtatgcaattgtatatcggcttcacattgctgataatagtagaagcatccttgtctatcttctttcgtggacatgcatcctcctagcttttgtggctcttgtgacagtggcttactttcgagacaga

SEQ ID NO: 15 - protein, 144 aa, Zea mays
ASYGSASGNTLVYILLTLSSWFLVSSWILAPFIFNPSGFDWLKNFNDFEDFLNWIWFRGGISVQSDQSWEKWWEDETDHLRTTGLWGCILEIILDLRFFFFQYAIVYRLHIADNSRSILVYLLSWTCILLAFVALVTVAYFRDR

SEQ ID NO: 16 - DNA, 414 bp, Zea mays
  CDS (85)..(405): Nucleic acid sequence coding for the callose synthase protein-3 (ZmCSL-3) from Zea mays
gctgtccgtaattcccggggcgccttcctgtcgacgatggcgggggggaggcaatttattcgattaagttgcctgtgaatccaagagcttggagagggaaaacccgaaaatcaaaatcatgccataatatttactcgtggaaatgcagtgcaaactattgatatgaatcaggataactactttgaggaggcacttaaaatgagaaacctgcttgaggagttctctctgaagcgcggcaagcattaccctactattcttggtgttagggagcatgtcttcacgggaagtgtttcctccctggcctcatttatgtctaatcaggagacaagttttgtgacattaggacagcgtgttcttgctaacccgctgaaagtgagaatgcactatggtcatccagatgttttttgatagaag

SEQ ID NO: 17 - protein, 107 aa, Zea mays
ELGEGKPENQNHAIIFTRGNAVQTIDMNQDNYFEEALKMRNLLEEFSLKRGKHYPTILGVREHVFTGSVSSLASFMSNQETSFVTLGQRVLANPLKVRMHYGHPDVF

SEQ ID NO: 18 - DNA, 5310 bp, Oryza sative
  CDS (1)..(5307): Nucleic acid sequence coding for the callose synthase protein-1 (OsCSL-1) from Oryza sativa
atgacgacgccgcgggcctcccagcgccgcggcggcgcggccgcgggcggcgcctcaccggcggcggagccgtacaacatcatccccatccacgacctcctcgcggagcacccgtcgctgcggttcccggaggtgagggcggcggcggcagcgctccgggcggtgggggggctccgcccgccgccctactcggcgtggcgcgagggccaggacctcatggactggctcggcgccttcttcgggttccagcgggacaacgtgcggaaccagcgggagcatctggtgctcctcctcgccaacgcgcagatgcggctctcctccgccgacttctccgacacgctcgagccccgcatcgcgcgcaccctccgcaggaagctcctccgcaactacaccacctggtgcggcttcctcggccgccgccccaacgtgtatgtccccgacggcgacccgcgcgccgatctgctcttcgcggggctccacctgctcgtgtggggggaggccgccaatctccgcttcgtcccggagtgcctctgctacatctaccaccacatggcgctcgagcttcaccgcatcctcgagggctacatcgacacctccacgggccgccccgccaaccccgccgtgcacggcgagaacgccttccttacccgcgtcgtcacgcccatctacggcgtcatccgcgccgaggtcgagtccagccggaacggcaccgcgccccacagcgcctggcgaaactatgacgacatcaacgagtacttctggcgccgcgacgtgttcgaccgcctcggctggcccatggagcagtcgcggcagttcttccgcacgcctcctgaccgcagccgcgtgcggaaaacgggcttcgtcgaggtccgctcgttctggaacatttaccggagcttcgacaggctgtgggtgatgctggtgctctacatgcaggccgcagccatcgtggcgtgggagagtgaggggctgccgtggaggagtctgggtaatcggaacacgcaggtgcgggttctcaccatttttatcacctgggccgctctccgcttccttcaggcgctactggatattggtacacagctccggcgtgctttcagggatggccgcatgcttgctgtgcgcatggtgctcaaggccattgtggcagctggctgggttgtggcgtttgcgatcttgtacaaggaagcctggaacaacaggaacagcaattcacagattatgagattcctgtatgcagcagcagtgtttatgatcccagaggtccttgccattgtgctatttattgtgccgtgggtgcggaatgcattggagaagacaaactggaagatatgttatgctcttacatggtggtttcagagccgaagttttgttggccgcggtttgcgtgagggcacctttgataatgtcaagtattctgtcttctgggtgcttctgcttgctgtaaagtttgcgttcagctattttctccagatcaggcctctcgtgaaacctacacaggaaatatataagttgaaaaagattgattacgcttggcatgagttctttggcaagagcaaccgatttgctgtgtttgtattgtggcttcctgtggtgttgatctatctcatggatatccaaatttggtatgctatcttctcttcactgacgggtgcatttgtggggctatttgcacatctgggggagatcagggacatgaaacagctgcgtcttcggttccagttctttgcaagtgcaatgtcattcaacattatgccagaggaacagcaggtgaatgaacgcagtttcttgcccaaccggcttcgaaatttctggcagaggctacagctacgttatggcttcagtcgatcattccggaaaatcgagtcaaatcaggtggaggcacggagattcgctcttgtttggaatgagataattactaagttccgggaggaggacattgttggtgatcgcgaagttgagcttcttgagctcccacctgagctgtggaatgtgcgtgtaatccgctggccatgtttcttgctctgtaatgagctatcacttgcacttggtcaggcaaaggaggtaaaaggacctgatcgcaagctttggaggaagatctgcaagaacgattatcgtagatgtgcagtgattgaggtatatgatagtgcaaagtacttactgcttaagataatcaaggatgatactgaggatcatgggattgtgacacaattgttccatgagtttgatgaatccatgagcatggagaagttcactgtggagtacaagatgtctgtactgccaaatgtgcatgcaaagcttgttgctatattgagcttacttctgaagcctgagaaggacattaccaagattgtcaatgctctgcagactctctatgatgttctgattcgtgacttccaggctgagaaaaggagcatggaacaactgaggaatgaaggtttagcacagtcaaggcctacgaggcttctcttcgtggacactattgttctgcctgatgaagagaagaaccccaccttctataaacaagtaaggcgcatgcacacaatcctgacctcaagggattctatgatcaatgtcccaaagaaccttgaagctcgtcgaaggattgctttcttcagtaattcgttgttcatgaacataccacgggccacccaggtggagaagatgatggccttcagcgtcttgacgccatactacaatgaagaggtgttgtacagcaaggaccagctctataaggagaatgaagatggcatctcaatcctgtactatctgcaacaaatctatcctgatgaatgggagttctttgtagaacgtatgaagcgtgaggggatgtctaatatcaaggagctgtacagtgagaagcagaggctgagagatctccggcactgggtttcatacagggggcagacactatcacgtactgtgaggggaatgatgtactactatgaagctctcaagatgctgacatttcttgattctgcatctgaacatgacttacggactggatcccgggagcttgctactatgggctcatcaaggataggatcttcgagacgggaagtgggttctgatgggtcaggatattacagcaggacatcttcgtcacgtgcattgagcagggcaagcagtagtgtaagcaccttatttaaaggcagcgagtatgggactgtccttatgaaatacacttatgtggttgcatgccagatttacggtcagcagaaagctaagaatgaccctcatgcttttgagattttagagctaatgaagaattatgaagcactacgtgttgcctatgttgatgaaaagaactccaatggtggtgaaacagaatatttctctgtccttgtgaaatatgatcagcaactgcagcgggaggttgagatttatcgtgttaagttgcctggaccactgaagcttggtgaaggcaaaccagagaaccaaaatcatgcactcatcttcacaaggggtgatgctgtccaaactattgatatgaaccaagacaactattttgaagaagctctcaagatgagaaatctgctagaggagttcaatcgccattatggaattcgcaagccaaaaatccttggggttcgggaacatgttttcactggttctgtgtcttctctagcttggttcatgtctgcccaggaaacaagttttgttactctggggcagcgtgttctggcagatccactgaaggtccgaatgcattatggccatccagatgtctttgatcgtctttggttcttgggacgaggtggtatcagtaaagcatcaagagttataaacatcagtgaggatatatttgctgggttcaattgtaccctccgtgggggtaatgttacacaccatgaatacatccaggttggtaaaggaagggatgtggggctcaatcaggtttccatgtttgaagccaaggttgctagtggcaacggtgagcaaactttgagcagagacgtttatagactggggcacagattggatttctttcggatgctctctttcttttatacaaccatcgggttttatttcaacacaatgatggtggtgctaacagtctatgcatttgtatgggggcgcttttatctcgcactgagtggtcttgaggctttcatcagcagcaatactaactccacaaataatgcagcgctaggagctgtccttaatcagcagtttgtcatacaactaggcattttcactgcactgcccatgataattgaaaactcacttgaacatgggttcctcactgcagtttgggatttcataaaaatgcaattgcagtttgcatctgttttctacaccttctcgatgggaacgaagacacattattatgggcggacaattcttcatggaggtgcaaaatatcgagccactggccgtggttttgttgtggagcacaaaaaatttgcagaaaattataggctgtatgctcgtagccacttcatcaaagcaatagagcttggtgtgatattgactctttatgcttcttatggtagcagctctgggaacacattagtgtacatcctgctgacaatttccagttggtttctagttctttcgtggattcttgctccattcatttttaatccttcaggattggattggctgaagaattttaatgattttgaggatttcctaaactggatttggttccggggtggaatctcagtgaagtcagatcaaagctgggagaagtggtgggaagaagaaactgatcatcttcggacaactggtctgtttgggagcatattggaaatcatattggaccttcggtttttcttctttcaatatgcaattgtttatcggctacacattgccggtacaagcaaaagcatccttgtctaccttctttcctgggcatgtgtcctgctggcttttgtggctcttgtgacagttgcttactttcgcgacaaatattcagcaaagaagcacatacgttaccggcttgtccaggctattattgttggtgcaacggtggctgctattgttctgttgttagaattcacaaagttccaattcattgatacctttaccagccttttggcttttcttccgactggctggggaatcatatctattgctctggtattcaagccttatctgaggaggtctgagatggtctggagaagtgtggttactttggcacgcctatatgatataatgtttggagtaattgttatggcaccagtagctgtgttgtcatggctgcctggactccaggagatgcagacgaggatcctgttcaatgaagcatttagtaggggactacatatttcccaaatcattactggaaaaaaatcacatggagtttga

SEQ ID NO: 19 - protein, 1769 aa, Oryza sative
MTTPRASQRRGGAAAGGASPAAEPYNIIPIHDLLAEHPSLRFPEVRAAAAALRAVGGLRPPPYSAWREGQDLMDWLGAFFGFQRDNVRNQREHLVLLLANAQMRLSSADFSDTLEPRIARTLRRKLLRNYTTWCGFLGRRPNVYVPDGDPRADLLFAGLHLLVWGEAANLRFVPECLCYIYHHMALELHRILEGYIDTSTGRPANPAVHGENAFLTRVVTPIYGVIRAEVESSRNGTAPHSAWRNYDDINEYFWRRDVFDRLGWPMEQSRQFFRTPPDRSRVRKTGFVEVRSFWNIYRSFDRLWVMLVLYMQAAAIVAWESEGLPWRSLGNRNTQVRVLTIFITWAALRFLQALLDIGTQLRRAFRDGRMLAVRMVLKAIVAAGWVVAFAILYKEAWNNRNSNSQIMRFLYAAAVFMIPEVLAIVLFIVPWVRNALEKTNWKICYALTWWFQSRSFVGRGLREGTFDNVKYSVFWVLLLAVKFAFSYFLQIRPLVKPTQEIYKLKKIDYAWHEFFGKSNRFAVFVLWLPVVLIYLMDIQIWYAIFSSLTGAFVGLFAHLGEIRDMKQLRLRFQFFASAMSFNIMPEEQQVNERSFLPNRLRNFWQRLQLRYGFSRSFRKIESNQVEARRFALVWNEIITKFREEDIVGDREVELLELPPELWNVRVIRWPCFLLCNELSLALGQAKEVKGPDRKLWRKICKNDYRRCAVIEVYDSAKYLLLKIIKDDTEDHGIVTQLFHEFDESMSMEKFTVEYKMSVLPNVHAKLVAILSLLLKPEKDITKIVNALQTLYDVLIRDFQAEKRSMEQLRNEGLAQSRPTRLLFVDTIVLPDEEKNPTFYKQVRRMHTILTSRDSMINVPKNLEARRRIAFFSNSLFMNIPRATQVEKMMAFSVLTPYYNEEVLYSKDQLYKENEDGISILYYLQQIYPDEWEFFVERMKREGMSNIKELYSEKQRLRDLRHWVSYRGQTLSRTVRGMMYYYEALKMLTFLDSASEHDLRTGSRELATMGSSRIGSSRREVGSDGSGYYSRTSSSRALSRASSSVSTLFKGSEYGTVLMKYTYVVACQIYGQQKAKNDPHAFEILELMKNYEALRVAYVDEKNSNGGETEYFSVLVKYDQQLQREVEIYRVKLPGPLKLGEGKPENQNHALIFTRGDAVQTIDMNQDNYFEEALKMRNLLEEFNRHYGIRKPKILGVREHVFTGSVSSLAWFMSAQETSFVTLGQRVLADPLKVRMHYGHPDVFDRLWFLGRGGISKASRVINISEDIFAGFNCTLRGGNVTHHEYIQVGKGRDVGLNQVSMFEAKVASGNGEQTLSRDVYRLGHRLDFFRMLSFFYTTIGFYFNTMMVVLTVYAFVWGRFYLALSGLEAFISSNTNSTNNAALGAVLNQQFVIQLGIFTALPMIIENSLEHGFLTAVWDFIKMQLQFASVFYTFSMGTKTHYYGRTILHGGAKYRATGRGFVVEHKKFAENYRLYARSHFIKAIELGVILTLYASYGSSSGNTLVYILLTISSWFLVLSWILAPFIFNPSGLDWLKNFNDFEDFLNWIWFRGGISVKSDQSWEKWWEEETDHLRTTGLFGSILEIILDLRFFFFQYAIVYRLHIAGTSKSILVYLLSWACVLLAFVALVTVAYFRDKYSAKKHIRYRLVQAIIVGATVAAIVLLLEFTKFQFIDTFTSLLAFLPTGWGIISIALVFKPYLRRSEMVWRSVVTLARLYDIMFGVIVMAPVAVLSWLPGLQEMQTRILFNEAFSRGLHISQIITGKKSHGV

SEQ ID NO: 20 - DNA, 1749 bp, Oryza sative
  CDS (2)..(1429): Nucleic acid sequence coding for the callose synthase protein-2 (OsCSL-2) from Oryza sativa
tgtggggctcaatcaggtttccatgtttgaagccaaggttgctagtggcaacggtgagcaaactttgagcagagacgtttatagactggggcacagattggatttctttcggatgctccctttcttttatacaaccatcgggttttatttcaacacaatgatggtggtgctaacagtctatgcatttgtatgggggcgcttttatctcgcactgagtggtcttgaggctttcatcagcagcaatactaactccacaaataatgcagcgctaggagctgtccttaatcagcagtttgtcatacaactaggcattttcactgcactgcccatgataattgaaaactcacttgaacatgggttcctcactgcagtttgggatttcataaaaatgcaattgcagtttgcatctgttttctacaccttctcgatgggaacgaagacacattattatgggcggacaattcttcatggaggtgcaaaatatcgagccactggccgtggttttgttgtggagcacaaaaaatttgcagaaaattataggctgtatgctcgtagccacttcatcaaagcaatagagcttggtgtgatattgactctttatgcttcttatggtagcagctctgggaacacattagtgtacatcctgctgacaatttccagttggtttctagttctttcgtggattcttgctccattcatttttaatccttcaggattggattggctgaagaattttaatgattttgaggatttcctaaactggatttggttccggggtggaatctcagtgaagtcagatcaaagctgggagaagtggtgggaagaagaaactgatcatcttcggacaactggtctgtttgggagcatattggaaatcatattggaccttcggtttttcttctttcaatatgcaattgtttatcggctacacattgccggtacaagcaaaagcgtccttgtctaccttctttcctgggcatgtgtcctgctggcttttgtggctcttgtgacagttgcttactttcgcgacaaatattcagcaaagaagcacatacgttaccggcttgtccaggctattattgttggtgcaacggtggctgctattgttctgttgttagaattcacaaagttccaattcattgatacctttaccagccttttggcttttcttccgactggctggggaatcatatctattgctctggtattcaagccttatctgaggaggtctgagatggtctggagaagtgtggttactttggcacgcctatatgatataatgtttggagtaattgttatggcaccagtagctgtgttgtcatggctgcctggactccaggagatgcagacgaggatcctgttcaatgaagcatttagtaggggactacatatttcccaaatcattactggaaaaaaatcacatggagtttgagctggattcatcttccttttctgaaaatgacctgccttgatggtattctattggaacgctgcccttctcaaggttcatacaggcttcccagtttagattagatggatgctagttctatgtacaggactctttatcttgattcttcttaactcaatttacacatggatttctatggtagagatgatacagtcgatcgaatgggttgtcaatttggtttatattcatggttcgagatttggtagcttattagaaattttgtagcgaaacagagctgtacaaactttatcgtgaatgcacctgcttatgccgtgcgtaac

SEQ ID NO: 21 - protein, 476 aa, Oryza sative
VGLNQVSMFEAKVASGNGEQTLSRDVYRLGHRLDFFRMLPFFYTTIGFYFNTMMVVLTVYAFVWGRFYLALSGLEAFISSNTNSTNNAALGAVLNQQFVIQLGIFTALPMIIENSLEHGFLTAVWDFIKMQLQFASVFYTFSMGTKTHYYGRTILHGGAKYRATGRGFVVEHKKFAENYRLYARSHFIKAIELGVILTLYASYGSSSGNTLVYILLTISSWFLVLSWILAPFIFNPSGLDWLKNFNDFEDFLNWIWFRGGISVKSDQSWEKWWEEETDHLRTTGLFGSILEIILDLRFFFFQYAIVYRLHIAGTSKSVLVYLLSWACVLLAFVALVTVAYFRDKYSAKKHIRYRLVQAIIVGATVAAIVLLLEFTKFQFIDTFTSLLAFLPTGWGIISIALVFKPYLRRSEMVWRSVVTLARLYDIMFGVIVMAPVAVLSWLPGLQEMQTRILFNEAFSRGLHISQIITGKKSHGV

SEQ ID NO: 22 - DNA, 1498 bp, Triticum
  CDS (34)..(1218): Nucleic acid sequence coding for the callose synthase protein-1 from (TaCSL-1)Triticum aestivum.
ggacactgacatggactgaaggagtagaaatgatcctcgagaagagaagggagtgctggtgggtcagggtattatagcagggcctcttcgtcacacacactgagcagagcaaccagtggtgtgagctctttgtttaaaggtagtgagtatgggactgtccttatgaaatacacttatgtggttgcatgccaaatttatggccagcagaaagctaaaaatgatccgcatgcttatgagatattggagctaatgaagaattatgaagcacttcgtgttgcctatgttgacgaaaaacactcggctggtgcggaaccagagtacttctccgtccttgtgaagtacgaccagcagttgcagaaagaggttgaaatttatcgagtgaagttgcctgggccactgaagcttggtgaaggcaagccagagaaccagaatcatgcactcatcttcacaaggggtgatgcagttcaaactattgatatgaaccaagacaattactttgaagaggctctgaagatgagaaatctgctggaagagttcaatcgccattatggaattcgcaagccaaaaatccttggggttcgggaacatgtgttcactggctctgtatcttctctagcttggtttatgtctgcccaagaaacaagttttgtcactctggggcagcgagttctagctaacccactcaaggttagaatgcattatggccacccagatgtgtttgatcgtctttggttcttgggccgaggtggtattagtaaagcatcaagagtaatcaacatcagtgaggatatctttgctggattcaattgtaccctccgtgggggcaatgttacacaccatgagtacatccaggttggtaaaggaagggatgtggggctcaaccaggtttctatgtttgaagccaaggttgctagtggcaatggtgagcaaactctgagccgtgatgtttacagactgggccacagattggatttctttcggatgctctcgtttttttatacaaccattggattctatttcaacacaatgatggttgtgctaactgtctatgcatttgtctgggggcgattttatcttgcacttagtgggctggaggagtacatcaccaacggacaatccttcatggaggtgcaaagtatcgagctactggccgtggatttgttgtggagcacaagaaattcgccgagaactacaggctatatgcccgtagccattttctaaaagcaatagagcttggcgtgatattggttctctatgcatcttacagcagcagcgctgggaatacatttgtgtacatcctgctgacgctttccagttggtttttagtatcctcgtggattttggcccccttcatctttaatccatcaggtttggactggctaaagaattttaatgattttgaggatttcctaagctggatttggttccagggtggaatctcagtgaagtcagatcaaagctgggaaaagtggtgggaggaggaaactgatcaccttag

SEQ ID NO: 23 - protein, 395 aa, Triticum
SSRREGSAGGSGYYSRASSSHTLSRATSGVSSLFKGSEYGTVLMKYTYVVACQIYGQQKAKNDPHAYEILELMKNYEALRVAYVDEKHSAGAEPEYFSVLVKYDQQLQKEVEIYRVKLPGPLKLGEGKPENQNHALIFTRGDAVQTIDMNQDNYFEEALKMRNLLEEFNRHYGIRKPKILGVREHVFTGSVSSLAWFMSAQETSFVTLGQRVLANPLKVRMHYGHPDVFDRLWFLGRGGISKASRVINISEDIFAGFNCTLRGGNVTHHEYIQVGKGRDVGLNQVSMFEAKVASGNGEQTLSRDVYRLGHRLDFFRMLSFFYTTIGFYFNTMMVVLTVYAFVWGRFYLALSGLEEYITNGQSFMEVQSIELLAVDLLWSTRNSPRTTGYMPVAIF

SEQ ID NO: 24 - DNA, 442 bp, Triticum aestivum
  CDS (2)..(442): Nucleic acid sequence coding for the callose synthase protein-2 (TaCSL-2) from Triticum aestivum
aagggaaaccagaaaatcagaaccatgcgataatttttactcgaggcgaaggctacagaccatagacatgaatcaggagcattatatggaggagacattgaaaatgagaaacctgctgcaagagtttacgaagaaacatgatggtgtgaggtatccgacaatacttggtgtaagagaacatatattcactggcagtgtttcttcgcttgcgtggttcatgtcaaaccaagagacaagttttgtgactattggacagcgtgtacttgccaatcctttaagggttcgatttcattatggacatcctgatatctttgatcgacttttccatctcacaaggggtggtgtaagcaaagcatctaagattatcaatcttagtgaggacatatttgctggattcaattcaacgctgcgtgaaggaaacgttacacatcatgaatacatg

SEQ ID NO: 25 - protein, 147 aa, Triticum aestivum
RETRKSEPCDNFYSRRRLQTIDMNQEHYMEETLKMRNLLQEFTKKHDGVRYPTILGVREHIFTGSVSSLAWFMSNQETSFVTIGQRVLANPLRVRFHYGHPDIFDRLFHLTRGGVSKASKIINLSEDIFAGFNSTLREGNVTHHEYM

SEQ ID NO: 26 - DNA, 389 bp, Triticum aestivum
  CDS (2)..(388): Nucleic acid sequence coding for the callose synthase protein-4 (TaCSL-4) from Triticum aestivum.
ctgcaccctgcgtggtggaaatgttagccaccatgagtacatccaggttggcaaggggcgtgatgttgggcttaatcagatatcgatgtttgaagctaaggtatccagtggcaatggtgaacagacattgagtagggatatgtacagactgggtcatagaactgattttttccggatgctttctgtgttttatacaacagtgggattctacttcaacacaatgctggtggtcttgacggtttacacatttgtttgggggcgcctgtatctggctctgagtggtctggaggctggaattcagggcagtgctaatgctactaacaacaaagccttgggtgctgtgctaaatcagcagtttgtcatacagctcggattcttcactgccctgc

SEQ ID NO: 27 - protein, 129 aa, Triticum aestivum
CTLRGGNVSHHEYIQVGKGRDVGLNQISMFEAKVSSGNGEQTLSRDMYRLGHRTDFFRMLSVFYTTVGFYFNTMLVVLTVYTFVWGRLYLALSGLEAGIQGSANATNNKALGAVLNQQFVIQLGFFTAL

SEQ ID NO: 28 - DNA, 374 bp, Triticum aestivum
  CDS (2)..(373): Nucleic acid sequence coding for the callose synthase protein-5 (TaCSL-5) from Triticum aestivum.
gaggaatttacttgaagaatttcgtggtaaccatggaatacgttatcccacaattcttggtgtgcgggacgatgtgtttacgggaagtgtgtcttcgctggcatcatttatgtctaaacaggaaaccagtttcgttactttggggcaacgtgttcttgcttacctcaaggttcgaatgcactacgggcatcctgatgtctttgatcggatatttcatataaccaggggtggtattagcaaggcatcccgggtgatcaatataagtgaagatatatacgctggattcaattcaactctgcgccagggtaatatcacacaccatgaatatatccaggttggaaaaggacgtgatgttggtttaaaccagattgccc

SEQ ID NO: 29 - protein, 124 aa, Triticum aestivum
RNLLEEFRGNHGIRYPTILGVRDDVFTGSVSSLASFMSKQETSFVTLGQRVLAYLKVRMHYGHPDVFDRIFHITRGGISKASRVINISEDIYAGFNSTLRQGNITHHEYIQVGKGRDVGLNQIA

SEQ ID NO: 30 - DNA, 467 bp, Triticum aestivum
  CDS (97)..(465): Nucleic acid sequence coding for the callose synthase protein-6 (TaCSL-6) from Triticum aestivum
cggtctcgcattcccgtgcagagagttcgggagagaccgggtatgccgctcacagtgtgagtgccaggaacacacttgtttacatagtcatgatgatatctagctgtggttctcgtggtgtcctggatcatggctccatttgcatttaatccttctggctttgagtgggtagaagacgtgtattatttcgaggatttcatgacctggatctggtttccaggaggtatattttctaaggctgagcacagctgggaagtgtggtggtatgaggagcaagatcatctgcggactactggccttttgggtgagattttggagatagtgttagatctcagatacttcttttttcagtatggggttgtataccagctcaaaatcgcagacggaagcagaagtattgcggtgtatctgctttcctggatatgtgtggcagtgatcttttggggttttgtcctgatggcctatac

SEQ ID NO: 31 - protein, 123 aa, Triticum aestivum
YLAVVLVVSWIMAPFAFNPSGFEWVEDVYYFEDFMTWIWFPGGIFSKAEHSWEVWWYEEQDHLRTTGLLGEILEIVLDLRYFFFQYGVVYQLKIADGSRSIAVYLLSWICVAVIFWGFVLMAY

SEQ ID NO: 32 - DNA, 411 bp, Triticum aestivum
  CDS (2)..(409): Nucleic acid sequence coding for the callose synthase protein-7 (TaCSL-7) from Triticum aestivum
agagagcggggcctgctggtcgaagacactcggcccgtatgaaatttaccgcatcaagcttcctggcaaacccacagatattggagagggcaaacctgaaaatcaaaatcatgccataattttcaccaggggtgaagcactccaggccattgatatgaatcaggataattaccttgaagaggcatttaaaatgagaaatgtgctggaggagttcgggagtgacaaatatggaaagagcaagcccactattttaggtcttcgggagcatatttttactggaagtgtttcatcacttgcttggtttatgtcaaaccaagagaccagctttgttacgattggacagcgtgttttggccaatcctctcaaggttcggttccattatggccatcctgatatttttgatagactctt

SEQ ID NO: 33 - protein, 136 aa, Triticum aestivum
ESGACWSKTLGPYEIYRIKLPGKPTDIGEGKPENQNHAIIFTRGEALQAIDMNQDNYLEEAFKMRNVLEEFGSDKYGKSKPTILGLREHIFTGSVSSLAWFMSNQETSFVTIGQRVLANPLKVRFHYGHPDIFDRL

SEQ ID NO: 34 - DNA, 5642 bp, Arabidopsis thaliana
  CDS (1)..(5340): Nucleic acid sequence coding for the glucan synthase-like protein-5 from A.thalina (accession no. NM_116593).
atgagcctccgccaccgcaccgtcccgccgcaaaccggacggccgttggcggcggaagctgtcggaatcgaagaggagccgtacaatatcattcccgttaacaatctcctcgccgaccatccttcactccgttttcccgaggttcgtgccgccgctgctgctcttaaaaccgttggagaccttcgtcgtccgccgtatgttcaatggcgttctcactacgatctcctcgactggctcgccttgttcttcggtttccagaaagataacgttcgtaaccagcgtgagcatatggtgcttcatctcgcaaatgctcagatgcgtctctctccgccgccggataatattgattctctcgattccgcggttgttcgtcggtttcgtcggaaacttctcgctaactactctagctggtgttcgtatttggggaaaaaatcaaatatctggatctcagatcggaaccctgattcgagacgagagcttctctatgttggactctatcttctcatttggggagaggctgcgaatcttcggttcatgcctgaatgtatctgttacatcttccataacatggcctctgagctcaacaaaatcttagaggattgcctcgatgagaacaccggccaaccttacttgccttctctctcaggcgaaaacgctttcttaaccggcgtcgttaaacctatttacgatactatccaagctgagattgatgagagcaagaacggtacagttgcgcattgtaagtggaggaactacgacgatatcaatgagtacttctggactgatcggtgtttcagcaaattgaaatggccgcttgatttgggaagcaatttctttaagagtagaggcaaaagtgtagggaaaactggtttcgtggagcgcaggacgttcttctacctttacaggagttttgatcgactttgggtgatgctagctttgttccttcaagccgccattatagtagcttgggaggaaaagccagatacctcgtcggtaacaaggcagctgtggaatgctctgaaggcaagagatgttcaggtgagactattgaccgtgttcttgacatggagtggtatgcgactcttgcaggctgtgctggacgcggcttcacaatatcccctcgtttccagagagaccaaaaggcattttttcagaatgctgatgaaggttatagctgccgcagtttggattgtagctttcactgtcctctacactaacatctggaagcagaagaggcaagacaggcagtggtccaatgccgcgacgactaagatataccaattcctttacgctgtgggggccttcttggtgcccgaaatcctggctttggctttgtttattatcccatggatgagaaacttcctggaagagaccaattggaaaatattctttgctctaacttggtggtttcaaggcaaaagctttgtgggtcgaggtttgagagagggtttagtggacaacatcaagtactcgactttctggatctttgtcctagctacaaagtttacatttagttacttcctgcaggttaagccaatgattaaaccctcaaagctgctatggaacttaaaggatgtcgattatgagtggcatcagttttatggagacagcaataggttttctgtcgcattgttatggttgccagttgtgttgatatatctgatggatatccaaatttggtacgcaatctattcttcgattgttggtgctgttgttgggctgtttgatcatctgggggagatcagggacatgggacagctgaggctaaggtttcaattctttgctagtgctattcaattcaacctaatgcctgaggaacaactcctgaatgctagaggctttggtaacaagttcaaggacggcattcatagattgaagctaaggtatggatttgggaggccgtttaagaaacttgagtcgaatcaggtcgaggccaacaagtttgcgttgatctggaacgaaatcatcttagctttcagagaagaggatatagtttctgatcgtgaagtagagctactggagctgccaaagaattcctgggatgtgacggttattcgctggccgtgtttcttgttgtgcaatgagcttttgcttgcactgagccaggccagagagctgatagacgcacctgataaatggctgtggcacaaaatatgcaagaatgaatacaggcgttgtgctgtagttgaggcatatgacagcatcaaacatctattgctctcaatcatcaaagttgacactgaagaacattcgataattacggtcttctttcagataattaatcagtccattcagtcagagcagttcaccaagacctttagagtggacctgctgccaaaaatttatgaaacactgcagaaattggttgggctggtaaatgatgaggaaacagatagtgggcgggtggtgaatgttctgcagtctctttatgagattgcaactcgacagttctttatagagaagaagacaactgaacagctatctaatgaaggtttaactcctcgagacccagcctcaaagttgctgtttcaaaatgctattaggcttcctgatgcaagcaatgaagacttctaccggcaggttaggcgtttacacacgattctcacctctagggactctatgcacagcgtccctgtgaatctagaggcgagacggcggattgctttcttcagtaattcgcttttcatgaacatgcctcatgcccctcaggttgagaaaatgatggcgttcagtgttctgactccatattacagtgaggaagttgtatacagcaaagaacagctccgaaatgagactgaggatgggatttccaccctatactacctgcagacaatttatgctgatgaatggaaaaatttcaaggaacggatgcatagggaaggaatcaagacagatagtgagttgtggacaaccaagctgagagacctcaggctttgggcttcctacagaggtcagacattggcacgtacagttcgtgggatgatgtactactaccgggctcttaagatgctcgcttttcttgactctgcgtctgaaatggacattcgggagggtgctcaggagcttggttcagtgaggaatttgcagggagaactgggtggtcaatctgatgggtttgtctctgaaaacgaccgatcttccttaagcagagcaagtagttccgtgagtacgctgtataaaggccatgagtatgggactgcattgatgaaattcacatatgttgtggcgtgtcagatctacgggtctcaaaaagcaaagaaagagcctcaggcagaggaaattctgtatctgatgaagcagaacgaagctctccgtattgcatatgtggatgaggtgcctgcgggaagaggagagactgattattactccgttctggtgaaatacgatcaccagttggagaaggaagtggaaatattccgtgtgaagctacctggtccagtgaagctgggcgagggaaagccagagaaccagaatcatgcaatgatctttacccgtggtgatgctgttcagaccattgatatgaaccaagacagttattttgaggaagctctcaagatgagaaatttgctccaggagtacaaccattatcatggtatcagaaaaccaactattcttggtgtcagggagcatatcttcacgggatcagtctcgtcactggcgtggttcatgtctgctcaggagacaagttttgtcactcttggtcagcgtgttcttgcaaacccactgaaggtcagaatgcattatggccaccctgatgtatttgacagattctggttcttgagtcgaggcggcatcagtaaggcttccagagttataaatatcagtgaggacatctttgccgggtttaactgcacgttaagggggggaaacgtcacccaccacgagtacattcaggtcgggaagggacgggatgttggattgaatcagatatcaatgtttgaggctaaggtagccagtgggaacggagagcaggttctcagccgagatgtgtaccggctcgggcacaggcttgatttcttcagaatgttatcatttttctacacaactgtagggtttttcttcaacacaatgatggtcattcttactgtttacgctttcctctggggacgggtttatctggctctcagcggggttgagaagtccgctctagcagacagtacggacaccaacgccgcgcttggggtgatcctgaaccagcagttcatcattcagctcggtctgttcactgccctgccaatgattgttgaatggtctctcgaggagggtttccttctagcgatatggaatttcattcgaatgcagattcagctttcagctgtcttctacacattctcaatggggaccagagctcactatttcggtcgaactattctccatggtggggccaagtatagagccactggacgtggatttgttgtcgagcacaagggattcactgagaactaccgactgtatgcacgcagtcactttgtgaaggccatcgagcttgggctgatcctcatagtctacgcttcgcacagtccgattgccaaagactcgttgatttacatagccatgactatcaccagctggtttcttgtgatttcatggataatggccccatttgtgtttaacccatcaggattcgactggcttaagacagtctatgactttgaagacttcatgaactggatctggtaccaaggcagaatctcaacgaaatctgaacaaagctgggaaaaatggtggtacgaggaacaggaccacctgagaaacaccgggaaggcaggattatttgtggagatcatcttggtcctccggtttttcttcttccagtatgggattgtataccagcttaaaattgcaaacggatccaccagcctttttgtctacttgttctcatggatatacatctttgctatatttgtgctcttcctagtcatccaatacgcccgtgacaagtactcggcaaaagctcacatacggtacaggcttgtccaattcctcctgatcgtgcttgctatactggtgattgttgctttgctcgagttcacgcatttcagcttcatcgatatcttcacaagccttcttgcattcatcccaactggctggggaattctgctgatcgcacagactcaaaggaagtggctgaagaattacactattttctggaatgctgttgtctctgttgctcgcatgtatgacatattgtttgggatactcataatggttccagtagcgttcttgtcatggatgcctggattccagtcaatgcaaacgaggatattattcaatgaagcttttagcagaggacttcgcatcatgcagattgtcactgggaagaaatcaaaaggcgatgtctaagtttaaaaaacggtcttaagaagtaaatggtagttcaaatcctattggtatgtggcgaaggaatcagttggaggtttttggagagtttggttggatgaggaatcgggaagttggtttgattcggttagatgggtttagggagatatttgattgtcagtgtgtgtggagggaactctgattcttgtatggtttttgttctaaaggtacagcaatttgtgtagtgaggctttgtgtatttgttctccttctctcattatagagctttagagcatttttagtttatattcagattgttatct

SEQ ID NO: 35 - protein, 1780 aa, Arabidopsis thaliana
MSLRHRTVPPQTGRPLAAEAVGIEEEPYNIIPVNNLLADHPSLRFPEVRAAAAALKTVGDLRRPPYVQWRSHYDLLDWLALFFGFQKDNVRNQREHMVLHLANAQMRLSPPPDNIDSLDSAVVRRFRRKLLANYSSWCSYLGKKSNIWISDRNPDSRRELLYVGLYLLIWGEAANLRFMPECICYIFHNMASELNKILEDCLDENTGQPYLPSLSGENAFLTGVVKPIYDTIQAEIDESKNGTVAHCKWRNYDDINEYFWTDRCFSKLKWPLDLGSNFFKSRGKSVGKTGFVERRTFFYLYRSFDRLWVMLALFLQAAIIVAWEEKPDTSSVTRQLWNALKARDVQVRLLTVFLTWSGMRLLQAVLDAASQYPLVSRETKRHFFRMLMKVIAAAVWIVAFTVLYTNIWKQKRQDRQWSNAATTKIYQFLYAVGAFLVPEILALALFIIPWMRNFLEETNWKIFFALTWWFQGKSFVGRGLREGLVDNIKYSTFWIFVLATKFTFSYFLQVKPMIKPSKLLWNLKDVDYEWHQFYGDSNRFSVALLWLPVVLIYLMDIQIWYAIYSSIVGAVVGLFDHLGEIRDMGQLRLRFQFFASAIQFNLMPEEQLLNARGFGNKFKDGIHRLKLRYGFGRPFKKLESNQVEANKFALIWNEIILAFREEDIVSDREVELLELPKNSWDVTVIRWPCFLLCNELLLALSQARELIDAPDKWLWHKICKNEYRRCAVVEAYDSIKHLLLSIIKVDTEEHSIITVFFQIINQSIQSEQFTKTFRVDLLPKIYETLQKLVGLVNDEETDSGRVVNVLQSLYEIATRQFFIEKKTTEQLSNEGLTPRDPASKLLFQNAIRLPDASNEDFYRQVRRLHTILTSRDSMHSVPVNLEARRRIAFFSNSLFMNMPHAPQVEKMMAFSVLTPYYSEEVVYSKEQLRNETEDGISTLYYLQTIYADEWKNFKERMHREGIKTDSELWTTKLRDLRLWASYRGQTLARTVRGMMYYYRALKMLAFLDSASEMDIREGAQELGSVRNLQGELGGQSDGFVSENDRSSLSRASSSVSTLYKGHEYGTALMKFTYVVACQIYGSQKAKKEPQAEEILYLMKQNEALRIAYVDEVPAGRGETDYYSVLVKYDHQLEKEVEIFRVKLPGPVKLGEGKPENQNHAMIFTRGDAVQTIDMNQDSYFEEALKMRNLLQEYNHYHGIRKPTILGVREHIFTGSVSSLAWFMSAQETSFVTLGQRVLANPLKVRMHYGHPDVFDRFWFLSRGGISKASRVINISEDIFAGFNCTLRGGNVTHHEYIQVGKGRDVGLNQISMFEAKVASGNGEQVLSRDVYRLGHRLDFFRMLSFFYTTVGFFFNTMMVILTVYAFLWGRVYLALSGVEKSALADSTDTNAALGVILNQQFIIQLGLFTALPMIVEWSLEEGFLLAIWNFIRMQIQLSAVFYTFSMGTRAHYFGRTILHGGAKYRATGRGFVVEHKGFTENYRLYARSHFVKAIELGLILIVYASHSPIAKDSLIYIAMTITSWFLVISWIMAPFVFNPSGFDWLKTVYDFEDFMNWIWYQGRISTKSEQSWEKWWYEEQDHLRNTGKAGLFVEIILVLRFFFFQYGIVYQLKIANGSTSLFVYLFSWIYIFAIFVLFLVIQYARDKYSAKAHIRYRLVQFLLIVLAILVIVALLEFTHFSFIDIFTSLLAFIPTGWGILLIAQTQRKWLKNYTIFWNAVVSVARMYDILFGILIMVPVAFLSWMPGFQSMQTRILFNEAFSRGLRIMQIVTGKKSKGDV

SEQ ID NO: 36 - DNA, 744 bp, Hordeum vulgare
  CDS (1)..(741): Nucleic acid sequence coding for the Bax inhibitor 1 from Hordeum vulgare.
atggacgccttctactcgacctcgtcggcggcggcgagcggctggggccacgactccctcaagaacttccgccagatctcccccgccgtgcagtcccacctcaagctcgtttacctgactctatgctttgcactggcctcatctgccgtgggtgcttacctacacattgccctgaacatcggcgggatgctgacaatgctcgcttgtgtcggaactatcgcctggatgttctcggtgccagtctatgaggagaggaagaggtttgggctgctgatgggtgcagccctcctggaaggggcttcggttggacctctgattgagcttgccatagactttgacccaagcatcctcgtgacagggtttgtcggaaccgccatcgcctttgggtgcttctctggcgccgccatcatcgccaagcgcagggagtacctgtacctcggtggcctgctctcgtctggcctgtcgatcctgctctggctgcagtttgtcacgtccatctttggccactcctctggcagcttcatgtttgaggtttactttggcctgttgatcttcctggggtacatggtgtacgacacgcaggagatcatcgagagggcgcaccatggcgacatggactacatcaagcacgccctcaccctcttcaccgactttgttgccgtcctcgtccgagtcctcatcatcatgctcaagaacgcaggcgacaagtcggaggacaagaagaagaggaagagggggtcctga

SEQ ID NO: 37 - protein, 247 aa, Hordeum vulgare
MDAFYSTSSAAASGWGHDSLKNFRQISPAVQSHLKLVYLTLCFALASSAVGAYLHIALNIGGMLTMLACVGTIAWMFSVPVYEERKRFGLLMGAALLEGASVGPLIELAIDFDPSILVTGFVGTAIAFGCFSGAAIIAKRREYLYLGGLLSSGLSILLWLQFVTSIFGHSSGSFMFEVYFGLLIFLGYMVYDTQEIIERAHHGDMDYIKHALTLFTDFVAVLVRVLIIMLKNAGDKSEDKKKRKRGS

SEQ ID NO: 38 - DNA, 1293 bp, Nicotiana tabacum
  CDS (134)..(880): Nucleic acid sequence coding for the Bax inhibitor 1 from Nicotiana tabacum
ggtggttctatggccttgttcaagttcgaggtttattttgggctcttggtgtttgtgggctatatcatttttgacaccaaggctgttaattgagaaaacatttttggcgtgttgaagaagcaaagagagagaaatggagtcttgcacatcgttcttcaattcacagtcggcgtcgtctcgcaatcgctggagttacgattctcttaagaacttccgccagatctctccctttgttcaaactcatctcaaaaaggtctacctttcattatgttgtgctttagttgcttcggctgctggagcttaccttcacattctttggaacattggtggcttacttacgacattgggatgtgtgggaagcatagtgtggctgatggcgacacctctgtatgaagagcaaaagaggatagcacttctgatggcagctgcactgtttaaaggagcatctattggtccactgattgaattggctattgactttgacccaagcattgtgatcggtgcttttgttggttgtgctgtggcttttggttgcttctcagctgctgccatggtggcaaggcgcagagagtacttgtatcttggaggtcttctttcatctggtctctctatccttttctggttgcacttcgcgtcctccatttttggtggttctatggccttgttcaagttcgaggtttattttgggctcttggtgtttgtgggctatatcatttttgacacccaagatataattgagaaggcacaccttggggatttggactacgtgaagcatgctctgaccctctttacagattttgttgctgtttttgtgcgaatattaatcataatgctgaagaatgcatccgacaaggaagagaagaagaagaagaggagaaactaatgcataagcggttattcaaagactctgtaactctagaatctggcattttcttgttcataaacttctgtagaccttcgacaagtatgttgttaatagtttggtaacgcctcagattaagctgcgaggctctgttatgccgcatgccaatgtggttatggtggtacatagatggttttgtttccgaagcataccatcaaataacatgcatgtttacactatatcgataacctacgagtgtactacttatttctgctcccttttgctgtgttaggttgttcatgattgtatagttgattttccgttatgttagaccatcttctttcttgacgtttaatttctcatattgatgggagaaatgaaaattcacaccgtcgccccaacttgtttaagactgaggcgcaattgtagtt

SEQ ID NO: 39 - protein, 249 aa, Nicotiana tabacum
MESCTSFFNSQSASSRNRWSYDSLKNFRQISPFVQTHLKKVYLSLCCALVASAAGAYLHILWNIGGLLTTLGCVGSIVWLMATPLYEEQKRIALLMAAALFKGASIGPLIELAIDFDPSIVIGAFVGCAVAFGCFSAAAMVARRREYLYLGGLLSSGLSILFWLHFASSIFGGSMALFKFEVYFGLLVFVGYIIFDTQDIIEKAHLGDLDYVKHALTLFTDFVAVFVRILIIMLKNASDKEEKKKKRRN

SEQ ID NO: 40 - DNA, 23 bp, artificial sequence
  misc_feature: Oligonucleotide primer Heil 131
gttcgccgtttcctcccgcaact

SEQ ID NO: 41 - DNA, 26 bp, artificial sequence
  misc_feature: Oligonucleotide primer Gene Racer 5′-nested primer,invitrogen
ggacactgacatggactgaaggagta

SEQ ID NO: 42 - DNA, 27 bp, artificial sequence
  misc_feature: Oligonucleotide primer RACE-HvCSL1
gcccaacatctcttcctttaccaacct

SEQ ID NO: 43 - DNA, 23 bp, artificial sequence
  misc_feature: Oligonucleotide primer GeneRacer 5′-Primer
cgactggagcacgaggacactga

SEQ ID NO: 44 - DNA, 27 bp, artificial sequence
  misc_feature: Oligonucleotide primer RACE-5′nested HvCSL1
tctggctttatctggtgttggagaatc

SEQ ID NO: 45 - DNA, 25 bp, artificial sequence
  misc_feature: Oligonucleotide primer GeneRacer 3′-Primer
gctgtcaacgatacgctacgtaacg

SEQ ID NO: 46 - DNA, 23 bp, artificial sequence
  misc_feature: Oligonucleotide primer GeneRacer 3′-Nested Primer
cgctacgtaacggcatgacagtg

SEQ ID NO: 47 - DNA, 18 bp, artificial sequence
  misc_feature: Oligonucleotide primer M13-fwd
gtaaaacgacggccagtg

SEQ ID NO: 48 - DNA, 19 bp, artificial sequence
  misc_feature: Oligonucleotide primer M13-Rev
ggaaacagctatgaccatg

SEQ ID NO: 49 - DNA, 23 bp, artificial sequence
  misc_feature: Oligonucleotide primer Hei 97 forward
ttgggcttaatcagatcgcacta

SEQ ID NO: 50 - DNA, 23 bp, artificial sequence
  misc_feature: Oligonucleotide primer Hei 98 reverse
gtcaaaaagttgcccaagtctgt

(19) United States
(12) Patent Application Publication
(10) Pub. No.: US 2008/0120740 A1
(43) Pub. Date: May 22, 2008

(54) Novel Nucleic Acid Sequences and Their Use in Methods for Achieving a Pathogenic Resistance in Plants

(75) Inventors: Markus Frank, Mannheim (DE); Ralf-Michael Schmidt, Kirrweiler (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 11/596,448

(22) PCT Filed: May 6, 2005
(86) PCT No.: PCT/EP05/04916

§ 371 Date: Nov. 13, 2006

(30) Foreign Application Priority Data
May 13, 2004 (DE) 10 2004 024 184.8

(51) Int. Cl. C12N 15/87; A01H 1/00; C12N 15/00; C07H 21/04; C12N 5/02; A01H 5/00

(52) U.S. Cl. 800/279; 800/278; 530/350; 536/22.1; 536/23.1; 435/320.1; 435/419; 800/298; 800/320; 800/320.1; 800/320.2; 800/320.3; 435/468

(57) ABSTRACT

A process for increasing the resistance against mesophyllic cell-penetrating pathogens in a plant, or an organ, tissue or a cell thereof, wherein the callose synthase activity in the plant or an organ, tissue or a cell thereof is reduced in comparison to control plants.

The invention relates inter alia to novel polypeptides and nucleic acid sequences coding therefor from plants and expression cassettes and vectors which comprise these sequences. The invention further relates to transgenic plants transformed with these expression cassettes or vectors, and cultures, parts or transgenic reproductive material derived therefrom. The invention further relates to processes for the creation or increasing of pathogen resistance in plants by reduction of the expression of at least one callose synthase polypeptide or of a functional equivalent thereof.

The aim of biotechnology work in plants is the creation of plants with advantageous, novel properties, for example for increasing agricultural productivity, for quality improvement in foodstuffs or for the production of certain chemicals or pharmaceuticals (Dunwell J M (2000) J Exp Bot 51 Spec No:487-96). The natural defense mechanisms of plants against pathogens are often insufficient. Fungal diseases alone result in crop losses to the extent of many billions of US $ per year. The introduction of foreign genes from plants, animals or microbial sources can strengthen the defense. Examples are protection against insect damage in tobacco through expression of Bacillus thuringiensis endotoxins under control of the 35 S CaMV promoter (Vaeck et al. (1987) Nature 328:33-37) or protection of tobacco against fungal attack through expression of a chitinase from the bean under control of the CaMV promoter (Broglie et al. (1991) Science 254:1194-1197). However, most of the approaches described only ensure resistance against a single pathogen or against a narrow spectrum of pathogens.

There are only a few approaches which impart resistance to pathogens, in particular fungal pathogens, to plants. The reason for this is the complexity of the biological systems in question. An obstacle to the attainment of resistances to pathogens is the fact that the interactions between pathogen and plant are very complex and extremely species- or genus-specific. The large number of different pathogens, the different infection mechanisms developed by these organisms and the specific defense mechanisms developed by the plant strains, families and species can be regarded as significant factors in this.

Fungal pathogens have essentially developed two quite different infection strategies. Many fungi penetrate into the host tissue via the stomata (e.g. rust fungi, Septoria and Fusarium species) and penetrate the mesophyllic tissue, while others penetrate via the cuticles of the epidermal cells lying thereunder (e.g. Blumeria species).

In plants, infections result in the development of various defense mechanisms. These mechanisms can be very diverse, depending on the plant/pathogen system in question.

Thus it could be shown that defense reactions against epidermis-penetrating fungi often begin with the development of a penetration resistance (formation of papillae, cell wall thickening with callose as the main component) beneath the fungal penetration hypha (Elliott et al. Mol Plant Microbe Interact. 15: 1069-77; 2002). Various approaches have hitherto been described for the creation/increasing of resistance to epidermis-penetrating fungal pathogens.

Thus, enhanced resistance to many species of mildew is said to be attained by inhibition of the expression of the mlo gene (Büschges R et al. (1997) Cell 88:695-705; Jorgensen J H (1977) Euphytica 26:55-62; Lyngkjaer M F et al. (1995) Plant Pathol 44:786-790). The Mlo-mediated resistance is said to result from the formation of papillae (cell wall thickening with callose as the main component) beneath the penetration site of the pathogen, the epidermal cell wall. A disadvantage in Mio-mediated resistance is the fact that, even in the absence of a pathogen, Mlo-deficient plants initiate a defense mechanism which for example manifests itself in spontaneous necrosis of leaf cells (Wolter M et al. (1993) Mol Gen Genet 239:122-128), which may explain the increased susceptibility to necrotrophic or hemibiotrophic pathogens.

A heightening of pathogen defense in plants against necrotrophic or hemibiotrophic fungal pathogens should be attainable by increasing the activity of a Bax inhibitor-1 protein in the mesophyllic tissue of plants.

This development of penetration resistance against pathogens whose mechanism of infection includes penetration of the epidermal cells is possibly of especial importance for monocotyledonous plants in particular. The analysis of A. thaliana plants in which the expression of GSL-5 (codes for a callose synthase) had been suppressed by a loss of function mutation (Nishimura et al. Science, 2003 Aug. 15; 301 (5635):969-72) or by induction of post-transcriptional gene silencing (PTGS) (Jacobs et al. Plant Cell. 2003 November; 15(11):2503-13) has shown that these plants display greatly reduced papillar callose formation and increased resistance to epidermis-penetrating virulent mildew species, e.g. Erysiphe cichoracearum. At the same time, these plants exhibit a slightly increased susceptibility to the powdery mildew species Blumeria graminis, which is also epidermis-penetrating.

Thus, in monocotyledonous and dicotyledonous plants, as well as common features, there are also fundamental differences in the defense reactions induced by pathogen attack.

The penetration barrier well-known from the defense reaction against epidermis-penetrating pathogens appears to have no significance in the case of mesophyllic tissue-penetrating pathogens (e.g. rusts, Septoria or Fusarium species) (e.g. Scharen, in Septoria and Stagonospora diseases of wheat, eds. Van Ginkel, McNab, pp. 19-22).

At present, there is no known method with which resistance of plants can be created towards pathogens that infect plants by penetration into plant guard cells with subsequent penetration of the mesophyllic tissue.

Hence the problem on which the present invention was based was to provide a method for the creation of resistance of plants to mesophyllic cell-penetrating pathogens.

The solution of the problem is solved by the embodiments characterized in the claims.

Accordingly, the invention relates to a process for increasing the resistance against mesophyllic cell-penetrating pathogens in a plant, or an organ, tissue or a cell thereof, wherein the callose synthase activity in the plant or an organ, tissue or a cell thereof is reduced in comparison to control plants. In a particular embodiment, the pathogens are selected from the Pucciniaceae, Mycosphaerellaceae and Hypocreaceae families.

It is surprising that the cDNA sequences coding for callose synthases disclosed here according to the invention, has the consequence, e.g. after gene silencing via dsRNAi, of an increase in the resistance against fungal pathogens which penetrate into plants via stomata and then penetrate the mesophyllic tissue, in particular against pathogens from the Pucciniaceae, Mycosphaerellaceae and Hypocreaceae families. Preferably the plant is a monocotyledonous plant.

For the callose synthases from barley (Hordeum vulgare), wheat (Triticum aestivum) and maize (Zea mays), a negative control function is presumed in case of attack by mesophyllic cell-penetrating pathogens. The reduction of expression of a callose synthase in the cell by a sequence-specific RNA interference approach with the use of double-stranded callose synthase dsRNA (“gene silencing”) can diminish the infection of the mesophyllic tissue with phytopathogenic fungi, in particular of the Pucciniaceae, Mycosphaerellaceae and Hypocreaceae families.

In one embodiment, the reduction of the activity of the callose synthase polypeptide is effected specifically to mesophyllic tissue, for example by recombinant expression of a nucleic acid molecule coding for said callose synthase polypeptide for the induction of a co-suppression effect under control of a mesophyllic tissue-specific promoter.

In a further embodiment, the decrease in the quantity of polypeptide, activity or function of a callose synthase in a plant is effected in combination with an increase in the quantity of polypeptide, activity or function of a Bax inhibitor-1 protein (BI-1), preferably of the Bax inhibitor-1 protein from Hordeum vulgare (GenBank Acc.-No.: AJ290421, SEQ ID No: 37) or the Bax inhibitor-1 protein from Nicotiana tabacum (GenBank Acc.-No.: AF390556, SEQ ID No: 39). This can for example be effected by expression of a nucleic acid molecule coding for a Bax inhibitor-1 polypeptide, e.g. in combination with a tissue-specific increase in the activity of a Bax inhibitor-1 protein in the mesophyllic tissue. The reduction in the callose synthase activity in a transgenic plant which over-expresses BI-1 in the mesophyllic tissue has the consequence that both biotrophic and also necrotrophic fungi can successfully be defended against. Thus this combination offers the opportunity of generating comprehensive fungal resistance in the plant. Nucleic acid molecules which are suitable for expression of the BI-1 are for example BI1 genes from rice (GenBank Acc.-No.: AB025926), Arabidopsis (GenBank Acc.-No.: AB025927), tobacco and rape (GenBank Acc.-No.: AF390555, Bolduc N et al. (2003) Planta 216:377-386).

Since callose polymers are an important metabolic product of higher plants and are synthesized in the course of the formation of pollen tubes, phragmoplasts, papillae or as a sealing material for cell wall pores, and in the cribriform plates of the phloem components, an ubiquitous distribution of callose synthase polypeptides in plants is to be presumed. For this reason, the process according to the invention can in principle be applied to all plant species.

The sequences from other plants homologous to the callose synthase sequences disclosed in the context of this invention can easily be found for example by database searches or by scrutiny of gene banks using the callose synthase sequences as search sequence or probe.

“Plants” in the context of the invention means all dicotyledonous or monokyledonic plants. Preferred are plants which can be subsumed under the class of the Liliatae (Monocotyledoneae or monocotyledonous plants). Included under the term are the mature plants, seeds, shoots and embryos, and parts, reproductive material, plant organs, tissues, protoplasts, calluses and other cultures, for example cell cultures, derived therefrom, and all other types of groupings of plant cells into functional or structural units. [Mature plants means plants at any development stage beyond the embryo. Embryo means a young, immature plant at an early development stage].

“Plant” also comprises annual and perennial dicotyledonous or monocotyledonous plants and includes, by way of example, but not restrictively, those of the genera Bromus, Asparagus, Pennisetum, Lolium, Oryza, Zea, Avena, Hordeum, Secale, Triticum, Sorghum and Saccharum.

In a preferred embodiment, the process is applied to monocotyledonous plants, for example from the Poaceae family, particularly preferably to the genera Oryza, Zea, Avena, Hordeum, Secale, Triticum, Sorghum, and Saccharum, very particularly preferably to plants of agricultural significance, such as for example Hordeum vulgare (barley), Triticum aestivum (wheat), Triticum aestivum subsp. spelta (spelt), Triticale, Avena sative (oats), Secale cereale (rye), Sorghum bicolor (millet), Zea mays (maize), Saccharum officinarum (sugar cane) or Oryza sative (rice).

“Mesophyllic tissue” means the leaf tissue lying between the epidermal layers, consisting of the palisade tissue, the spongy parenchyma and the leaf veins.

“Nucleic acids” means biopolymers of nucleotides which are linked together via phosphodiester bonds (polynucleotides, polynucleic acids). Depending on the type of sugar in the nucleotides (ribose or desoxyribose), the distinction is made between the two classes, the ribonucleic acids (RNA) and the desoxyribonucleic acids (DNA).

The term “crop” means all plant parts obtained by agricultural cultivation of plants and collected in the course of the harvesting procedure.

“Resistance means the reduction or weakening of disease symptoms of a plant due to an attack by a pathogen. The symptoms can be of a diverse nature, but preferably comprise those which directly or indirectly result in impairment of the quality of the plant, the quantity of the yield, the suitability for use as a fodder or foodstuff, or else hinder the sowing, cultivation, harvesting or processing of the harvested product.

“Imparting”, “existence”, “generation” or “increasing” of a pathogen resistance means that through the use of the process according to the invention the defense mechanisms of a certain plant species or variety displays an increased resistance against one and more pathogens, compared to the wild type of the plant (“control plant” or “starting plant”), on which the process according to the invention was not used, under otherwise identical conditions (such as for example climatic or cultivation conditions, pathogen species, etc.). Here, the increased resistance preferably manifests itself as decreased development of the disease symptoms, where disease symptoms, as well as the adverse effects mentioned above, also for example comprises the penetration efficiency of a pathogen into the plant or plant cell or the proliferation efficiency in or on the same. Here the disease symptoms are preferably decreased by at least 10% or at least 20%, particularly preferably by at least 40% or 60%, very particularly preferably by at least 70% or 80%, most preferably by at least 90% or 95%.

In the context of the invention, “pathogen” means organisms the interactions whereof with a plant result in the disease symptoms described above, and in particular means pathogenic organisms from the fungal kingdom. Preferably, pathogen is understood to mean a mesophyllic tissue-penetrating pathogen, particularly preferably pathogens which penetrate into plants via the stomata and then penetrate the mesophyllic tissue. Preferably mentioned here are organisms of the strains Ascomycota and Basidomycota. Particularly preferable here are the Pucciniaceae, Mycosphaerellaceae and Hypocreaceae families.

Particularly preferred are organisms of these families which belong to the genera Puccinia, Fusarium or Mycosphaerella.

Very particularly preferred are the species Puccinia triticina, Puccinia striiformis, Mycosphaerella graminicola, Stagonospora nodorum, Fusarium graminearum, Fusarium culmorum, Fusarium avenaceum, Fusarium poae and Microdochium nivale.

It can however be presumed that the reduction of the expression of a callose synthase polypeptide, its activity or function also results in resistance to other pathogens. Changes in the cell wall structure may represent a fundamental mechanism of pathogen resistance.

Particularly preferred are Ascomycota such as for example Fusarium oxysporum (Fusarium wilt in tomatoes), Septoria nodorum and Septoria tritici (blotch in wheat), Basidiomycetes such as for example Puccinia graminis (black rust in wheat, barley, rye and oats), Puccinia recondita (brown rust in wheat), Puccinia dispersa (brown rust in rye), Puccinia hordei (brown rust in barley) and Puccinia coronata (crown rust in oats).

In one embodiment, the process according to the invention results in resistance in

barley against the pathogen:

### Puccinia graminis f.sp. hordei (barley stem rust),

in wheat against the pathogens:
Fusarium graminearum, Fusarium avenaceum, Fusarium culmorum, Puccinia graminis f.sp. tritici, Puccinia recondita f.sp. tritici, Puccinia striiformis, Septoria nodorum, Septoria tritici, Septoria avenae or Puccinia graminis f.sp. tritici (wheat stem rust),
in maize against the pathogens:

### Fusarium moniliforme var. subglutinans, Puccinia sorghi or Puccinia polysora,

and in sorghum against the pathogens:

### Puccinia purpurea, Fusarium moniliforme, Fusarium graminearum or Fusarium oxysporum.

In the context of the invention, “callose synthase polypeptide” means a protein with the activity described below. In one embodiment the invention relates to a callose synthase polypeptide, e.g. a callose synthase polypeptide from barley according to SEQ ID No: 2, 4, 6 or 8 and/or its homolog from maize (Zea mays) SEQ ID No: 10, 11, 13, 15 or 17 and/or from rice (Oryza sative) according to SEQ ID No: 19 or 21 and/or wheat (Triticum aestivum) according to SEQ ID No: 23, 25, 27, 29, 31 and/or 33 and/or A. thaliana SEQ ID No: 34 or a fragment thereof. In one embodiment the invention relates to functional equivalents of the aforesaid polypeptide sequences.

“Quantity of polypeptide” means for example the quantity of calloses synthase polypeptides in an organism, a tissue, a cell or a cell compartment. “Reduction” in the quantity of polypeptide means the quantitative reduction in the quantity of callose synthase polypeptides in an organism, a tissue, a cell or a cell compartment—for example by one of the processes described below—compared to the wild type (control plant) of the same genus and species on which this process was not used, under otherwise identical boundary conditions (such as for example cultivation conditions, age of the plants etc.). The reduction here is at least 10%, preferably at least 10% or at least 20%, particularly preferably at least 40% or 60%, very particularly preferably at least 70% or 80%, most preferably at least 90% or 99%.

“Activity” or “function” of a callose synthase polypeptide means the formation or synthesis of linear β-1→3 glycosidically linked glucan polymers, which can also display 1→6 glycosidically or 1→4 glycosidically linked branchings (callose polymers).

“Reduction” of the activity or function of a callose synthase means for example the reduction of the ability to synthesize or lengthen callose polymers in a cell, a tissue or an organ, for example by one of the processes described below, in comparison to the wild type of the same genus and species on which this process was not used, under otherwise identical boundary conditions (such as for example cultivation conditions, age of the plants etc.). The reduction here is at least 10%, preferably at least 10% or at least 20%, particularly preferably by at least 40% or 60%, very particularly preferably by at least 70% or 80%, most preferably by at least 90%, 95% or more. Reduction should be understood also to mean the alteration of the substrate specificity, such as can for example be expressed by the kcat/Km value. The reduction here is at least 10%, preferably at least 10% or at least 20%, particularly preferably by at least 40% or 60%, very particularly preferably by at least 70% or 80%, most preferably by at least 90%, 95% or more.

Methods for the detection of callose polymers formed as a result of biotic or abiotic stress are well known to the skilled person, and have been described many times (inter alia: Jacobs et al., The Plant Cell, Vol. 15, 2503-13, 2003; Desprez et al., Plant Physiology, 02. 2002, Vol. 128, pp. 482-490). Callose deposits can be made visible in tissue sections for example by staining with aniline blue. Callose stained with aniline blue is recognizable by the yellow fluorescence of the aniline blue fluorochrome, induced by UV light.

A further object of the present invention is the generation of pathogen resistance by reduction of the function, activity or quantity of polypeptide of at least one callose synthase polypeptide comprising the sequences shown in SEQ ID No: 2, 4, 6, 8, 10, 11, 13, 15, 17, 19, 21, 23, 25, 27, 29, 31, 33 and/or 35 and/or of a polypeptide which displays a homology thereto of at least 40% and/or of a functional equivalent of the aforesaid polypeptide.

Homology between two nucleic acid sequences is understood to mean the identity of the nucleic acid sequence over the whole sequence length in question, which is calculated by comparison with the aid of the program algorithm GAP (Wisconsin Package Version 10.0, University of Wisconsin, Genetics Computer Group (GCG), Madison, USA; Altschul et al. (1997) Nucleic Acids Res. 25:3389ff) with insertion of the following parameters:

| | |
|---|---|
| Gap Weight: 50 | Length Weight: 3 |
| Average Match: 10 | Average Mismatch: 0 |

By way of example, a sequence which displays a homology of at least 80% on a nucleic acid basis with the sequence SEQ ID No: 1 is understood to mean a sequence which on comparison with the sequence SEQ ID No: 1 in accordance with the above program algorithm with the above parameter set displays a homology of at least 80%.

Homology between two polypeptides is understood to mean the identity of the amino acid sequence over the whole sequence length in question, which is calculated by comparison with the aid of the program algorithm GAP (Wisconsin Package Version 10.0, University of Wisconsin, Genetics Computer Group (GCG), Madison, USA) with insertion of the following parameters:

| | |
|---|---|
| Gap Weight: 8 | Length Weight: 2 |
| Average Match: 2,912 | Average Mismatch: −2,003 |

By way of example, a sequence which displays a homology of at least 80% on a polypeptide basis with the sequence SEQ ID No: 2 is understood to mean a sequence which on comparison with the sequence SEQ ID No: 2 in accordance with the above program algorithm with the above parameter set displays a homology of at least 80%.

In a preferred embodiment of the present invention, the callose synthase activity available to the plant, the plant organ, tissue or the cell is reduced in that the activity, function or quantity of polypeptide of at least one polypeptide in the plant, the plant organ, tissue or the cell is reduced, which is encoded by a nucleic acid molecule comprising a nucleic acid molecule selected from the group consisting of:
a) a nucleic acid molecule which encodes a polypeptide comprising the sequence shown in SEQ ID No: 2, 4, 6, 8, 10, 11, 13, 15, 17, 19, 21, 23, 25, 27, 29, 31, 33 and/or 35;
b) a nucleic acid molecule which comprises at least one polynucleotide of the sequence according to SEQ ID No: 1, 3, 5, 7, 9, 12, 14, 16, 18, 20, 22, 24, 26, 28, 30, 32 and/or 34;
c) a nucleic acid molecule which encodes a polypeptide the sequence whereof displays an identity of at least 40% with the sequences SEQ ID No: 2, 4, 6, 8, 10, 11, 13, 15, 17, 19, 21, 23, 25, 27, 29, 31, 33 and/or 35;
d) a nucleic acid molecule according to (a) to (c) which codes for a fragment or an epitope of the sequences according to SEQ. ID No.: 2, 4, 6, 8, 10, 11, 13, 15, 17, 19, 21, 23, 25, 27, 29, 31, 33 and/or 35;
e) a nucleic acid molecule which encodes a polypeptide which is recognized by a monoclonal antibody directed against a polypeptide which is encoded by the nucleic acid molecules according to (a) to (c); and
f) a nucleic acid molecule coding for a callose synthase, which hybridizes under stringent conditions with a nucleic acid molecule according to (a) to (c) or part fragments thereof consisting of at least 15 nucleotides (nt), preferably 20 nt, 30 nt, 50 nt, 100 nt, 200 nt or 500 nt;
g) a nucleic acid molecule coding for a callose synthase, which can be isolated from a DNA bank with the use of a nucleic acid molecule according to (a) to (c) or part fragments thereof of at least 15 nt, preferably 20 nt, 30 nt, 50 nt, 100 nt, 200 nt or 500 nt as a probe under stringent hybridization conditions;
   comprises a complementary sequence thereof, or represents a functional equivalent.

Preferably, the activity of said polypeptides in the mesophyllic cells of a plant is reduced as explained above.

“Epitope” is understood to mean the regions of an antigen determining the specificity of the antibody (the antigenic determinant).

An epitope is therefore the part of an antigen which actually comes into contact with the antibody.

Such antigenic determinants are the regions of an antigen to which the T-cell receptors react and as a result produce antibodies which specifically bind the antigen determinant/epitope of an antigen. Antigens or their epitopes are therefore capable of inducing the immune response of an organism resulting in the formation of specific antibodies directed against the epitope. Epitopes for example consist of linear sequences of amino acids in the primary structure of proteins, or of complex secondary or tertiary protein structures. A hapten is understood to mean an epitope detached from the context of the antigenic environment. Although by definition haptens have an antibody directed against them, under some circumstances haptens are not capable of inducing an immune response after for example injection into an organism. For this purpose, haptens are coupled to carrier molecules. As an example, dinitrophenol (DNP) may be mentioned, which was used for the preparation of antibodies directed against DNP after coupling to BSA (bovine serum albumin) (Bohn, A., König, W. 1982)

Haptens are therefore (often small molecule) substances which trigger no immune reaction themselves, but do so very well when they have been coupled to large molecule carriers. The antibodies thus created also include ones that can bind the hapten by themselves.

Antibodies in the context of the present invention can be used for the identification and isolation of polypeptides disclosed according to the invention from organisms, preferably plants, particularly preferably monocotyledonous plants. The antibodies can be both of a monoclonal, polyclonal, or synthetic nature, or consist of antibody fragments such as Fab, Fv or scFv fragments, which are formed by proteolytic degradation. “Single chain” Fv (scFv) fragments are single-chain fragments, which comprise only the variable regions of the heavy and light antibody chains, linked via a flexible linker sequence. Such scFv fragments can also be produced as recombinant antibody derivatives. Presentation of such antibody fragments on the surface of filamentous phages enables the direct selection of high-affinity binding scFv molecules from combinatorial phage libraries.

Monoclonal antibodies can be obtained in accordance with the method described by Köhler and Milstein (Nature 256 (1975), 495).

“Functional equivalents” of a callose synthase polypeptide preferably means those polypeptides which display a homology of at least 40% to the polypeptides described by the sequences SEQ. ID No.: 2, 4, 6, 8, 10, 11, 13, 15, 17, 19, 21, 23, 25, 27, 29, 31, 33 and/or 35 and essentially display the same properties or have the same function.

“Essentially the same properties” of a functional equivalent means above all the imparting of a pathogen-resistant phenotype or the imparting or heightening of the pathogen resistance against at least one pathogen with reduction of the quantity of polypeptide, activity or function of said functional callose synthase equivalent in a plant, organ, tissue, part or cells, in particular in mesophyllic cells thereof.

Here the efficiency of the pathogen resistance can deviate both downwards and also upwards compared to a value obtained with reduction of one of the callose synthase polypeptides according to SEQ. ID No.: 2, 4, 6, 8, 10, 11, 13, 15, 17, 19, 21, 23, 25, 27, 29, 31, 33 and/or 35. Those functional equivalents with which the efficiency of the pathogen resistance, measured for example by the penetration efficiency of a pathogen, does not deviate by more than 50%, preferably 25%, particularly preferably 10% from a comparison value which is obtained by reduction of a callose synthase polypeptide according SEQ. ID No.: 2, 4, 6, 8, 10, 11, 13, 15, 17, 19, 21, 23, 25, 27, 29, 31, 33 and/or 35 are preferred. Those sequences with the reduction whereof the efficiency of the pathogen resistance quantitatively exceeds by more than 50%, preferably 100%, particularly preferably 500%, very particularly preferably 1000% a comparison value obtained by reduction of one of callose synthase polypeptides according to SEQ. ID No.: 2, 4, 6, 8, 10, 11, 13, 15, 17, 19, 21, 23, 25, 27, 29, 31, 33 and/or 35 are particularly preferred.

The comparison is preferably performed under analogous conditions.

“Analogous conditions” means that all boundary conditions such as for example cultivation or growing conditions, assay conditions (such as buffer, temperature, substrates, pathogen concentration etc.) are maintained identical between the tests to be compared and the preparations differ only in the sequence of the callose synthase polypeptides to be compared, their source organism and if appropriate the pathogen.

“Functional equivalents” also means natural or artificial mutation variants of the callose synthase polypeptides according to SEQ. ID No.: 2, 4, 6, 8, 10, 11, 13, 15, 17, 19, 21, 23, 25, 27, 29, 31, 33 and/or 35 and homologous polypeptides from other monocotyledonous plants which still display essentially the same properties. Homologous polypeptides from preferred plants described above are preferred. The sequences from other plants (for example Oryza sative) homologous to the callose synthase sequences disclosed in the context of this invention can easily be found for example by database searches or by scrutiny of gene banks using the callose synthase sequences as search sequence or probe.

Functional equivalents can for example also be derived from one of the polypeptides according to the invention according to SEQ. ID No.: 2, 4, 6, 8, 10, 11, 13, 15, 17, 19, 21, 23, 25, 27, 29, 31, 33 and/or 35 by substitution, insertion or deletion, and display a homology to these polypeptides of at least 60%, preferably at least 80%, preferably at least 90%, particularly preferably at least 95%, very particularly preferably at least 98% and are characterized by essentially the same properties as the polypeptides according to SEQ. ID No.: 2, 4, 6, 8, 10, 11, 13, 15, 17, 19, 21, 23, 25, 27, 29, 31, 33 and/or 35.

Functional equivalents are also nucleic acid molecules derived from the nucleic acid sequences according to the invention according to SEQ ID No: 1, 3, 5, 7, 9, 12, 14, 16, 18, 20, 22, 24, 26, 28, 30, 32 and/or 34 by substitution, insertion or deletion, and have a homology of at least 60%, preferably 80%, preferably at least 90%, particularly preferably at least 95%, very particularly preferably at least 98% to one of the polynucleotides according to the invention according to SEQ. ID No: 1, 3, 5, 7, 9, 12, 14, 16, 18, 20, 22, 24, 26, 28, 30, 32 and/or 34 and code for polypeptides with essentially identical properties to polypeptides according to SEQ. ID No.: 2, 4, 6, 8, 10, 11, 13, 15, 17, 19, 21, 23, 25, 27, 29, 31, 33 or 35.

Examples of the functional equivalents of the callose synthases according to SEQ. ID No.: 2, 4, 6, 8, 10, 11, 13, 15, 17, 19, 21, 23, 25, 27, 29, 31, 33 and/or 35 to be reduced in the process according to the invention can for example be found from organisms whose genomic sequence is known, for example from Oryza sative by homology comparisons from databases.

The scrutiny of cDNA or genomic libraries of other organisms, preferably of the plant species mentioned below as suitable as hosts for the transformation, with the use of the nucleic acid sequence described under SEQ. ID No: 1, 3, 5, 7, 9, 12, 14, 16, 18, 20, 22, 24, 26, 28, 30, 32 and/or 34 or parts thereof as probe, is also a process familiar to the skilled person, for identifying homologs in other species. Here the probes derived from the nucleic acid sequence according to SEQ. ID No: 1, 3, 5, 7, 9, 12, 14, 16, 18, 20, 22, 24, 26, 28, 30, 32 and/or 34 have a length of at least 20 bp, preferably at least 50 bp, particularly preferably at least 100 bp, very particularly preferably at least 200 bp, most preferably at least 400 bp. The probe can also be one or several kilobases long, e.g. 1 Kb, 1.5 Kb or 3 Kb. For the scrutiny of the libraries, a DNA strand complementary to the sequences described under SEQ. ID No: 1, 3, 5, 7, 9, 12, 14, 16, 18, 20, 22, 24, 26, 28, 30, 32 and/or 34, or a fragment thereof with a length between 20 Bp and several kilobases can also be used.

In the process according to the invention, DNA molecules can also be used which under standard conditions hybridize with the nucleic acid molecules described by SEQ. ID No: 1, 3, 5, 7, 9, 12, 14, 16, 18, 20, 22, 24, 26, 28, 30, 32 and/or 34 and coding for callose synthases, the nucleic acid molecules complementary to these or parts of the aforesaid and code as complete sequences for polypeptides which possess the same properties as the polypeptides described under SEQ. ID No.: 2, 4, 6, 8, 10, 11, 13, 15, 17, 19, 21, 23, 25, 27, 29, 31, 33 and/or 35.

“Standard hybridization conditions” should be broadly understood and depending on the use means stringent and also less stringent hybridization conditions. Such hybridization conditions are inter alia described in Sambrook J, Fritsch E F, Maniatis T et al., in Molecular Cloning (A Laboratory Manual), 2^{nd} Edn., Cold Spring Harbor Laboratory Press, 1989, pages 9.31-9.57) or in Current Protocols in Molecular Biology, John Wiley & Sons, N.Y. (1989), 6.3.1-6.3.6.

The skilled person would select hybridization conditions which enable him to distinguish specific from nonspecific hybridizations.

For example, the conditions during the washing step can be selected from conditions with low stringency (with about 2×SSC at 50° C.) and those with higher stringency (with about 0.2×SSC at 50° C. preferably at 65° C.) (20×SSC: 0.3M sodium citrate, 3M NaCl, pH 7.0). Furthermore, the temperature during the washing step can be raised from low stringency conditions at room temperature, about 22° C., to more severe stringency conditions at about 65° C. The two parameters, salt concentration and temperature, can be varied simultaneously or also individually, in which case the respective other parameter is maintained constant. During the hybridization, denaturing agents such as for example formamide or SDS can also be used. In the presence of 50% formamide, the hybridization is preferably performed at 42° C. Some examples of conditions for hybridization and washing step are given below:
1. Hybridization conditions can for example be selected from the following conditions:
   a) 4×SSC at 65° C.,
   b) 6×SSC at 45° C.,
   c) 6×SSC, 100 μg/ml of denatured, fragmented fish sperm DNA at 68° C.,
   d) 6×SSC, 0.5% SDS, 100 μg/ml of denatured salmon sperm DNA at 68° C.,
   e) 6×SSC, 0.5% SDS, 100 μg/ml of denatured, fragmented salmon sperm DNA, 50% formamide at 42° C.
   f) 50% formamide, 4×SSC at 42° C., or
   g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
   h) 2× or 4×SSC at 50° C. (low stringency condition),
      30 to 40% formamide, 2× or 4×SSC at 42° C. (low stringency condition).
      500 mN sodium phosphate buffer pH 7.2, 7% SDS (g/V), 1 mM EDTA, 10 μg/ml single stranded DNA, 0.5% BSA (g/V) (Church and Gilbert, Genomic sequencing. Proc. Natl. Acad. Sci. U.S.A. 81:1991. 1984)
2. Washing steps can for example be selected from the following conditions:
   a) 0.015 M NaCl/0.0015 M sodium citrate/0.1% SDS at 50° C.
   b) 0.1×SSC at 65° C.
   c) 0.1×SSC, 0.5% SDS at 68° C.
   d) 0.1×SSC, 0.5% SDS, 50% formamide at 42° C.
   e) 0.2×SSC, 0.1% SDS at 42° C.
   f) 2×SSC at 65° C. (low stringency condition).

In one embodiment, the hybridization conditions are selected as follows:

A hybridization buffer is selected which comprises formamide, NaCl and PEG 6000. The presence of formamide in the hybridization buffer destabilizes double strand nucleic acid molecules, as a result of which the hybridization temperature can be lowered to 42° C., without thereby lowering the stringency. The use of salt in the hybridization buffer increases the renaturation ratio of a duplex, or the hybridization efficiency. Although PEG increases the viscosity of the solution, which has an adverse effect on renaturation ratios, the concentration of the probe in the remaining medium is increased by the presence of the polymer in the solution, which increases the hybridization ratio. The composition of the buffer is as follows:

Hybridization buffer

| | |
|---|---|
| 250 | mM sodium phosphate buffer pH 7.2 |
| 1 | mM EDTA |
| 7% | SDS (w/v) |
| 250 | mM NaCl |
| 10 | μg/ml ssDNA |
| 5% | polyethylene glycol (PEG) 6000 |
| 40% | formamide |

The hybridizations are performed at 42° C. overnight. On the following morning, the filters are washed 3× with 2×SSC+0.1% SDS for approx. 10 min each time.

In a further preferred embodiment of the present invention, an increase in the resistance in the process according to the invention is attained in that
a) the expression of at least one callose synthase is reduced;
b) the stability of at least one callose synthase or of the mRNA molecules corresponding to this callose synthase is reduced;
c) the activity of at least one callose synthase is reduced;
d) the transcription of at least one of the genes coding for a callose synthase is reduced by expression of an endogenous or artificial transcription factor; or
e) an exogenous factor reducing the callose synthase activity is added to the nutrient or to the medium.

Gene expression and expression are to be used synonymously and mean the implementation of the information which is stored in a nucleic acid molecule. The reduction of the expression of a callose synthase gene thus comprises the reduction of the quantity of polypeptide of this callose synthase polypeptide, of the callose synthase activity or the callose synthase function. The reduction of the gene expression of a callose synthase gene can be effected in many ways, for example by one of the methods presented below.

“Reduction”, “decrease” or “decreasing” are to be broadly interpreted in connection with a callose synthase polypeptide, a callose synthase activity or callose synthase function and comprises the partial or essentially complete inhibition or blocking, based on different cell biology mechanisms, of the functionality of a callose synthase polypeptide in a plant or a part, tissue, organ, cells or seeds derived therefrom, based on various cell biological mechanisms.

A decrease in the sense of the invention also includes a quantitative diminution of a callose synthase polypeptide down to an essentially complete lack of the callose synthase polypeptide (i.e. lack of detectability of callose synthase activity or callose synthase function or lack of immunological detectability of the callose synthase polypeptide and also reduced callose deposits as a result of a pathogen attack). Here the expression of a certain callose synthase polypeptide or the callose synthase activity or callose synthase function in a cell or an organism is reduced preferably by more than 50%, particularly preferably by more than 80%, very particularly preferably by more than 90%, in comparison to the wild type of the same genus and species (“control plant”) on which this process was not used, under otherwise the same boundary conditions (such as for example cultivation conditions, age of the plant, etc.).

According to the invention, various strategies are described for the reduction of the expression of a callose synthase polypeptide, the callose synthase activity or callose synthase function. The skilled person recognizes that a range of further methods are available, in order to influence the expression of a callose synthase polypeptide, the callose synthase activity or the callose synthase function in a desired manner.

In one embodiment, in the process according to the invention a reduction in the callose synthase activity is attained by application of at least one process from the group selected from:
a) the introduction of a nucleic acid molecule coding for ribonucleic acid molecules suitable for the formation of double-stranded ribonucleic acid molecules (dsRNA), where the sense strand of the dsRNA molecule displays at least a homology of 30% to the nucleic acid molecule according to the invention, for example to one of the nucleic acid molecules according to SEQ. ID No: 1, 3, 5, 7, 9, 12, 14, 16, 18, 20, 22, 24, 26, 28, 30, 32 and/or 34 or comprises a fragment of at least 17 base pairs, which displays at least a 50% homology to a nucleic acid molecule according to the invention, for example according to SEQ. ID No: 1, 3, 5, 7, 9, 12, 14, 16, 18, 20, 22, 24, 26, 28, 30, 32 and/or 34, or to a functional equivalent thereof, or of an expression cassette or expression cassettes ensuring the expression thereof.
b) The introduction of a nucleic acid molecule coding for an antisense ribonucleic acid molecule which displays at least a homology of 30% to the non-coding strand of one of the nucleic acid molecules according to the invention, for example to a nucleic acid molecule according to SEQ. ID No: 1, 3, 5, 7, 9, 12, 14, 16, 18, 20, 22, 24, 26, 28, 30, 32 and/or 34 or comprises a fragment of at least 15 base pairs, which displays at least a 50% homology to a non-coding strand of a nucleic acid molecule according to the invention, for example according to SEQ. ID No: 1, 3, 5, 7, 9, 12, 14, 16, 18, 20, 22, 24, 26, 28, 30, 32 and/or 34 or to a functional equivalent thereof. Those processes wherein the antisense nucleic acid sequence is directed against a callose synthase gene (i.e. genomic DNA sequences) or a callose synthase gene transcript (i.e. RNA sequences) are comprised. α-Anomeric nucleic acid sequences are also comprised.
c) The introduction of a ribozyme which specifically cleaves, e.g. catalytically, the ribonucleic acid molecules encoded by a nucleic acid molecule according to the invention, for example according to SEQ. ID No: 1, 3, 5, 7, 9, 12, 14, 16, 18, 20, 22, 24, 26, 28, 30, 32 and/or 34 or by functional equivalents thereof or of an expression cassette ensuring the expression thereof.
d) The introduction of an antisense nucleic acid molecule such as specified in b), combined with a ribozyme or of an expression cassette ensuring the expression thereof.
e) The introduction of nucleic acid molecules coding for sense ribonucleic acid molecules of a polypeptide according to the invention, for example according to the sequences SEQ ID No: 2, 4, 6, 8, 10, 11, 13, 15, 17, 19, 21, 23, 25, 27, 29, 31, 33 and/or 35 or for polypeptides which display at least a 40% homology to the amino acid sequence of a protein according to the invention, or is a functional equivalent thereof.
f) The introduction of a nucleic acid sequence coding for a dominant-negative polypeptide suitable for the suppression of the callose synthase activity or of an expression cassette ensuring the expression thereof.
g) The introduction of a factor which can specifically bind callose synthase polypeptides or the DNA or RNA molecules coding for these or of an expression cassette ensuring the expression thereof.
h) The introduction of a viral nucleic acid molecule which causes a degradation of mRNA molecules coding for callose synthases or of an expression cassette ensuring the expression thereof.
i) The introduction of a nucleic acid construct suitable for the induction of a homologous recombination on genes coding for callose synthases.
j) The introduction of one or more mutations into one or more genes coding for callose synthases for the creation of a loss of function (e.g. generation of stop codons, reading frame shifts, etc.).

Each of these individual processes can cause a reduction in the callose synthase expression, callose synthase activity or callose synthase function in the sense of the invention. Combined use is also conceivable. Further methods are known to the skilled person and can include the hindrance or inhibition of the processing of the callose synthase polypeptide, the transport of the callose synthase polypeptide or its mRNA, inhibition of ribosome attachment, inhibition of RNA splicing, induction of a callose synthase RNA-degrading enzyme and/or inhibition of translational elongation or termination.

A reduction in the callose synthase activity, function or quantity of polypeptide is preferably achieved by decreased expression of an endogenous callose synthase gene.

The individual preferred processes may be briefly described below:

a) Incorporation of a Double-Stranded Callose Synthase RNA Nucleic Acid Sequence (Callose Synthase dsRNA)
The process of gene regulation by means of double-stranded RNA (“double-stranded RNA interference”; dsRNAi) has been described many times in animal and plant organisms (e.g. Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). An efficient gene suppression can also be demonstrated in transient expression or after transient transformation for example as a result of a biolistic transformation (Schweizer P et al. (2000) Plant J 2000 24: 895-903). dsRNAi processes are based on the phenomenon that through simultaneous incorporation of complementary strand and counterstrand of a gene transcript, a highly efficient suppression of the expression of the corresponding gene is effected. The resulting phenotype is very similar to that of a corresponding knock-out mutant (Waterhouse P M et al. (1998) Proc Natl Acad Sci USA 95:13959-64).
The dsRNAi process has proved particularly efficient and advantageous in the reduction of callose synthase expression (WO 99/32619).
With reference to the double-stranded RNA molecules, callose synthase nucleic acid sequence preferably means one of the sequences according to SEQ. ID No: 1, 3, 5, 7, 9, 12, 14, 16, 18, 20, 22, 24, 26, 28, 30, 32 and/or 34, or sequences which are essentially identical thereto, preferably at least 50%, 60%, 70%, 80% or 90% or more, for example about 95%, 96%, 97%, 98% or 99% or more, or fragments thereof which are at least 17 base pairs long. “Essentially identical” means that the dsRNA sequence can also display insertions, deletions and individual point mutations in comparison to the callose synthase target sequence and nonetheless cause an efficient reduction in expression. In one embodiment, the homology according to the above definition is at least 50%, for example about 80%, or about 90%, or about 100% between the “sense” strand of an inhibitory dsRNA and a part segment of a callose synthase nucleic acid sequence (e.g. between the “antisense” strand and the complementary strand of a callose synthase nucleic acid sequence). The length of the part segment is about 17 bases or more, for example about 25 bases, or about 50 bases, about 100 bases, about 200 bases or about 300 bases. Alternatively, an “essentially identical” dsRNA can also be defined as a nucleic acid sequence which is capable of hybridizing under stringent conditions with a part of a callose synthase gene transcript.
The “antisense” RNA strand can also display insertions, deletions and individual point mutations in comparison to the complement of the “sense” RNA strand. Preferably, the homology is at least 80%, for example about 90%, or about 95%, or about 100% between the “antisense” RNA strand and the complement of the “sense” RNA strand.
“Part segment of the “sense” RNA transcript” of a nucleic acid molecule coding for a callose synthase polypeptide or a functional equivalent thereof means fragments of an RNA or mRNA transcribed from a nucleic acid molecule coding for a callose synthase polypeptide or a functional equivalent thereof preferably from a callose synthase gene. Here the fragments preferably have a sequence length of about 20 bases or more, for example about 50 bases, or about 100 bases, or about 200 bases, or about 500 bases. The complete transcribed RNA or mRNA is also included.
The dsRNA can consist of one or more strands of polymerized ribonucleotides. Further, modifications both of the sugar-phosphate skeleton and also of the nucleosides can also be present. For example, the phosphodiester bonds of the natural RNA can be modified to the extent that they comprise at least one nitrogen or sulfur hetero atom. Bases can be modified to the extent that the activity for example of adenosine deaminase is limited. Such and further modifications are described below in the processes for the stabilization of antisense RNA.
Naturally, in order to achieve the same purpose, several individual dsRNA molecules, which each comprise one of the ribonucleotide sequence segments defined above, can also be incorporated in the cell or the organism.
The dsRNA can be produced enzymatically or wholly or partly by chemical synthesis.
If the two strands of the dsRNA are to be brought together in a cell or plant, this can occur in various ways:
a) transformation of the cell or plant with a vector which comprises both expression cassettes,
b) Cotransformation of the cell or plant with two vectors, where one comprises the expression cassettes with the “sense” strand, and the other the expression cassettes with the “antisense” strand, and/or
c) Crossing of two plants, which were each transformed with one vector, where one comprises the expression cassettes with the “sense” strand, and the other the expression cassettes with the “antisense” strand.
The formation of the RNA duplex can be initiated either outside the cell or within it. As described in WO 99/53050, the dsRNA can also comprise a hairpin structure, in that “sense” and “antisense” strand are linked via a “linker” (for example an intron). The self-complementary dsRNA structures are preferable, since they only require the expression of one construct and always comprise the complementary strands in an equimolar ratio.
The expression cassettes coding for the “antisense” or “sense” strand of a dsRNA or for the self-complementary strand of the dsRNA are preferably inserted into a vector and stably inserted into the genome of a plant with the processes described below (for example with the use of selection markers), in order to ensure lasting expression of the dsRNA.
The dsRNA can be introduced with the use of a quantity which makes at least one copy per cell possible. Higher quantities (e.g. at least 5, 10, 100, 500 or 1000 copies per cell) can on occasion result in a more efficient decrease.
100% sequence identity between dsRNA and a callose synthase gene transcript or the gene transcript of a functionally equivalent gene is not absolutely necessary in order to cause an efficient decrease in the callose synthase expression. There is thus the advantage that the process is tolerant towards sequence deviations such as may be present as a result of genetic mutations, polymorphisms or evolutionary divergences. The high sequence homology between the callose synthase sequences from rice, maize and barley indicates a high degree of conservation of this polypeptide within plants, so that the expression of a dsRNA derived from one of the disclosed callose synthase sequences according to SEQ. ID No: 1, 3, 5, 7, 9, 12, 14, 16, 18, 20, 22, 24, 26, 28, 30, 32 or 34 should also have an advantageous effect in other plant species.
On account of the high homology between the individual callose synthase polypeptides and functional equivalents thereof, it is also possible to suppress the expression of other homologous callose synthase polypeptides and/or functional equivalents thereof of the same organism or even the expression of callose synthase polypeptides in other related species, with a single dsRNA, which was generated starting from a certain callose synthase sequence of one organism. For this purpose, the dsRNA preferably comprises sequence regions of callose synthase gene transcripts which correspond to conserved regions. Said conserved regions can easily be derived from sequence comparisons.
A dsRNA can be synthesized chemically or enzymatically. For this, cellular RNA polymerases or bacteriophage RNA polymerases (such as for example T3, T7 or SP6 RNA polymerase) can be used. Corresponding processes for in vitro expression of RNA have been described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). A dsRNA chemically or enzymatically synthesized in vitro can be entirely or partially purified from the reaction mixture for example by extraction, precipitation, electrophoresis, chromatography or combinations of these processes before introduction into a cell, tissue or organism. The dsRNA can be directly introduced into the cell or else also applied extracellularly (e.g. into the interstitial space).
Preferably however, the plant is stably transformed with an expression construct which carries out the expression of the dsRNA. Appropriate processes are described below.
   b) Incorporation of a Callose Synthase Antisense Nucleic Acid Sequence
Processes for the suppression of a certain polypeptide by prevention of the accumulation of its mRNA using the “antisense” technology have been described many times, also in plants (Sheehy et al. (1988) Proc Natl Acad Sci USA 85: 8805-8809; U.S. Pat. No. 4,801,340; Mol J N et al. (1990) FEBS Lett 268(2):427-430). The antisense nucleic acid molecule hybridizes or binds to the cellular mRNA and/or genomic DNA coding for the callose synthase target polypeptide to be suppressed. As a result, the transcription and/or translation of the target polypeptide is suppressed. The hybridization can occur in the conventional way via the formation of a stable duplex or, in the case of genomic DNA, through binding of the antisense nucleic acid molecule with the duplex of the genomic DNA through specific interaction in the large groove of the DNA helix.
An antisense nucleic acid molecule suitable for decreasing a callose synthase polypeptide can be derived with the use of the nucleic acid sequence coding for this polypeptide, for example the nucleic acid molecule according to the invention according to SEQ. ID No: 1, 3, 5, 7, 9, 12, 14, 16, 18, 20, 22, 24, 26, 28, 30, 32 and/or 34 or a nucleic acid molecule coding for a functional equivalent thereof, in accordance with the base pair rules of Watson and Crick. The antisense nucleic acid molecule can be complementary to the total transcribed mRNA of said polypeptide, be restricted to the coding region or consist only of an oligonucleotide which is complementary to a part of the coding or non-coding sequence of the mRNA. Thus the oligonucleotide can for example be complementary to the region which comprises the translation start for said polypeptide. Antisense nucleic acid molecules can have a length of for example 20, 25, 30, 35, 40, 45 or 50 nucleotides, but can also be longer and contain 100, 200, 500, 1000, 2000 or 5000 nucleotides. Antisense nucleic acid molecules can be recombinantly expressed or synthesized chemically or enzymatically with the use of processes known to the skilled person. In the chemical synthesis, natural or modified nucleotides can be used. Modified nucleotides can impart increased biochemical stability to the antisense nucleic acid molecule, and result in increased physical stability of the duplex formed from antisense nucleic acid sequence and sense target sequence. Phosphorothioate derivatives and acridine-substituted nucleotides such as 5-fluorouracil, 5-bromouracil, 5-chlorouracil, 5-iodouracil, hypoxanthine, xanthine, 4-acetylcytosine, 5-(carboxyhydroxy-methyl)uracil, 5-carboxymethylaminomethyl-2-thiouridine, 5-carboxymethylamino-methyluracil, dihydrouracil, β-D-galactosylqueosine, inosine, N6-isopentenyl-adenine, 1-methylguanine, 1-methylinosine, 2,2-dimethylguanine, 2-methyl-adenine, 2-methylguanine, 3-methylcytosine, 5-methylcytosine, N6-adenine, 7-methylguanine, 5-methylamino-methyluracil, 5-methoxyaminomethyl-2-thiouracil, β-D-mannosyl queosine, 5′-methoxycarboxymethyluracil, 5-methoxy-uracil, 2-methylthio-N6-isopentenyladenine, uracil-5-hydroxyacetic acid, pseudouracil, queosine, 2-thiocytosine, 5-methyl-2-thiouracil, 2-thiouracil, 4-thiouracil, 5-methyluracil, uracil-5-hydroxyacetic acid methyl ester, uracil-5-hydroxyacetic acid, 5-methyl-2-thiouracil, 3-(3-amino-3-N-2-carboxypropyl)uracil and 2,6-diaminopurine can for example be used.
In a further preferred embodiment, the expression of a callose synthase polypeptide can be inhibited by nucleic acid molecules which are complementary to the regulatory region of a callose synthase gene (e.g., a callose synthase promoter and/or enhancer) and form triple-helical structures with the DNA double helix present there, so that the transcription of the callose synthase gene is decreased. Analogous processes have been described (Helene C (1991) Anticancer Drug Res 6(6):569-84; Helene C et al. (1992) Ann NY Acad Sci 660:27-36; Maher L J (1992) Bioassays 14(12):807-815). In a further embodiment, the antisense nucleic acid molecule can be an α-anomeric nucleic acid. Such α-anomeric nucleic acid molecules form specific double-stranded hybrids with complementary RNA wherein, in contrast to the conventional β-nucleic acids, the two strands run parallel to one another (Gautier C et al. (1987) Nucleic Acids Res 15:6625-6641). Further, the antisense nucleic acid molecule can also contain 2′-O-methylribonucleotides (Inoue et al. (1987) Nucleic Acids Res 15:6131-6148) or chimeric RNA-DNA analogs (Inoue et al. (1987) FEBS Lett 215:327-330).
   c) Incorporation of a Ribozyme which Specifically Cleaves the Ribonucleic Acid Molecules Coding for Callose Synthases, for Example Catalytically.
Catalytic RNA molecules or ribozymes can be matched to any target RNA and cleave the phosphodiester skeleton at specific positions, as a result of which the target RNA is functionally deactivated (Tanner N K (1999) FEMS Microbiol Rev 23(3):257-275). The ribozyme is not itself modified thereby, but rather is capable of similarly cleaving further target RNA molecules, as a result of which it takes on the properties of an enzyme.
In this way, ribozymes (e.g. “hammerhead” ribozymes; Haselhoff and Gerlach (1988) Nature 334:585-591) can be used to cleave the mRNA of an enzyme to be suppressed, e.g. callose synthases, and to inhibit its translation. Processes for the expression of ribozymes for the reduction of certain polypeptides are described in (EP 0 291 533, EP 0 321 201, EP 0 360 257). Ribozyme expression in plant cells has also been described (Steinecke P et al. (1992) EMBO J 11(4):1525-1530; de Feyter R et al. (1996) Mol Gen Genet. 250(3):329-338). Ribozymes can be identified via a selection process from a library of different ribozymes (Bartel D and Szostak J W (1993) Science 261:1411-1418).
   d) Incorporation of a Callose Synthase Antisense Nucleic Acid Sequence Combined with a Ribozyme.
The antisense strategy described above can advantageously be coupled with a ribozyme process. The incorporation of ribozyme sequences into “antisense” RNAs imparts to precisely these “antisense” RNAs this enzyme-like, RNA-cleaving property, and thus increases their efficiency in the inactivation of the target RNA. The production and use of appropriate ribozyme “antisense” RNA molecules is for example described in Haseloff et al. (1988) Nature 334: 585-591.
The ribozyme technology can increase the efficiency of an antisense strategy. Suitable target sequences and ribozymes can for example be determined as described in “Steinecke P, Ribozymes, Methods in Cell Biology 50, Galbraith et al. Eds, Academic Press, Inc. (1995), pp. 449-460”, by secondary structure calculations of ribozyme and target RNA and by their interaction (Bayley C C et al. (1992) Plant Mol Biol. 18(2):353-361; Lloyd A M and Davis R W et al. (1994) Mol Gen Genet. 242(6):653-657). For example, derivatives of the Tetrahymena L-19 IVS RNA which display complementary regions to the mRNA of the callose synthase polypeptide to be suppressed can be constructed (see also U.S. Pat. No. 4,987,071 and U.S. Pat. No. 5,116,742).
   e) Incorporation of a Callose Synthase Sense Nucleic Acid Sequence for Induction of Cosuppression
The expression of a callose synthase nucleic acid sequence in sense orientation can lead to cosuppression of the corresponding homologous, endogenous gene. The expression of sense RNA with homology to an endogenous gene can decrease or eliminate the expression thereof, in the same way as has been described for antisense approaches (Jorgensen et al. (1996) Plant Mol Biol 31(5):957-973; Goring et al. (1991) Proc Natl Acad Sci USA 88:1770-1774; Smith et al. (1990) Mol Gen Genet 224:447-481; Napoli et al. (1990) Plant Cell 2:279-289; Van der Krol et al. (1990) Plant Cell 2:291-99). Also, the construct introduced can wholly or only partially represent the homologous gene to be decreased. The capacity for translation is not necessary. The application of this technology to plants is for example described in Napoli et al. (1990) The Plant Cell 2: 279-289 and in U.S. Pat. No. 5,034,323.
The cosuppression is preferably effected with the use of a sequence which is essentially identical to at least a part of the nucleic acid sequence coding for a callose synthase polypeptide or a functional equivalent thereof, for example of the nucleic acid molecule according to the invention, e.g. of the nucleic acid sequence according to SEQ. ID No: 1, 3, 5, 7, 9, 12, 14, 16, 18, 20, 22, 24, 26, 28, 30, 32 and/or 34 or of the nucleic acid sequence coding for a functional equivalent thereof.
   f) Incorporation of Nucleic Acid Sequences Coding for a Dominant-Negative Callose Synthase Polypeptide.
The activity of a callose synthase polypeptide can presumably also be realized by expression of a dominant-negative variant of this callose synthase polypeptide. Processes for the reduction of the function or activity of a polypeptide by coexpression of its dominant-negative form are well known to the skilled person (Lagna G and Hemmati-Brivanlou A (1998) Current Topics in Developmental Biology 36:75-98; Perlmutter R M and Alberola-IIa J (1996) Current Opinion in Immunology 8(2):285-90; Sheppard D (1994) American Journal of Respiratory Cell & Molecular Biology. 11(1):1-6; Herskowitz I (1987) Nature 329(6136):219-22).
A dominant-negative callose synthase variant can for example arise by alteration of amino acid residues which are a component of the catalytic center and as a result of whose mutation the polypeptide loses its activity. Preferable amino acid residues for mutation are those which are conserved in the callose synthase polypeptides of different organisms. Such conserved regions can for example be determined by computer-assisted comparison (“alignment”). These mutations for obtaining a dominant-negative callose synthase variant are preferably effected at the level of the nucleic acid sequence coding for callose synthase polypeptides. An appropriate mutation can for example be realized by PCR-mediated in vitro mutagenesis with the use of appropriate oligonucleotide primers, by means of which the desired mutation is introduced. For this, processes familiar to the skilled person are used. For example, the “LA PCR in vitro Mutagenesis Kit” (Takara Shuzo, Kyoto) can be used for this purpose.
   g) Incorporation of Factors Binding Callose Synthase Genes, RNAs or Polypeptide.
A decrease in callose synthase gene expression is also possible with specific DNA binding factors, e.g. with factors of the zinc finger transcription factor type. These factors attach themselves to the genomic sequence of the endogenous target gene, preferably in the regulatory regions and cause repression of the endogenous gene. The use of such a process enables the reduction of the expression of an endogenous callose synthase gene, without the need to manipulate its sequence by genetic engineering. Appropriate processes for the preparation of such factors have been described (Dreier B et al. (2001) J Biol Chem 276(31):29466-78; Dreier B et al. (2000) J Mol Biol 303(4):489-502; Beerli R R et al. (2000) Proc Natl Acad Sci USA 97 (4):1495-1500; Beerli R R et al. (2000) J Biol Chem 275(42):32617-32627; Segal D J and Barbas C F 3rd. (2000) Curr Opin Chem Biol 4(1):34-39; Kang J S and Kim J S (2000) J Biol Chem 275(12):8742-8748; Beerli R R et al. (1998) Proc Natl Acad Sci USA 95(25):14628-14633; Kim J S et al. (1997) Proc Natl Acad Sci USA 94(8):3616-3620; Klug A (1999) J Mol Biol 293(2):215-218; Tsai S Y et al. (1998) Adv Drug Deliv Rev 30(1-3):23-31; Mapp A K et al. (2000) Proc Natl Acad Sci USA 97(8):3930-3935; Sharrocks A D et al. (1997) Int J Biochem Cell Biol 29(12):1371-1387; Zhang L et al. (2000) J Biol Chem 275(43):33850-33860).
The selection of these factors can be effected with the use of a suitable piece of a callose synthase gene. Preferably, this segment lies in the area of the promoter region. For suppression of a gene, however, it can also lie in the area of the coding exon or intron. The appropriate sections are obtainable for the skilled person from the gene bank by database interrogation or, starting from a callose synthase cDNA, the gene whereof is not present in the gene bank, by scrutiny of a genomic library for corresponding genomic clones. The processes necessary for this are familiar to the skilled person.
Further, factors can be introduced into a cell which inhibit the callose synthase target polypeptide itself. The polypeptide-binding factors can for example be aptamers (Famulok M and Mayer G (1999) Curr Top Microbiol Immunol 243:123-36) or antibodies or antibody fragments.
The obtention of these factors is described and is known to the skilled person. For example, a cytoplasmic scFv antibody was used to modulate the activity of the phytochrome A protein in genetically modified tobacco plants (Owen M et al. (1992) Biotechnology (NY) 10(7):790-794; Franken E et al. (1997) Curr Opin Biotechnol 8(4):411-416; Whitelam (1996) Trend Plant Sci 1:286-272).
The gene expression can also be suppressed with tailor-made, low molecular weight synthetic compounds, for example of the polyamide type (Dervan P B and Bürli R W (1999) Current Opinion in Chemical Biology 3:688-693; Gottesfeld J M et al. (2000) Gene Expr 9(1-2):77-91). These oligomers consist of the building blocks 3-(dimethylamino)propylamine, N-methyl-3-hydroxypyrrole, N-methylimidazole and N-methylpyrrole and can be matched to any piece of double-stranded DNA so that they bind sequence-specifically into the large groove and block the expression of the gene sequences present there. Appropriate processes have been described (see inter alia Bremer R E et al. (2001) Bioorg Med Chem. 9(8):2093-103; Ansari A Z et al. (2001) Chem Biol. 8(6):583-92; Gottesfeld J M et al. (2001) J Mol Biol. 309(3):615-29; Wurtz N R et al. (2001) Org Lett 3(8):1201-3; Wang C C et al. (2001) Bioorg Med Chem 9(3):653-7; Urbach A R and Dervan P B (2001) Proc Natl Acad Sci USA 98(8):4343-8; Chiang S Y et al. (2000) J Biol Chem. 275(32):24246-54).
   h) Incorporation of Viral Nucleic Acid Molecules and Expression Constructs Causing Callose Synthase RNA Degradation.
The expression of callose synthase can also be effectively realized by induction of the specific callose synthase RNA degradation by the plant with the aid of a viral expression system (amplicon) (Angell, S M et al. (1999) Plant J. 20(3):357-362). These systems, also described as “VIGS” (viral induced gene silencing), incorporate nucleic acid sequences with homology to the transcripts to be suppressed into the plant by means of viral vectors. The transcription is thereupon switched off, presumably mediated by plant defense mechanisms against viruses. Appropriate techniques and processes have been described (Ratcliff F et al. (2001) Plant J 25(2):237-45; Fagard M and Vaucheret H (2000) Plant Mol Biol 43(2-3):285-93; Anandalakshmi R et al. (1998) Proc Natl Acad Sci USA 95(22):13079-84; Ruiz M T (1998) Plant Cell 10(6): 937-46).
The methods of dsRNAi, of cosuppression using sense RNA and the “VIGS” (“virus induced gene silencing”) are also described as “post-transcriptional gene silencing” (PTGS). PTGS processes are particularly advantageous since the requirements for homology between the endogenous gene to be suppressed and the transgenically expressed sense or dsRNA nucleic acid sequence are less than for example with a classical antisense approach. Appropriate homology criteria are mentioned in the description of the dsRNAI process and are generally transferable for PTGS processes or dominant-negative approaches. On account of the high degree of homology between the callose synthase polypeptides from maize, wheat, rice and barley, it can be concluded that there is a high degree of conservation of this polypeptide in plants. Thus, the expression of homologous callose synthase polypeptides in other species can probably also be effectively suppressed by the use of the callose synthase nucleic acid molecules from barley, maize or rice, without any absolute need for the isolation and structure elucidation of the callose synthase homologs occurring there. This considerably lightens the cost of the work.
   i) Incorporation of a Nucleic Acid Construct Suitable for the Induction of Homologous Recombination in Genes Coding for Callose Synthases, for Example for the Generation of Knockout Mutants.
For the preparation of a homologously recombinant organism with decreased callose synthase activity, for example a nucleic acid construct is used which contains at least a part of an endogenous callose synthase gene which is modified by a deletion, addition or substitution of at least one nucleotide in such a way that the functionality is decreased or entirely eliminated. The modification can also concern the regulatory elements (e.g. the promoter) of the gene, so that the coding sequence remains unchanged, but expression (transcription and/or translation) ceases and is decreased.
In conventional homologous recombination, the modified region is flanked at its 5′- and 3′-end by other nucleic acid sequences which must be of sufficient length to render the recombination possible. The length as a rule lies in a range from several hundred or more bases up to several kilobases (Thomas K R and Capecchi M R (1987) Cell 51:503; Strepp et al. (1998) Proc Natl Acad Sci USA 95(8):4368-4373). For the homologous recombination, the host organism, for example a plant, is transformed with the recombination construct using the process described below and successfully recombined clones are selected using for example resistance to an antibiotic or a herbicide.
   j) Introduction of Mutations into Endogenous Callose Synthase Genes to Create a Loss of Function (e.g. Generation of Stop Codons, Reading Frame Shifts, etc.)
Further suitable methods for decreasing the callose synthase activity are the introduction of nonsense mutations into endogenous callose synthase genes, for example by generation of knockout mutants using for example T-DNA mutagenesis (Koncz et al. (1992) Plant Mol Biol 20(5):963-976), ENU—(N-ethyl-N-nitrosourea)—mutagenesis or homologous recombination (Hohn B and Puchta (1999) H Proc Natl Acad Sci USA 96:8321-8323) or EMS mutagenesis (Birchler J A, Schwartz D. Biochem Genet. 1979 December; 17(11-12):1173-80; Hoffmann G R. Mutat Res. 1980 January; 75(1):63-129). Point mutations can also be created by means of DNA-RNA hybrid oligonucleotides, which are also known as “chimeraplasty” (Zhu et al. (2000) Nat Biotechnol 18(5):555-558, Cole-Strauss et al. (1999) Nucl Acids Res 27(5):1323-1330; Kmiec (1999) Gene therapy American Scientist 87(3):240-247).

“Mutations” in the sense of the present invention means the modification of the nucleic acid sequence of a gene variant in a plasmid or in the genome of an organism. Mutations can for example be caused as a result of errors during replication or by mutagens. The rate of spontaneous mutations in the cell genome of organisms is very low, nonetheless a large number of biological, chemical or physical mutagens are known to the well-informed skilled person.

Mutations comprise substitutions, additions or deletions of one or several nucleic acid residues. Substitutions are understood to mean the exchange of individual nucleic acid bases, a distinction being made between transitions (substitution of a purine base for a purine base or of a pyrimidine base for a pyrimidine base) and transversions (substitution of a purine base for a pyrimidine base (or vice versa)).

Additions or insertion are understood to mean the incorporation of additional nucleic acid residues into the DNA, during which shifts in the reading frame can occur. With such reading frame shifts, a distinction is made between “in frame” insertions/additions and “out of frame” insertions. With the “in-frame” insertions/additions, the reading frame is retained and a polypeptide enlarged by the number of the amino acids encoded by the inserted nucleic acids is formed. With “out of frame” insertions/additions, the original reading frame is lost and the formation of a complete and functional polypeptide is no longer possible.

Deletions describe the loss of one or several base pairs, which likewise lead to “in frame” or “out of frame” shifts in the reading frame, and the consequences associated therewith as regards the formation of an intact protein.

The mutagenic agents (mutagens) applicable for the creation of random or targeted mutations and the applicable methods and techniques are well known to the skilled person. Such methods and mutagens are for example described in A. M. van Harten [(1998), “Mutation breeding: theory and practical applications”, Cambridge University Press, Cambridge, UK], E Friedberg, G Walker, W Siede [(1995), “DNA Repair and Mutagenesis”, Blackwell Publishing], or K. Sankaranarayanan, J. M. Gentile, L. R. Ferguson [(2000) “Protocols in Mutagenesis”, Elsevier Health Sciences].

For the introduction of targeted mutations, common molecular biological methods and processes such as for example the vitro Mutagense Kits, LA PCR in vitro Mutagenesis Kit” (Takara Shuzo, Kyoto), or PCR mutageneses with the use of suitable primers can be used.

As already stated above, there are a large number of chemical, physical and biological mutagens.

Those cited below may be mentioned by way of example, but not restrictively.

Chemical mutagens can be classified on the basis of their mechanism of action. Thus there are base analogs (e.g. 5-bromouracil, 2-aminopurine), mono- and bifunctional alkylating agents (e.g. monofunctional such as ethyl methylsulfonate and dimethyl sulfate, or bifunctional such as dichloroethyl sulfite, mitomycin, nitrosoguanidine-dialkylnitrosamines and N-nitrosoguanidine derivatives) or intercalating substances (e.g. acridines, ethidium bromide).

Physical mutagens are for example ionizing radiation. Ionizing radiation consists of electromagnetic waves or particle beams which are capable of ionizing molecules, i.e. of removing electrons from these. The ions that remain are mostly very reactive, so that, if they are formed in living tissue, they can cause great damage, e.g. to the DNA and (at low intensity) thereby induce mutations. Examples of ionizing radiation are gamma radiation (photon energy of about one mega-electron volt MeV), X-rays (photon energy of several or many kilo-electron volts keV) or even ultraviolet light (UV light, photon energy of over 3.1 eV). UV light causes the formation of dimers between bases, the commonest here are thymidine dimers, through which mutations arise.

The classical creation of mutants by treatment of the seeds with mutagenic agents such as for example ethyl methylsulfonate (EMS) (Birchler J A, Schwartz D. Biochem Genet. 1979 December; 17(11-12):1173-80; Hoffmann G R. Mutat Res. 1980 January; 75(1):63-129) or ionizing radiation has been extended by the use of biological mutagens e.g. transposons (e.g. Tn5, Tn903, Tn916, Tn1000, Balcells et al., 1991, May B P et al. (2003) Proc Natl Acad Sci USA. September 30; 100(20):11541-6) or molecular biology methods such as mutagenesis by T-DNA insertion (Feldman, K. A. Plant J. 1:71-82. 1991, Koncz et al. (1992) Plant Mol Biol 20(5):963-976).

The use of chemical or biological mutagens for the creation of mutated gene variants is preferred. In the case of the chemical agents, the creation of mutants by the use of EMS (ethyl methylsulfonate) mutagenesis is particularly preferably mentioned. In the creation of mutants with the use of biological mutagens, T-DNA mutagenesis or transposon mutagenesis may be preferably mentioned.

Thus for example, those polypeptides which are obtained as a result of a mutation of a polypeptide according to the invention, for example according to SEQ. ID No.: 2, 4, 6, 8, 10, 11, 13, 15, 17, 19, 21, 23, 25, 27, 29, 31, 33 and/or 35 can also be used for the process according to the invention.

All substances and compounds which directly or indirectly cause a reduction in the quantity of polypeptide, quantity of RNA, gene activity or polypeptide activity of a callose synthase polypeptide may thus be summarized under the term “anti-callose synthase compounds”. The term “anti-callose synthase compound” explicitly includes the nucleic acid sequences, peptides, proteins or other factors used in the processes described above.

In a further preferred embodiment of the present invention, an increase in resistance against pathogens from the Pucciniaceae, Mycosphaerellaceae and Hypocreaceae families in a monocotyledonous plant, or an organ, tissue or a cell thereof is attained by:
a) introduction of a recombinant expression cassette comprising an “anti-callose synthase compound” in functional linkage with a promoter active in plants, into a plant cell;
b) regeneration of the plant from the plant cell, and
c) expression of said “anti-callose synthase compound” in a quantity and for a time sufficient to create or to increase a pathogen resistance in said plant.

“Transgenic” means for example with regard to a nucleic acid sequence, an expression cassette or a vector comprising said nucleic acid sequence or an organism transformed with said nucleic acid sequence, expression cassette or vector, all those constructs or organisms that have come into existence through genetic engineering methods wherein either
a) the callose synthase nucleic acid sequence, or
b) a genetic control sequence, for example a promoter, functionally linked with the callose synthase nucleic acid sequence, or
c) (a) and (b)
   are not located in their natural genetic environment or have been modified by genetic engineering methods, where for example the modification can be a substitutions, additions, deletions, or insertions of one or several nucleotide residues. Natural genetic environment means the natural chromosomal locus in the source organism or the occurrence in a genome library. In the case of a genome library, the natural genetic environment of the nucleic acid sequence is preferably still at least partially retained. The environment flanks the nucleic acid sequence on at least one side and has a sequence length of at least 50 bp, preferably at least 500 bp, particularly preferably at least 1000 bp, and very particularly preferably at least 5000 bp. A naturally occurring expression cassette, for example the naturally occurring combination of the callose synthase promoter with the corresponding callose synthase gene, becomes a transgenic expression cassette, when this is modified by non-natural, synthetic (“artificial”) processes such as for example mutagenesis. Appropriate processes have been described (U.S. Pat. No. 5,565,350; WO 00/15815).

In the context of the invention “incorporation” comprises all process which are suitable for introducing an “anti-callose synthase compound”, directly or indirectly, into a plant or a cell, compartment, tissue, organ or seed thereof, or generating it there. Direct and indirect processes are comprised. The incorporation can result in a temporary (transient) or else also a lasting (stable) presence of an “anti-callose synthase compound” (for example a dsRNA).

In accordance with the diverse nature of the approaches described above, the “anti-callose synthase compound” can exert its function directly (for example by insertion into an endogenous callose synthase gene). The function can however also be exerted indirectly after transcription into an RNA (for example in antisense approaches) or after transcription and translation into a protein (for example with binding factors). Both direct and also indirectly acting “anti-callose synthase compounds” are comprised according to the invention.

“Incorporation” for example comprises processes such as transfection, transduction or transformation.

“Anti-callose synthase compound” thus for example also comprises recombinant expression constructs, which bring about expression (i.e. transcription and if necessary translation) for example of a callose synthase dsRNA or a callose synthase “antisense” RNA, preferably in a plant or a part, tissue, organ or seed thereof.

In said expression constructs/expression cassettes there is a nucleic acid molecule, the expression (transcription and if necessary translation) whereof generates an “anti-callose synthase compound”, preferably in functional linkage with at least one genetic control element (for example a promoter), which ensures expression in plants. If the expression construct is to be introduced directly into the plant and the “anti-callose synthase compound” (for example the callose synthase dsRNA) generated there in plantae, then plant-specific genetic control elements (for example promoters) are preferable. The “anti-callose synthase compound” can however also be generated in other organisms or in vitro and then introduced into the plant. In this, all prokaryotic or eukaryotic genetic control elements (for example promoters) which allow its expression in the particular plant selected for the preparation are preferable.

A functional linkage is understood to mean for example the sequential arrangement of a promoter or with the nucleic acid sequence to be expressed (for example an “anti-callose synthase compound”) and if necessary further regulatory elements such as for example a terminator in such a manner that each of the regulatory elements can fulfill its function in the transgenic expression of the nucleic acid sequence, depending on the arrangement of the nucleic acid sequences into sense or anti-sense RNA. For this, a direct linkage in the chemical sense is not absolutely necessary. Genetic control sequences, such as for example enhancer sequences, can also exert their function on the target sequence from more distant positions or even from other DNA molecules. Arrangements wherein the nucleic acid sequence to be transgenically expressed is positioned behind the sequence functioning as the promoter, so that both sequences are covalently bound together, are preferred. Here the distance between the promoter sequence and the nucleic acid sequence to be transgenically expressed is preferably less than 200 base pairs, particularly preferably smaller than 100 base pairs, very particularly preferably smaller than 50 base pairs.

The preparation of a functional linkage and also the preparation of an expression cassette can be effected by common recombination and cloning techniques, as for example described in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, further sequences can also be positioned between the two sequences, which for example have the function of a linker with defined restriction enzyme cleavage sites or a signal peptide. The insertion of sequences can also result in the expression of fusion proteins. Preferably, the expression cassette, consisting of a combination of promoter and nucleic acid sequence to be expressed, can be present integrated in a vector and be inserted into a plant genome for example by transformation.

An expression cassette should however also be understood to mean those constructs wherein a promoter is placed behind an endogenous callose synthase gene, for example by a homologous recombination, and the decrease in a callose synthase polypeptide according to the invention is effected by expression of an antisense callose synthase RNA. Analogously, an “anti-callose synthase compound” (for example a nucleic acid sequence coding for a callose synthase dsRNA or a callose synthase antisense RNA) can also be placed behind an endogenous promoter in such a manner that the same effect arises. Both approaches result in expression cassettes in the sense of the invention.

Plant-specific promoters means essentially any promoter which can control the expression of genes, in particular foreign genes, in plants or plant parts, cells, tissues or cultures. Here, the expression can for example be constitutive, inducible or development-dependent.

Preferred are:

a) Constitutive Promoters

Preferred are vectors which enable constitutive expression in plants (Benfey et al. (1989) EMBO J 8:2195-2202). “Constitutive” promoter means promoters, which ensure expression in many, preferably all, tissues over a considerable period of the plant development, preferably at all times in the plant development. Preferably, in particular a plant promoter or a promoter which derives from a plant virus is used. Particularly preferable is the promoter of the 35S transcript of the CaMV cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or the 19S CaMV promoter (U.S. Pat. No. 5,352,605; WO 84/02913; Benfey et al. (1989) EMBO J 8:2195-2202). A further suitable constitutive promoter is the “rubisco small subunit (SSU)”-promoter (U.S. Pat. No. 4,962,028), the promoter of nopalin synthase from Agrobacterium, the TR double promoter, the OCS (octopin synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits or the promoter of a proline-rich protein from wheat (WO 91/13991), and further promoters of genes, the constitutive expression whereof in plants is known to the skilled person. Particularly preferable as a constitutive promoter is the promoter of the nitrilase-1 (nit1) gene from A. thaliana (GenBank Acc.-No.: Y07648.2, nucleotides 2456-4340, Hillebrand et al. (1996) Gene 170:197-200).

b) Tissue-Specific Promoters

In one embodiment, promoters with specificities for the anthers, ovaries, flowers, leaves, stems, roots and seeds are used.

### Seed-Specific Promoters

such as for example the promoter of phaseolin (U.S. Pat. No. 5,504,200; Bustos M M et al. (1989) Plant Cell 1(9):839-53), of the 2S albumin gene (Joseffson L G et al. (1987) J Biol Chem 262:12196-12201), of legumin (Shirsat A et al. (1989) Mol Gen Genet 215(2): 326-331), of USP (unknown seed protein; Baumlein H et al. (1991) Mol Gen Genet 225(3):459-67), of the napin gene (U.S. Pat. No. 5,608,152; Stalberg K et al. (1996) L Planta 199:515-519), of saccharose binding protein (WO 00/26388) or the legumin B4 promoter (LeB4; Bäumlein H et al. (1991) Mol Gen Genet 225: 121-128; Baeumlein et al. (1992) Plant Journal 2(2):233-9; Fiedler U et al. (1995) Biotechnology (NY) 13(10):1090f), the oleosin promoter from Arabidopsis (WO 98/45461), and the Bce4 promoter from Brassica (WO 91/13980). Further suitable seed-specific promoters are those of the genes coding for “High Molecular Weight Glutenin” (HMWG), gliadin, branching enzyme, ADP glucose pyrophosphatase (AGPase) or starch synthase. Also preferred are promoters which allow seed-specific expression in monocotyledons such as maize, barley, wheat, rye, rice etc. The promoter of the Ipt2 or Ipt1 gene (WO 95/15389, WO 95/23230) or the promoters described in WO 99/16890 (promoters of the hordein gene, the glutelin gene, the oryzin gene, the prolamine gene, the gliadin gene, the zein gene, the kasirin gene or the secalin gene) can advantageously be used.

Tuber, storage root or root-specific promoters such as for example the patatin promoter class I (B33), and the promoter of the cathepsin D inhibitor from potatoes.

### Leaf-Specific Promoters

such as the promoter of the cytosol FBPase from potatoes (WO 97/05900), the SSU promoter (small subunit) of rubisco (Ribulose-1,5-bisphosphatecarboxylase) or the ST-LSI promoter from potatoes (Stockhaus et al. (1989) EMBO J 8:2445-2451). Epidermis-specific promoters, such as for example the promoter of the OXLP gene (“oxalate oxidase-like protein”; Wei et al. (1998) Plant Mol. Biol. 36:101-112).

### Flower-Specific Promoters

such as for example the phytoen synthase promoter (WO 92/16635) or the promoter of the P-rr gene (WO 98/22593).

### Anther-Specific Promoters

such as the 5126 promoter (U.S. Pat. No. 5,689,049, U.S. Pat. No. 5,689,051), the glob-I promoter and the γ-zein promoter.

c) Chemically Inducible Promoters

The expression cassettes can also comprise a chemically inducible promoter (Review article: Gatz et al. (1997) Annu. Rev. Plant Physiol Plant Mol Biol 48:89-108), through which the expression of the exogenous gene in the plant can be controlled at a defined time point. Such promoters, such as for example the PRP1 promoter (Ward et al. (1993) Plant Mol Biol 22:361-366), salicylic acid-inducible promoter (WO 95/19443), a benzenesulfonamide-inducible promoter (EP 0 388 186), a tetracycline-inducible promoter (Gatz et al. (1992) Plant J 2:397-404), an abscissic acid-inducible promoter (EP 0 335 528) or an ethanol- or cyclohexanone-inducible promoter (WO 93/21334) can likewise be used. Thus for example the expression of a molecule reducing or inhibiting the callose synthase activity, such as for example the dsRNA, ribozymes, antisense nucleic acid molecules etc. enumerated above can be induced at suitable time points.

d) Stress- or Pathogen-Inducible Promoters

Very particularly advantageous is the use of inducible promoters for the expression of the RNAi constructs used for the reduction of the callose synthase quantity of polypeptide, activity or function, which for example with the use of pathogen-inducible promoters enables expression only in case of need (i.e. pathogen attack).

Hence in the process according to the invention, in one embodiment active promoters, which are pathogen-inducible promoters are used in plants.

Pathogen-inducible promoters include the promoters of genes which are induced as a result of pathogen attack, such as for example genes of PR proteins, SAR proteins, β-1,3-glucanase, chitinase etc. (for example Redolfi et al. (1983) Neth J Plant Pathol 89:245-254; Uknes, et al. (1992) Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989).

Also comprised are injury-inducible promoters such as that of the pinII gene (Ryan (1990) Ann Rev Phytopath 28:425-449; Duan et al. (1996) Nat Biotech 14:494-498), the wun1 and wun2-gene (U.S. Pat. No. 5,428,148), the win1 and win2 gene (Stanford et al. (1989) Mol Gen Genet 215:200-208), systemin (McGurl et al. (1992) Science 225:1570-1573), the WIP1 gene (Rohmeier et al. (1993) Plant Mol Biol 22:783-792; Eckelkamp et al. (1993) FEBS Letters 323:73-76), the MPI gene (Corderok et al. (1994) Plant J 6(2):141-150) and the like.

The PR gene family represents a source for further pathogen-inducible promoters. A range of elements in these promoters have proved to be advantageous. Thus, the region −364 to −288 in the promoter of PR-2d mediates salicylate-specificity (Buchel et al. (1996) Plant Mol Biol 30, 493-504). The sequence 5′-TCATCTTCTT-3′ occurs repeatedly in the promoter of the barley β-1,3-glucanase and in more than 30 other stress-induced genes. In tobacco, this region binds a nuclear protein the abundance of which is increased by salicylate. The PR-1 promoters from tobacco and Arabidopsis (EP-A 0 332 104, WO 98/03536) are also suitable as pathogen-inducible promoters. Preferably, since particularly specifically pathogen-induced, are the “acidic PR-5”-(aPR5) promoters from barley (Schweizer et al. (1997) Plant Physiol 114:79-88) and wheat (Rebmann et al. (1991) Plant Mol Biol 16:329-331). aPR5 proteins accumulate in approx. 4 to 6 hours after pathogen attack and display only very slight background expression (WO 99/66057). One approach for achieving increased pathogen-induced specificity is the preparation of synthetic promoters from combinations of known pathogen-responsive elements (Rushton et al. (2002) Plant Cell 14, 749-762; WO 00/01830; WO 99/66057). Other pathogen-inducible promoters from various species are known to the skilled person (EP-A 1 165 794; EP-A 1 062 356; EP-A 1 041 148; EP-A 1 032 684).

Other pathogen-inducible promoters include the flax Fis1 promoter (WO 96/34949), the Vstl promoter (Schubert et al. (1997) Plant Mol Biol 34:417-426) and the EAS4 sesquiterpene cyclase promoter from tobacco (U.S. Pat. No. 6,100,451).

Further, promoters which are induced by biotic or abiotic stress, such as for example the pathogen-inducible promoter of the PRP1 gene (or gst1 promoter) e.g. from potatoes (WO 96/28561; Ward et al. (1993) Plant Mol Biol 22:361-366), the heat-inducible hsp70 or hsp80 promoter from tomatoes (U.S. Pat. No. 5,187,267), the cold-inducible alpha-amylase promoter from the potato (WO 96/12814), the light-inducible PPDK promoter or the injury-induced pinII promoter (EP-A 0 375 091), are preferred.

e) Mesophyllic Tissue-Specific Promoters

In the process according to the invention, in one embodiment mesophyllic tissue-specific promoters such as for example the promoter of the wheat germin 9f-3.8 gene (GenBank Acc.-No.: M63224) or the barley GerA promoter (WO 02/057412) are used. Said promoters are particularly advantageous since they are both mesophyllic tissue-specific and pathogen-inducible. Also suitable is the mesophyllic tissue-specific Arabidopsis CAB-2 promoter (GenBank Acc.-No.: X15222), and the Zea mays PPCZm1 promoter (GenBank Acc.-No.: X63869) or homologs thereof. Mesophyllic tissue-specific means a restriction of the transcription of a gene through the specific interaction of cis elements present in the promoter sequence, and transcription factors binding thereto, to as few as possible plant tissues comprising the mesophyllic tissue, and preferably transcription restricted to the mesophyllic tissue is meant.

f) Development-Dependent Promoters

Further suitable promoters are for example fruit ripening-specific promoters, such as for example the fruit ripening-specific promoter from the tomato (WO 94/21794, EP 409 625). Development-dependent promoters to some extent includes the tissue-specific promoters, since the formation of individual tissue naturally takes place as a function of development.

Constitutive, and leaf and/or stem-specific, pathogen-inducible, root-specific, mesophyllic tissue-specific promoters are particularly preferable, constitutive, pathogen-inducible, mesophyllic tissue-specific and root-specific promoters being most preferable.

Further, other promoters, which enable expression in other plant tissues or in other organisms, such as for example E. coli bacteria, can be functionally linked to the nucleic acid sequence to be expressed. As plant promoters, in principle all the promoters described above are possible.

Further promoters suitable for expression in plants have been described (Rogers et al. (1987) Meth in Enzymol 153:253-277; Schardl et al. (1987) Gene 61:1-11; Berger et al. (1989) Proc Natl Acad Sci USA 86:8402-8406).

The nucleic acid sequences comprised in the expression cassettes or vectors according to the invention can be functionally linked to other genetic control sequences as well as a promoter. The term genetic control sequences should be broadly understood and means all sequences, which have an effect on the creation or the function of the expression cassette according to the invention. Genetic control sequences for example modify transcription and translation in prokaryotic or eukaryotic organisms. Preferably, the expression cassettes according to the invention comprise a promoter with one of the specificity described above 5′ upstream from the particular nucleic acid sequence to be transgenically expressed, and a terminator sequence as an additional genetic control sequence 3′ downstream, and if necessary further normal regulatory elements, these in each case being functionally linked to the nucleic acid sequence to be transgenically expressed.

Genetic control sequences also comprise further promoters, promoter elements or minimal promoters, which can modify the expression-controlling properties. Thus for example, by means of genetic control sequences, the tissue-specific expression can also take place dependent on certain stress factors. Analogous elements have for example been described for water stress, abscissic acid (Lam E and Chua N H, J Biol Chem 1991; 266(26): 17131-17135) and heat stress (Schoffl F et al., Molecular & General Genetics 217(2-3):246-53, 1989).

In principle, all natural promoters with their regulatory sequences such as those mentioned above can be used for the process according to the invention. Moreover, synthetic promoters can also advantageously be used.

Genetic control sequences further also comprise the 5′-untranslated regions, introns or non-coding 3′-region of genes such as for example the actin-1 intron, or the Adh1-S introns 1, 2 and 6 (in general: The Maize Handbook, Chapter 116, Freeling and Walbot, Eds., Springer, New York (1994)). It has been shown that these can have a significant function in the regulation of gene expression. Thus it has been shown that 5′ untranslated sequences can amplify the transient expression of heterologous genes. As examples of translation amplifiers, the 5′ leader sequence from the tobacco mosaic virus (Gallie et al. (1987) Nucl Acids Res 15:8693-8711) and the like can be mentioned. The can also promote tissue-specificity (Rouster J et al. (1998) Plant J 15:435-440).

The expression cassette can advantageously comprise one or several so-called “enhancer sequences” functionally linked with the promoter, which enable increased transgenic expression of the nucleic acid sequence. Additional advantageous sequences, such as further regulatory elements or terminators, can also be inserted at the 3′ end of the nucleic acid sequence to be transgenically expressed. The nucleic acid sequences to be transgenically expressed can be comprised in the gene construct in one or several copies.

Polyadenylation signals suitable as control sequences are plant polyadenylation signals, preferably those which essentially correspond to T-DNA polyadenylation signals from Agrobacterium tumefaciens, in particular gene 3 of the T-DNA (octopin synthase) of the Ti plasmid pTiACHS (Gielen et al. (1984) EMBO J 3:835 ff) or functional equivalents thereof. Examples of particularly suitable terminator sequences are the OCS (octopin synthase) terminator and the NOS (nopalin synthase) terminator.

Also to be understood as control sequences are those which enable a homologous recombination or insertion into the genome of a host organism or which allow removal from the genome. In the homologous recombination, for example the natural promoter of a certain gene can be exchanged for a promoter with specificities for the embryonic epidermis and/or the flower. An expression cassette and the vectors derived therefrom can comprise further functional elements. The term functional element should be broadly understood and means all elements which have an effect on production, reproduction or function of the expression cassettes, vectors or transgenic organisms according to the invention. By way of example, but not restrictively, the following may be mentioned:
a) Selection markers which impart resistance against a metabolic inhibitor such as 2-desoxyglucose-6-phosphate (WO 98/45456), antibiotics or biocides, preferably herbicides, such as for example kanamycin, G 418, bleomycin, hygromycin, or phosphinothricin etc. Particularly preferable selection markers are those which impart resistance against herbicides. By way of example, DNA sequences which code for phosphinothricin acetyltransferases (PAT) and inactivate glutamine synthase inhibitors (bar and pat gene), 5-enolpyruvylshikimate-3-phosphate synthase genes (EPSP synthase genes), which impart resistance against Glyphosate® (N-(phosphonomethyl)glycine), the gox gene coding for the Glyphosate®-degrading enzyme (glyphosate oxidoreductase), the deh gene (coding for a dehalogenase, which inactivates Dalapon), sulfonylurea and imidazolinone inactivating acetolactate synthases and bxn genes, which code for nitrilase enzymes degrading Bromoxynil, the aasa gene, which imparts resistance against the antibiotic apectinomycin, the streptomycin phosphotransferase (SPT) gene, which ensures resistance against streptomycin, the neomycin phosphotransferase (NPTII) gene, which imparts resistance against kanamycin or geneticidin, the hygromycin phosphotransferase (HPT) gene, which mediates resistance against hygromycin, and the acetolactate synthase gene (ALS), which imparts resistance against sulfonylurea herbicides (e.g. mutated ALS variants with for example the S4 and/or Hra mutation) may be mentioned.
b) Reporter genes, which code for easily quantifiable proteins and through their own color or enzyme activity ensure an assessment of the transformation efficiency or the expression site or time point. Very particularly preferable here are reporter proteins (Schenborn E, Groskreutz D. Mol Biotechnol. 1999; 13(1):29-44) such as the “green fluorescence protein” (GFP) (Sheen et al. (1995) Plant Journal 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228; Chui W L et al. (1996) Curr Biol 6:325-330; Leffel S M et al. (1997) Biotechniques. 23(5):912-8), chloramphenicol transferase, a luciferase (Ow et al. (1986) Science 234:856-859; Millar et al. (1992) Plant Mol Biol Rep 10:324-414), the aequorin gene (Prasher et al. (1985) Biochem Biophys Res Commun 126(3):1259-1268), the β-galactosidase, R-locus gene (encode a protein, which regulates the production of anthocyanin pigments (red coloration) in plant tissue and thus enables a direct analysis of the promoter activity without addition of supplementary additives or chromogenic substrates; Dellaporta et al., In: Chromosome Structure and Function: Impact of New Concepts, 18th Stadler Genetics Symposium, 11:263-282, 1988), and β-glucuronidase is very particularly preferable (Jefferson et al., EMBO J. 1987, 6, 3901-3907).
c) Replication origins which ensure replication of the expression cassettes or vectors according to the invention in for example E. coli. By way of example, ORI (origin of DNA replication), the pBR322 ori or the P15A ori (Sambrook et al.: Molecular Cloning. A Laboratory Manual, 2^{nd} ed. Cold Spring Harbor Laboratory Press, Cold Spring Harbor, N.Y., 1989) may be mentioned.
d) Elements which are necessary for an Agrobacterium-mediated plant transformation, such as for example the right or left boundary of the T-DNA or the vir region.

For the selection of successfully transformed cells, it is as a rule necessary also to introduce a selectable marker which imparts to the successfully transformed cells a resistance against a biocide (for example a herbicide), a metabolic inhibitor such as 2-desoxyglucose-6-phosphate (WO 98/45456) or an antibiotic. The selection marker allows the selection of the transformed from untransformed cells (McCormick et al. (1986) Plant Cell Reports 5:81-84).

The introduction of an expression cassette according to the invention into an organism or cells, tissues, organs, parts or seeds thereof (preferably in plants or plant cells, tissue, organs, parts or seeds), can advantageously be performed with the use of vectors wherein the expression cassettes are comprised. The expression cassette can be introduced into the vector (for example a plasmid) via a suitable restriction cleavage site. The resulting plasmid is firstly introduced into E. coli. Correctly transformed E. coli are selected, grown and the recombinant plasmid obtained by methods familiar to the skilled person. Restriction analysis and sequencing can be used for checking the cloning step.

Vectors can for example be plasmids, cosmids, phages, viruses or also Agrobacteria. In an advantageous embodiment, the introduction of the expression cassette is effected by means of plasmid vectors. Vectors which enable stable integration of the expression cassette into the host genome are preferred.

The preparation of a transformed organism (or of a transformed cell) requires that the corresponding DNA molecules and hence the RNA molecules or proteins formed as a result of the gene expression thereof are introduced into the appropriate host cell.

For this procedure, which is described as transformation (or transduction or transfection), a large number of methods are available (Keown et al. (1990) Methods in Enzymology 185:527-537). Thus for example the DNA or RNA can be directly introduced by microinjection or by bombardment with DNA-coated microparticles. Also, the cell can be chemically permeabilized, for example with polyethylene glycol, so that the DNA can get into the cell by diffusion. The DNA can also be effected by protoplast fusion with other DNA-containing units such as minicells, cells, lysosomes or liposomes. Electroporation is a further suitable method for the introduction of DNA, in which the cells are reversibly permeabilized by an electrical impulse. Appropriate processes have been described (for example in Bilang et al. (1991) Gene 100:247-250; Scheid et al. (1991) Mol Gen Genet 228:104-112; Guerche et al. (1987) Plant Science 52:111-116; Neuhause et al. (1987) Theor Appl Genet 75:30-36; Klein et al. (1987) Nature 327:70-73; Howell et al. (1980) Science 208:1265; Horsch et al. (1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.) Academic Press Inc. (1989)).

In plants also, the described methods for the transformation and regeneration of plants from plant tissues or plant cells are used for transient or stable transformation. Suitable methods are in particular protoplast transformation by polyethylene glycol-induced DNA uptake, the biolistic process with the gene cannon, the so-called “particle bombardment” method, electroporation, the incubation of dry embryos in DNA-containing solution and microinjection.

As well as these “direct” transformation techniques, a transformation can also be performed by bacterial infection with Agrobacterium tumefaciens or Agrobacterium rhizogenes. The processes have for example been described in Horsch R B et al. (1985) Science 225: 1229f).

If Agrobacteria are used, the expression cassette must be integrated into special plasmids, either in an intermediate vector (shuttle or intermediate vector) or a binary vector. If a Ti or Ri plasmid is used for the transformation, at least the right boundary, but mostly the right and left boundary of the Ti or Ri plasmid T-DNA must be bound as a flanking region with the expression cassette to be introduced.

Binary vectors are preferably used. Binary vectors can replicate both in E. coli and also in Agrobacterium. As a rule they comprise a selection marker gene and a linker or polylinker flanked by the right and left T-DNA boundary sequence. They can be directly transformed into Agrobacterium (Holsters et al. (1978) Mol Gen Genet 163:181-187). The selection marker gene allows selection of transformed Agrobacteria and is for example the nptII gene, which imparts resistance against kanamycin. The Agrobacterium functioning as the host organism in this case should already contain a plasmid with the vir region. This is necessary for the transfer of the T-DNA to the plant cell. An Agrobacterium thus transformed can be used for the transformation of plant cells. The use of T-DNA for the transformation of plant cells has been intensively studied and described (EP 120 516; Hoekema, In: The Binary Plant Vector System, Offsetdrukkerij Kanters B.V., Alblasserdam, Chapter V; An et al. (1985) EMBO J 4:277-287). Various binary vectors are known and some are commercially available such as for example pBI101.2 or pBIN19 (Clontech Laboratories, Inc. USA).

In the case of injection or electroporation of DNA or RNA into plant cells, no special requirements are set as to the plasmid used. Simple plasmids such as the pUC range can be used. If complete plants are to be regenerated from the transformed cells, an additional selectable marker gene must be present on the plasmid.

Stably transformed cells, i.e. those which comprise the introduced DNA integrated into the DNA of the host cell, can be selected from untransformed cells when a selectable marker is a component of the introduced DNA. For example, any gene which is able to impart resistance against antibiotics or herbicides (such as kanamycin, G 418, bleomycin, hygromycin or phosphinotricin etc.) (see above) can function as a marker. Transformed cells which express such a marker gene are capable of surviving in the presence of concentrations of a corresponding antibiotic or herbicide which kill an untransformed wild type. Examples are mentioned above and preferably comprise the bar gene, which imparts resistance against the herbicide phosphinotricin (Rathore K S et al. (1993) Plant Mol Biol 21(5):871-884), the nptII gene, which imparts resistance against kanamycin, the hpt gene, which imparts resistance against hygromycin, or the EPSP gene, which imparts resistance against the herbicide glyphosate. The selection marker allows the selection of transformed from untransformed cells (McCormick et al. (1986) Plant Cell Reports 5:81-84). The plants obtained can be grown and crossed in the normal way. Two or more generations should be cultivated in order to ensure that the genomic integration is stable and transmissible.

The processes mentioned above are for example described in Jenes B et al. (1993) Techniques for Gene Transfer, in: Transgenic Plants, Vol. 1, Engineering and Utilization, edited by S D Kung and R Wu, Academic Press, pp. 128-143 and in Potrykus (1991) Annu Rev Plant Physiol Plant Molec Biol 42:205-225). Preferably, the construct to be expressed is cloned into a vector with is suitable for transforming Agrobacterium tumefaciens, for example pBin19 (Bevan et al. (1984) Nucl Acids Res 12:8711f).

As soon as a transformed plant cell has been produced, a complete plant can be obtained with the use of processes well known to the skilled person. Here for example, callus cultures are the starting point. From these still undifferentiated cell masses, the formation of shoot and roots can be induced in a known manner. The shoots obtained can be planted out and grown.

Also well known to the skilled person are processes for regenerating plant parts and whole plants from plant cells. For example, processes described by Fennell et al. (1992) Plant Cell Rep. 11: 567-570; Stoeger et al (1995) Plant Cell Rep. 14:273-278; Jahne et al. (1994) Theor Appl Genet 89:525-533 are used for this.

The process according to the invention can advantageously be combined with other processes which cause a pathogen resistance (for example against insects, fungi, bacteria, nematodes, etc.), stress resistance or another improvement of the plant properties. Examples are inter alia mentioned in Dunwell J M, Transgenic approaches to crop improvement, J Exp Bot. 2000; 51 Spec No; page 487-96.

In a preferred embodiment, the reduction of the activity of a callose synthase is effected in a plant in combination with an increase in the activity of a Bax inhibitor-1 protein. This can for example be effected by expression of a nucleic acid sequence coding for a Bax inhibitor-1 protein, e.g. in the mesophyllic tissue and/or root tissue.

In the process according to the invention, the Bax inhibitor-1 proteins from Hordeum vulgare (SEQ ID No:37) or Nicotiana tabacum SEQ ID No: 39) are particularly preferable.

A further object of the invention relates to nucleic acid molecules, which include nucleic acid molecules coding for callose synthase polypeptides from barley, wheat and maize according to the polynucleotides SEQ. ID No: 3, 5, 7, 9, 12, 14, 16, 22, 24, 26, 28, 30, and/or 32, and the nucleic acid sequences complementary thereto, and the sequences derived by degeneration of the genetic code and the nucleic acid molecules coding for functional equivalents of the polypeptides according to SEQ. ID No.: 4, 6, 8, 10, 11, 13, 15, 17, 23, 25, 27, 29, 31 and/or 33, where the nucleic acid molecules do not consist of the SEQ ID No: 1, 18, 20 or 34.

A further object of the invention relates to the callose synthase polypeptide from barley, wheat, maize according to SEQ. ID No.: 4, 6, 8, 10, 11, 13, 15, 17, 23, 25, 27, 29, 31 or 33 or one which comprises these sequences, and functional equivalents thereof, which do not consist of the SEQ ID No: 2, 19, 21 or 35.

A further object of the invention relates to double-stranded RNA nucleic acid molecules (dsRNA molecules), which on introduction into a plant (or a cell, tissue, organ or seed derived therefrom) cause a decrease in a callose synthase, where the sense strand of said dsRNA molecule displays at least a homology of 30%, preferably at least 40%, 50%, 60%, 70% or 80%, particularly preferably at least 90%, very particularly preferably 100% to a nucleic acid molecule according to SEQ. ID No: 3, 5, 7, 9, 12, 14, 16, 22, 24, 26, 28, 30, and/or 32, or comprises a fragment of at least 17 base pairs, preferably at least 18, 19, 20, 21, 22, 23, 24, 25, 26, 27, 28, 29 or 30 base pairs, particularly preferably at least 40, 50, 60, 70, 80 or 90 base pairs, very particularly preferably at least 100, 200, 300 or 400 base pairs, most preferably of all at least 500, 600, 700, 800, 900 at least 1000 base pairs, and which displays at least a 50%, 60%, 70% or 80%, particularly preferably at least 90%, very particularly preferably 100% homology to a nucleic acid molecule according to SEQ. ID No: 3, 5, 7, 9, 12, 14, 16, 22, 24, 26, 28, 30, and/or 32, but do not correspond to SEQ ID No: 1, 18, 20 and 34.

The double-stranded structure can be formed from a single, self-complementary strand or from two complementary strands. In a particularly preferable embodiment, “sense” and “antisense” sequence are linked by a linking sequence (“linker”) and can for example form a hairpin structure. Very particularly preferably, the linking sequence can be an intron which is spliced out after synthesis of the dsRNA.

The nucleic acid sequence coding for a dsRNA can contain further elements, such as for example transcription termination signals or polyadenylation signals.

A further object of the invention relates to transgenic expression cassettes which contain one of the nucleic acid sequences according to the invention. In the transgenic expression cassettes according to the invention, the nucleic acid sequence coding for the callose synthase polypeptides from barley, wheat and maize is linked with at least one genetic control element according to the above definition in such a manner that the expression (transcription and if necessary translation) can be effected in any organism, preferably in monocotyledonous plants. Genetic control elements suitable for this are described above. The transgenic expression cassettes can also contain further functional elements according to the above definition.

Such expression cassettes for example contain a nucleic acid sequence according to the invention, e.g. one which is essentially identical to a nucleic acid molecule according to ID No: 3, 5, 7, 9, 12, 14, 16, 22, 24, 26, 28, 30, or 32, or fragment thereof according to the invention, where said nucleic acid sequence is preferably present in sense orientation or in antisense orientation to a promoter and thus can lead to expression of sense or antisense RNA, said promoter being a promoter active in plants, preferably one inducible by pathogen attack. According to the invention, transgenic vectors which contain said transgenic expression cassettes are also included.

Another object of the invention relates to plants which comprise mutations induced by natural processes or artificially in a nucleic acid molecule which comprises the nucleic acid sequence according to SEQ. ID No: 3, 5, 7, 9, 12, 14, 16, 22, 24, 26, 28, 30, or 32, which do not consist of the SEQ ID No: 1, 18, 20 and 34, where said mutation causes a decrease in the activity, function or quantity of polypeptide of a polypeptide encoded by the nucleic acid molecules according to SEQ. ID No: 3, 5, 7, 9, 12, 14, 16, 22, 24, 26, 28, 30, or 32. Plants which belong to the Poaceae family are preferred here, particularly preferred are plants selected from the plant genera Hordeum, Avena, Secale, Triticum, Sorghum, Zea, Saccharum and Oryza, very particularly preferably plants selected from the species Hordeum vulgare (barley), Triticum aestivum (wheat), Triticum aestivum subsp. spelta (spelt), Triticale, Avena sative (oats), Secale cereale (rye), Sorghum bicolor (millet), Zea mays (maize), Saccharum officinarum (sugar cane) and Oryza sative (rice).

Consequently in one embodiment the invention relates to a monocotyledonous organism comprising a nucleic acid sequence according to the invention, which comprises a mutation which causes a reduction in the activity of a protein encoded by the nucleic acid molecules according to the invention in the organisms or parts thereof.

A further object of the invention relates to transgenic plants, transformed with at least
a) one nucleic acid sequence, which comprises nucleic acid molecules according to SEQ. ID No: 3, 5, 7, 9, 12, 14, 16, 22, 24, 26, 28, 30, or 32, comprise, the nucleic acid sequences complementary thereto, and the nucleic acid molecules coding for functional equivalents of the polypeptides according to SEQ. ID No.: 2, 4, 6, 8, 10, 11, 13, 15, 17, 23, 25, 27, 29, 31 or 33, which preferably do not correspond to the SEQ ID No: 1, 18, 20 and 34,
b) one double-stranded RNA nucleic acid molecule (dsRNA molecule), which causes a the decrease in a callose synthase, where the sense strand of said dsRNA molecule displays at least a homology of 30%, preferably at least 40%, 50%, 60%, 70% or 80%, particularly preferably at least 90%, very particularly preferably 100% to a nucleic acid molecule according to SEQ. ID No: 3, 5, 7, 9, 12, 14, 16, 22, 24, 26, 28, 30, or 32, or comprises a fragment of at least 17 base pairs, preferably at least 18, 19, 20, 21, 22, 23, 24, 25, 26, 27, 28, 29 or 30 base pairs, particularly preferably at least 40, 50, 60, 70, 80 or 90 base pairs, very particularly preferably at least 100, 200, 300 or 400 base pairs, most preferably of all at least 500, 600, 700, 800, 900 or more base pairs, which displays at least a 50%, 60%, 70% or 80%, particularly preferably at least 90%, very particularly preferably 100% homology to a nucleic acid molecule according to SEQ. ID No: 1, 3, 5, 7, 9, 12, 14, 16, 22, 24, 26, 28, 30, or 32, but preferably do not correspond to the SEQ ID No: 1, 18, 20 and 34
c) one transgenic expression cassette, which includes one of the nucleic acid sequences according to the invention, or a vector according to the invention, and cells, cell cultures, tissue, parts, such as for example in plant organisms leaves, roots, etc. or reproductive material derived from such organisms.

Host or starting organisms preferred as transgenic organisms are in particular plants according to the definition stated above. For example all genera and species of higher and lower plants which belong to the Liliopsidae class. In one embodiment, the transgenic organism is a mature plant, seed, shoot and embryo, and parts, reproductive material and cultures, for example cell cultures, derived therefrom. “Mature plant” means plants at any development stage beyond the embryo. “Embryo” means a young, immature plant at an early development stage. Plants particularly preferable as host organisms are plants to which the process according to the invention for the attainment of a pathogen resistance according to the criteria stated above can be applied. In one embodiment, the plant is a monocotyle plant such as for example wheat, oats, millet, barley, rye, maize, rice, buckwheat, Sorghum, Triticale, spelt or sugar cane, in particular selected from the species Hordeum vulgare (barley), Triticum aestivum (wheat), Triticum aestivum subsp. spelta (spelt), Triticale, Avena sative (oats), Secale cereale (rye), Sorghum bicolor (millet), Zea mays (maize), Saccharum officinarum (sugar cane) or Oryza sative (rice).

The production of the transgenic organisms can be effected with the process described above for the transformation or transfection of organisms.

A further object of the invention relates to the transgenic plants described according to the invention which in addition have an increased Bax inhibitor 1 activity, wherein plants which display an increased Bax inhibitor 1 activity in mesophyllic cells or root cells are preferable, transgenic plants which belong to the Poaceae family and display an increased Bax inhibitor 1 activity in mesophyllic cells or root cells are particularly preferable, transgenic plants selected from the plant genera Hordeum, Avena, Secale, Triticum, Sorghum, Zea, Saccharum and Oryza are most preferable, and the plant species Hordeum vulgare (barley), Triticum aestivum (wheat), Triticum aestivum subsp. spelta (spelt), Triticale, Avena sative (oats), Secale cereale (rye), Sorghum bicolor (millet), Zea mays (maize), Saccharum officinarum (sugar cane) and Oryza sative (rice) are most preferable of all.

A further object of the invention relates to the use of the transgenic organisms according to the invention and the cells, cell cultures and parts derived therefrom, such as for example in transgenic plant organisms, roots, leaves etc., and transgenic reproductive material such as seeds or fruit, for the production of foodstuffs or forage, pharmaceuticals or fine chemicals.

In one embodiment, the invention in addition relates to a process for the recombinant production of pharmaceuticals or fine chemicals in host organisms, wherein a host organism or a part thereof is transformed with one of the nucleic acid molecules expression cassettes described above and this expression cassette comprises one or several structural genes which code for the desired fine chemical or catalyze the biosynthesis of the desired fine chemical, the transformed host organism is cultured and the desired fine chemical is isolated from the culture medium. This process is widely applicable for fine chemicals such as enzymes, vitamins, amino acids, sugars, fatty acids, natural and synthetic flavorings, perfumes and colorants. Particularly preferable is the production of tocopherols and tocotrienols and carotenoids. The culturing of the transformed host organisms and the isolation from the host organisms or from the culture medium is effected by processes well known to the skilled person. The production of pharmaceuticals, such as for example antibodies or vaccines is described in Hood E E, Jilka J M (1999). Curr Opin Biotechnol. 10(4):382-6; Ma J K, Vine N D (1999). Curr Top Microbiol Immunol. 236:275-92.

According to the invention, the expression of a structural gene can naturally also be effected or influenced independently of the performance of the process according to the invention or the use of the objects according to the invention.

### Sequences

| | |
|---|---|
| 1. SEQ ID No: 1 | nucleic acid sequence coding for |
| | the callose synthase polypep- |
| | tide-1 (HvCSL-1) from Hordeum |
| | vulgare. |
| 2. SEQ ID No: 2 | amino acid sequence of the |
| | callose synthase polypeptide-1 |
| | from Hordeum vulgare. |
| 3. SEQ ID No: 3 | nucleic acid sequence coding for |
| | the callose synthase polypep- |
| | tide-2 (HvCSL-2) from Hordeum |
| | vulgare. |
| 4. SEQ ID No: 4 | amino acid sequence of the |
| | callose synthase polypeptide-2 |
| | from Hordeum vulgare. |
| 5. SEQ ID No: 5 | nucleic acid sequence coding for |
| | the callose synthase polypep- |
| | tide-3 (HvCSL-3) from Hordeum |
| | vulgare. |
| 6. SEQ ID No: 6 | amino acid sequence of the |
| | callose synthase polypeptide-3 |
| | from Hordeum vulgare. |
| 7. SEQ ID No: 7 | nucleic acid sequence coding for |
| | the callose synthase polypep- |
| | tide-7 (HvCSL-7) from Hordeum |
| | vulgare. |
| 8. SEQ ID No: 8 | amino acid sequence of the |
| | callose synthase polypeptide-7 |
| | from Hordeum vulgare. |
| 9. SEQ ID No: 9 | nucleic acid sequence coding for |
| | the callose synthase polypep- |
| | tide-1 (ZmCSL-1) from Zea mays. |
| 10. SEQ ID No: 10 | amino acid sequence of the |
| | callose synthase polypeptide-1 |
| | (reading frame +1) from maize |
| | (Zea mays). |
| 11. SEQ ID No: 11 | amino acid sequence of the |
| | callose synthase polypeptide-1 |
| | (reading frame +2) from maize |
| | (Zea mays). |
| 12. SEQ ID No: 12 | nucleic acid sequence coding for |
| | the callose synthase polypep- |
| | tide-1a (ZmCSL-1a) from Zea |
| | mays. |
| 13. SEQ ID No: 13 | amino acid sequence of the |
| | callose synthase polypeptide-1a |
| | from maize (Zea mays). |
| 14. SEQ ID No: 14 | nucleic acid sequence coding for |
| | the callose synthase polypep- |
| | tide-2 (ZmCSL-2) from Zea mays. |
| 15. SEQ ID No: 15 | amino acid sequence of the |
| | callose synthase polypeptide-2 |
| | from Zea mays. |
| 16. SEQ ID No: 16 | nucleic acid sequence coding for |
| | the callose synthase polypep- |
| | tide-3 (ZmCSL-3) from Zea mays. |
| 17. SEQ ID No: 17 | amino acid sequence of the |
| | callose synthase polypeptide-3 |
| | from Zea mays. |
| 18. SEQ ID No: 18 | nucleic acid sequence coding for |
| | the callose synthase polypep- |
| | tide-1 (OsCSL-1) from Oryza |
| | sativa. |
| 19. SEQ ID No: 19 | amino acid sequence of the |
| | callose synthase polypeptide-1 |
| | from Oryza sativa. |
| 20. SEQ ID No: 20 | nucleic acid sequence coding for |
| | the callose synthase polypep- |
| | tide-2 (OsCSL-2) from Oryza |
| | sativa. |
| 21. SEQ ID No: 21 | amino acid sequence of the |
| | callose synthase polypeptide-2 |
| | from Oryza sative. |
| 22. SEQ ID No: 22 | nucleic acid sequence coding for |
| | the callose synthase polypep- |
| | tide-1 from (TaCSL-1) Triticum |
| | aestivum. |
| 23. SEQ ID No: 23 | amino acid sequence of the |
| | callose synthase polypeptide-1 |
| | from Triticum aestivum. |
| 24. SEQ ID No: 24 | nucleic acid sequence coding for |
| | the callose synthase polypep- |
| | tide-2 (TaCSL-2) from Triticum |
| | aestivum. |
| 25. SEQ ID No: 25 | amino acid sequence of the |
| | callose synthase polypeptide-2 |
| | from Triticum aestivum. |
| 26. SEQ ID No: 26 | nucleic acid sequence coding for |
| | the callose synthase polypep- |
| | tide-4 (TaCSL-4) from Triticum |
| | aestivum. |
| 27. SEQ ID No: 27 | amino acid sequence of the |
| | callose synthase polypeptide-4 |
| | from Triticum aestivum. |
| 28. SEQ ID No: 28 | nucleic acid sequence coding for |
| | the callose synthase polypep- |
| | tide-5 (TaCSL-5) from Triticum |
| | aestivum. |
| 29. SEQ ID No: 29 | amino acid sequence of the |
| | callose synthase polypeptide-5 |
| | from Triticum aestivum. |
| 30. SEQ ID No: 30 | nucleic acid sequence coding for |
| | the callose synthase polypep- |
| | tide-6 (TaCSL-6) from Triticum |
| | aestivum. |
| 31. SEQ ID No: 31 | amino acid sequence of the |
| | callose synthase polypeptide-6 |
| | from Triticum aestivum. |
| 32. SEQ ID No: 32 | nucleic acid sequence coding for |
| | the callose synthase polypep- |
| | tide-7 (TaCSL-7) from Triticum |
| | aestivum. |
| 33. SEQ ID No: 33 | amino acid sequence of the |
| | callose synthase polypeptide-7 |
| | from Triticum aestivum. |
| 34. SEQ ID No: 34 | nucleic acid sequence coding for |
| | the glucan synthase-like poly- |
| | peptide-5 from A. thalina |
| | (accession No. NM_116593). |
| 35. SEQ ID No:.35 | amino acid sequence of the |
| | callose synthase coding for the |
| | glucan synthase-like polypep- |
| | tide-5 from A. thalina. |
| 36. SEQ ID No: 36 | nucleic acid sequence coding for |
| | the Bax inhibitor 1 from Hordeum |
| | vulgare. GenBank Acc.-No.: |
| | AJ290421 |
| 37. SEQ ID No: 37 | amino acid sequence of the Bax |
| | inhibitor 1 polypeptide from |
| | Hordeum vulgare. |
| 38. SEQ ID No: 38 | nucleic acid sequence coding for |
| | the Bax inhibitor 1 from |
| | Nicotiana tabacum. (GenBank |
| | Acc.-No.: AF390556) |
| 39. SEQ ID No: 39 | amino acid sequence of the Bax |
| | inhibitor 1 polypeptide from |
| | Nicotiana tabacum. |
| 40. SEQ ID No: 40 | Hei131 |
| | 5′-GTTCGCCGTTTCCTCCCGCAACT-3′ |
| 41. SEQ ID No: 41 | Gene Racer 5′-Nested primer, |
| | Invitrogen |
| | 5′-GGACACTGACATGGACTGAAGGAGTA-3′ |
| 42. SEQ ID No: 42 | RACE-HvCSL1: |
| | 5′-GCCCAACATCTCTTCCTTTACCAACC |
| | T-3′ |
| 43. SEQ ID No: 43 | GeneRacer™ 5′ primer: |
| | 5′-CGACTGGAGCACGAGGACACTGA-3 |
| 44. SEQ ID No: 44 | RACE-5′nested HvCSL1: |
| | 5′-TCTGGCTTTATCTGGTGTTGGAGAAT |
| | C-3′ |
| 45. SEQ ID No: 45 | GeneRacer™ 3′ primer: |
| | 5′-GCTGTCAACGATACGCTACGTAACG-3 |
| 46. SEQ ID No: 46 | GeneRacer™ 3′-Nested primer: |
| | 5′-CGCTACGTAACGGCATGACAGTG-3 |
| 47. SEQ ID No: 47 | M13-fwd: |
| | 5′-GTAAAACGACGGCCAGTG-3′ |
| 48. SEQ ID No: 48 | M13-Rev: |
| | 5′-GGAAACAGCTATGACCATG-3′ |
| 49. SEQ ID No: 49 | Hei 97 forward |
| | 5′-TTGGGCTTAATCAGATCGCACTA-3′ |
| 50. SEQ ID No: 50 | Hei 98 reverse |
| | 5′-GTCAAAAAGTTGCCCAAGTCTGT-3′ |

### EXAMPLES

### General Methods

The chemical synthesis of oligonucleotides can for example be effected, in known manner, by the phosphoamidite method (Voet, Voet, 2^{nd} Edn., Wiley Press New York, pp. 896-897). The cloning steps performed in the context of the present invention, such as for example restriction cleavage, agarose gel electrophoresis, purification of DNA fragments, transfer of nucleic acids onto nitrocellulose and nylon membranes, linking of DNA fragments, transformation of E. coli cells, culturing of bacteria, growth of phages and sequence analysis of recombinant DNA are performed as described in Sambrook et al. (1989) Cold Spring Harbor Laboratory Press; ISBN 0-87969-309-6. The sequencing of recombinant DNA molecules is effected with a laser fluorescence DNA sequencer from the firm MWG-Licor by the method of Sanger (Sanger et al. (1977) Proc Natl Acad Sci USA 74:5463-5467).

### Example 1

### Plants, Pathogens and Inoculation

The barley variety Ingrid comes from Patrick Schweizer, Institute for Plant Genetics and Crop Plant Research, Gatersleben. The variety Pallas and the back-crossed line BCIngrid-mlo5 was provided by Lisa Munk, Department of Plant Pathology, Royal Veterinary and Agricultural University, Copenhagen, Denmark. Its preparation has been described (Kølster P et al. (1986) Crop Sci 26: 903-907).

The seed, pregerminated for 12 to 36 hrs in the dark on moist filter paper, is, unless otherwise described, laid out, 5 grains at the edge of each square pot (8×8 cm) in Fruhstorf earth of type P, covered with earth and regularly watered with tap water. All plants are cultivated in air-conditioned cabinets or chambers at 16 to 18° C., 50 to 60% relative atmospheric humidity and 16 hour light/8 hour darkness cycle at 3000 and 5000 lux (50 and 60 μmols-¹m-² photon flux density) for 5 to 8 days, and used in the experiments at the embryo stage. In experiments in which applications on primary leaves are performed, these are completely developed.

Before transient transfection experiments are performed, the plants are cultivated in air-conditioned cabinets or chambers at 24° C. in daytime, and 20° C. by night, 50 to 60% relative atmospheric humidity and a 16 hour light/8 hour darkness cycle at 30 000 lux.

For the inoculation of barley plants, powdery barley mildew Blumeria graminis (DC) Speer f.sp. hordei Em. Marchal of the A6 strain (Wiberg A (1974) Hereditas 77: 89-148) (BghA6) is used. This was provided by the Institute for Biometry, JLU Gieβen. The further growth of the inoculum is effected in air-conditioned chambers under the same conditions as described above for the plants, by transfer of the conidia from infected plant material onto regularly grown, 7-day old barley plants cv. Golden Promise at a density of 100 conidia/mm².

The inoculation with BghA6 is effected using 7-day old embryos by shaking off the conidia of already infected plants in an inoculation tower with approx. 100 conidien/mm² (unless otherwise stated).

### Example 2

### RNA Extraction

Total RNA is extracted from 8 to 10 primary leaf segments (length 5 cm) with “RNA Extraction Buffer” (AGS, Heidelberg, Germany).

For this, central primary leaf segments of 5 cm length are harvested and homogenized in liquid nitrogen in mortars. The homogenizate is stored at −70° C. until RNA extraction.

Total RNA is extracted from the deep-frozen leaf material with the aid of an RNA extraction kit (AGS, Heidelberg). For this, 200 mg of the deep-frozen leaf material in a microcentrifuge tube (2 mL) is covered with a layer of 1.7 mL of RNA extraction buffer (AGS) and immediately thoroughly mixed. After addition of 200 μL of chloroform, it is again mixed well and shaken for 45 mins at room temperature on a horizontal shaker at 200 rpm. Next, it is centrifuged for 15 min at 20 000 g and 4° C. for phase separation, the upper aqueous phase is transferred into a new microcentrifuge tube and the lower one discarded. The aqueous phase is again cleaned with 900 μL of chloroform, by 3 times homogenizing for 10 secs and again centrifuging (see above) and removing. For the precipitation of the RNA, 850 μL of 2-propanol are then added, and the mixture is homogenized and placed on ice for 30 to 60 mins. After this, it is centrifuged for 20 mins (see above), the supernatant is carefully decanted off, 2 mL of 70% ethanol (−20° C.) are pipetted into this, mixed and again centrifuged for 10 mins. The supernatant is then again decanted off and the pellet carefully freed from liquid residues using a pipette before it is dried at a clean air workstation in a clean air flow. After this, the RNA is dissolved in 50 μL of DEPC water on ice, thoroughly mixed and centrifuged for 5 min (see above). 40 μl of the supernatant are transferred into a new microcentrifuge tube as RNA solution and stored at −70° C.

The concentration of the RNA is determined photometrically. For this, the RNA solution is diluted 1:99 (v/v) with distilled water and the extinction (Photometer DU 7400, Beckman) measured at 260 nm (E_{260 nm}=1 at 40 ∝g RNA/mL). On the basis of the calculated RNA contents, the concentrations of the RNA solutions are then adjusted with DEPC water to 1 μg/μL and checked in the agarose gel.

For the checking of the RNA concentrations in the horizontal agarose gel (1% agarose in 1×MOPS buffer with 0.2 μg/mL ethidium bromide), 1 μL of RNA solution is treated with 1 μL of 10×MOPS, 1 μL of dye marker and 7 μL of DEPC water, separated by size at 120 V voltage in the gel in 1×MOPS run buffer for 1.5 hrs and photographed under UV light. Any concentration differences in the RNA extracts are adjusted with DEPC water and the adjustment again checked in the gel.

### Example 3

### Cloning of the HvCSL1 cDNA Sequence from Barley

The cDNA fragments needed for the isolation of the HvCSL1 cDNA, and its cloning, sequencing and the preparation of probes were obtained by RT-PCR using the “One Step RT-PCR Kit” (Life Technologies, Karlsruhe, Germany or Qiagen, Hilden, Germany). For this, total RNA from barley seedlings was used as the template. The RNA was isolated from cv. Ingrid 7 days after germination. In addition, RNA from cv. Ingrid and the back-crossed lines with mlo5 was isolated 1, 2 and 5 days after inoculation with BghA6 on the 7^{th} day after germination. For the RT-PCR, the following primers were used:

Hei131

(SEQ ID No:40)

5′-GTTCGCCGTTTCCTCCCGCAACT-3′
and
Gene Racer 5′-Nested primer, Invitrogen

(SEQ ID No:41)

5′-GGACACTGACATGGACTGAAGGAGTA-3′

For each reaction (25 μL mixture), 1000 ng of total RNA, 0.4 mM dNTPs, 0.6 mM each of OPN-1 and OPN-2 primers, 10 μl of RNase inhibitor and 1 μl of enzyme mix in 1×RT buffer (one step RT-PCR Kit, Qiagen, Hilden) were used.

The following temperature program is used (PTC-100TM Model 96V; MJ Research, Inc., Watertown, Mass.):

1 cycle with 30 mins at 50° C.
1 cycle with 150 secs at 94° C.
30 cycles with 94° C. for 45 sec, 55° C. for 1 min and 72° C. for 2 min
1 cycle with 72° C. for 7 min

The PCR product was separated by 2% w/v agarose gel electrophoresis. An RT-PCR product with a total of 249 bp was obtained. The corresponding cDNA was isolated from an agarose gel and cloned into the pTOPO vector (Invitrogen Life Technologies Co.) by T-overhang ligation. The cDNAs were sequenced from the plasmid-DNA using the “Thermo Sequenase Fluorescent Labeled Primer Cycle Sequencing Kit” (Amersham, Freiburg, Germany).

The cDNA sequence of HvCSL1 was extended by means of the RACE technology using the “GeneRacer Kit” (INVITROGENE Life Technologies). For this, 100 ng of poly-A mRNA, 1 μL of 10×CIP buffer, 10 units of RNAse inhibitor, 10 units of CIP (“calf intestinal phosphatase”) and DEPC-treated water to a total volume of 10 μL were processed for 1 hr at 50° C. For the precipitation of the RNA, a further 90 μL of DEPC water and 100 μL of phenol:chloroform were added and intensively mixed for approx. 30 secs. After 5 mins centrifugation at 20 000 g, the upper phase was treated with 2 μl of 10 mg/ml mussel glycogen and 10 μl of 3 M sodium acetate (pH 5.2) in a new microreaction vessel. 220 μl of 95% ethanol were added and the mixture incubated on ice. Next, the RNA was precipitated by centrifugation for 20 mins at 20 000 g and 4° C. The supernatant was discarded, 500 μl of 75% ethanol were added, briefly vortexed and again centrifuged for 2 mins (20 000 g). The supernatant was again discarded, the precipitate dried for 2 mins at room temperature in the air and then suspended in 6 μl of DEPC water. mRNA CAP structures were removed by addition of 1 μl of 1×TAP buffer, 10 units of RNAsin and 1 unit of TAP (“tobacco acid pyrophosphatase”). The mixture was incubated for 1 hr at 37° C. and then cooled on ice. The RNA was again precipitated, as described above, and transferred into a reaction vessel with 0.25 μg of GeneRacer oligonucleotide primer. The oligonucleotide primer was resuspended in the RNA solution, the mixture incubated for 5 mins at 70° C. and then cooled on ice. 1 μl of 10× ligase buffer, 10 mM ATP, 1 unit of RNAsin and 5 units of T4 RNA ligase were added and the reaction mixture incubated for 1 hr at 37° C. The RNA was again precipitated, as described above, and resuspended in 13 μl of DEPC water. 10 pMol of oligo-dT primer were added to the RNA, heated immediately to 70° C. and again cooled on ice. 1 μL of each dNTP solution (25 mM), 2 μL of 10×RT buffer, 5u (1 μl) of AMV reverse transcriptase and 20 units of RNAsin were added and the reaction solution incubated for 1 hr at 42° C. and then for 15 mins at 85° C. The primary strand cDNA thus prepared was stored at −20° C.

For the amplification of the 5′ and 3′-cDNA ends, the following primers were used:

RACE-HvCSL1:

(SEQ ID No:42)

5′-GCCCAACATCTCTTCCTTTACCAACCT-3′
GeneRacer™ 5′ primer:

(SEQ ID No:43)

5′-CGACTGGAGCACGAGGACACTGA-3
GeneRacer™ 5′-Nested primer:

(SEQ ID No:41)

5′-GGACACTGACATGGACTGAAGGAGTA-3
RACE-HvCSL1-nested:

(SEQ ID No:44)

5′-TCTGGCTTTATCTGGTGTTGGAGAATC-3′
GeneRacer™ 3′ primer:

(SEQ ID No:45)

5′-GCTGTCAACGATACGCTACGTAACG-3
GeneRacer™ 3′-Nested primer:

(SEQ ID No:46)

5′-CGCTACGTAACGGCATGACAGTG-3

The mixture (total volume 25 μL) had the following composition:

| | |
|---|---|
| 1 | μl primer RACE-HvCSL1 (5 pmol/μL), |
| 0.5 | μl GeneRacer 5′ primer (10 pmol/μL) |
| 2.5 | μl 10× buffer Qiagen, |
| 2.5 | μl dNTPs (2 mM) |
| 0.5 | μl cDNA |
| 0.2 | μl QiagenTAG (5 u/microL) |
| 17.8 | μl H2O |

The PCR conditions were:

### 94° C. 5 min denaturation

5 cycles with
70° C. 30 secs (annealing),
72° C. 1 mins (extension),
94° C. 30 secs (denaturation)
   5 cycles with
68° C. 30 secs (annealing),
72° C. 1 mins (extension),
94° C. 30 secs (denaturation)
   28 cycles with
66° C. 30 secs (annealing),
72° C. 1 mins (extension),
94° C. 30 secs (denaturation)
72° C. 10 mins concluding extension
4° C. cooling until further processing

The PCR yielded a product of approx. 400 bp product. Starting with this, a “nested” PCR was performed with the HvCSL1-specific oligonucleotide primer and the “GeneRacer Nested 5′ primer”:
94° C. 5 mins denaturation
   30 cycles with
64° C. 30 secs (annealing),
72° C. 1 min (extension),
94° C. 30 secs (denaturation)
72° C. 10 mins concluding extension
4° C. cooling until further processing

The PCR product obtained was isolated via a gel, extracted from the gel and cloned in pTOPO by T-overhang ligation and sequenced. The sequence quoted under SEQ ID No: 1 is thus identical with the HvCSL1 sequence from barley.

### Example 4

### Quantitative Polymerase Chain Reaction (Q-PCR)

7 days after germination, leaf material from barley cv. Ingrid was with conidiospores of the avirulent powdery mildew fungus Blumeria graminis f. sp. tritici and of the virulent powdery mildew fungus Blumeria graminis f. sp. hordei. 0, 24 and 48 hrs after inoculation, leaf material from these interactions was harvested. In addition, non-infected material was harvested as a control at the same time points.

The harvested leaf material was packed in aluminum foil and immediately deep frozen in liquid N₂. It was stored at −80° C. After grinding of the leaf material, was the RNA was isolated with the RNeasy Maxi Kite from the QIAGEN Co. (Hilden) in accordance with the manufacturer's instructions. The elution was effected with 1.2 ml of RNase-free water. Next the RNA was precipitated and taken up in the appropriate volume of H₂O. The RNA concentrations were determined with the Eppendorf BioPhotometer 6131.

TABLE 1
Concentrations of the barley total RNA

| Sample | Concentration in μg/ml |
|---|---|
| Ingrid 0 hrs | 2.2 |
| Ingrid 24 hrs | 2.9 |
| Ingrid 48 hrs | 3.0 |
| Ingrid Bgt 0 hrs | 2.4 |
| Ingrid Bgt 24 hrs | 3.6 |
| Ingrid Bgt 48 hrs | 3.6 |
| Ingrid Bgh 0 hrs | 2.2 |
| Ingrid Bgh 24 hrs | 3.0 |
| Ingrid Bgh 48 hrs | 1.4 |

For the quantitative PCR, the RNA samples from Table 1 were used. Any DNA still present was first digested from the individual RNA samples. The digestion was set up as follows with DNA-free™ from the AMBION Co. (Huntingdon, USA):

| | | |
|---|---|---|
| Total volume | 60 | μl |
| RNA | 50 | μl |
| 10× DNase I buffer | 6 | μl |
| DNase I (2 U/μl) | 1 | μl |
| H₂O q.s.p. 60 μl | 3 | μl |

The mixture was incubated for 60 mins at 37° C. Next, 6 μl of DNase inactivation reagent were added and the preparation well mixed. After a further incubation time of 2 mins at room temperature, the solution was centrifuged at 10 000 g for 1 min, in order to pelletize the DNase inactivation reagent. The RNA was transferred into a new vessel and kept at −20° C.

After the digestion, the RNA was transcribed into DNA. Departing from the manufacturer's instructions, the preparation was made up with the Taq Man Reverse Transcription Reagents from the APPLIED BIOSYSTEMS Co. (Applera Deutschland GmbH, Darmstadt, Germany):

| | |
|---|---|
| Total volume | 20 μl |
| RNA | 3 μl |
| 25 mM MgCl₂ | 4.4 μl |
| dNTP-Mix (10 mM) | 4 μl |
| 50 μM random hexamer | 1 μl |
| 10× RT buffer | 2 μl |
| Rnase inhibitor | 0.4 μl |
| Multiscribe RT (50 U/μl) | 1.5 μl |
| H₂O nuclease-free | 3.7 μl |

The mixture was incubated for 10 mins at 25° C., followed by an incubation at 37° C. for 60 mins. Finally, the mixture was heat-inactivated for 5 mins at 95° C.

3 μl of the transcribed DNA was used for each quantitative PCR. As the internal standard, 18S rRNA was determined simultaneously. A triple determination was carried out on all samples. The mixtures were pipetted onto a 96-well plate. First the SYBR Green® Master Mix was taken with the primers and the appropriate quantity of water, then the DNA were individually pipetted into this and the preparation mixed.

| | |
|---|---|
| Total volume | 25 μl |
| cDNA | 3 μl |
| 2× SYBR Green ® Master Mix | 12.5 μl |
| Forward primer, 200 nM | x μl |
| Reverse primer, 200 nM | x μl |
| H₂O nuclease-free q.s.p. 25 μl | x μl |

TABLE 2
QPCR primer for barley

| | Volume | | |
|---|---|---|---|
| Barley | per prep. | | |
| primer | in ul | Product | Sequence |
| Hei 97 | 0.05 | HvCSL1 | TTGGGCTTAATCAGATCGCACTA |
| forward | | | |
| Hei 98 | 0.05 | HvCSL1 | GTCAAAAAGTTGCCCAAGTCTGT |
| reverse | | | |

The primers were searched out from the EST sequence using the program Primer Express from APPLIED BIOSYSTEMS (Applera Deutschland GmbH, Darmstadt, Germany).

The plate was centrifuged at RT and 2500 rpm (centrifuge 4K15C, SIGMA, Osterode, Germany) for 1 min, then the samples were estimated directly. For the quantitative PCR, the ABI PRISM 7000 instrument from the APPLIED BIOSYSTEMS Co. (Applera Deutschland GmbH, Darmstadt, Germany) was used. The assessment was performed using the program ABI PRISM 7000 SDS from the APPLIED BIOSYSTEMS Co. (Applera Deutschland GmbH, Darmstadt, Germany).

In Table 3, the expression data from HvCSL1 are shown. The measurement was carried out twice, and a threefold determination of the individual measurement values was made. The averaged values are shown in each case, and the corresponding standard deviation.

TABLE 3
Expression data from HvCSL1

| Sample | Plant | Gene | Standard | |
|---|---|---|---|---|
| number | material | expression | Deviation | Calibrator |
| 1 | Ingrid cont. 0 hr | 1.01 | 0.11 | Ingrid |
| 2 | Ingrid cont. 24 hrs | 0.5 | 0.04 | 0 hr control |
| 3 | Ingrid cont. 48 hrs | 2.5 | 0.10 | |
| 4 | Ingrid + Bgt 0 hpi | 1.00 | 0.06 | Ingrid + Bgt |
| 5 | Ingrid + Bgt 24 hpi | 1.01 | 0.23 | 0 hpi |
| 6 | Ingrid + Bgt 48 hpi | 0.75 | 0.06 | |
| 7 | Ingrid + Bgh 0 hpi | 1.00 | 0.08 | Ingrid + Bgh |
| 8 | Ingrid + Bgh 24 hpi | 9.75 | 0.03 | 0 hpi |
| 9 | Ingrid + Bgh 48 hpi | 7.25 | 0.14 | |

The expression data from HvGsl1 are shown, which are made up of two measurements with a 3-fold determination in each case. The RNA used was DNA-digested and then transcribed into DNA with the Taq Man Reverse Transcription Reagents. 18S rRNA was used as the endogenous control in the measurement. The 0 hrs value of the measurement was used as the comparison value or calibrator for each interaction.

The data show that, consistently with the role of HvCSL1 as a compatibility factor, the expression in the compatible interaction with Blumeria graminis f. sp. hordei is markedly increased compared to the incompatible interaction with Blumeria graminis f. sp. tritici.

### Example 5

### Northern-Blot Analysis

In preparation for the Northern Blotting, the RNA is separated in agarose gel under denaturing conditions. For this, a portion of RNA solution (corresponding to 5 μg RNA) is mixed with an equal volume of sample buffer (containing ethidium bromide), denatured for 5 mins at 94° C., placed on ice for 5 mins, briefly centrifuged and applied onto the gel. The 1×MOPS gel (1.5% Agarose, ultra pure) comprises 5 volume percent of concentrated formaldehyde solution (36.5% [v/v]). The RNA is separated for 2 hrs at 100 V and then blotted.

The Northern blotting is effected as an upward RNA transfer in the capillary flow. For this, the gel is first rocked for 30 mins in 25 mM sodium hydrogen/dihydrogen phosphate buffer (pH 6.5) and cut to shape. A Whatman paper is prepared so that it lay on a horizontal plate and projects on 2 sides into a bath containing 25 mM sodium hydrogen/dihydrogen phosphate buffer (pH 6.5). The gel is laid on this paper, uncovered parts of the Whatman paper being covered with a plastic film. The gel is then covered with a positively charged nylon membrane (Boehringer-Mannheim) with no air bubbles, after which the membrane is again covered with absorbent paper in several layers, to a height of about 5 cm. The absorbent paper is further weighted with a glass plate and a 100 g weight. The blotting takes place overnight at room temperature. The membrane is briefly rocked in doubly distilled water and is irradiated with UV light with a light energy of 125 mJ in the Crosslinker (Biorad) to fix the RNA. The checking of the uniform RNA transfer onto the membrane is performed on the UV light bench.

For the detection of barley mRNA, 10 μg of total RNA from each sample is separated on an agarose gel and blotted by capillary transfer onto a positively charged nylon membrane. The detection is performed with the DIG system.

Preparation of the Probes: for the Hybridization with the mRNAs to be Detected, RNA probes labeled with digogygenin or fluorescein are prepared. These are generated by in vitro transcription of a PCR product by means of a T7 or SP6 RNA polymerase with labeled UTPs. The plasmid vectors described above serve as the template for the PCR supported amplification.

Depending on the orientation of the insert, different RNA polymerases are used for the preparation of the antisense strand, T7 RNA polymerase or SP6 RNA polymerase.

The insert of the individual vector is amplified by PCR with flanking standard primers (M13 fwd and rev). Here the reaction runs with the following final concentrations in a total volume of 50 μL of PCR buffer (Silverstar):

M13-fwd:

(SEQ ID No:47)

5′-GTAAAACGACGGCCAGTG-3′
M13-Rev:

(SEQ ID No:48)

5′-GGAAACAGCTATGACCATG-3′

10% dimethyl sulfoxide (v/v)
2 ng/μL of each primer (M13 forward and reversed)
1.5 mM MgCl₂,
0.2 mM dNTPs,
4 units Taq polymerase (Silverstar),
2 ng/μL plasmid DNA.

The amplification takes place under temperature control in a Thermocycler (Perkin-Elmar 2400):

### 94° C. 3 mins denaturation

30 cycles with
94° C. 30 secs (denaturation)
58° C. 30 secs (annealing),
72° C. 1.2 mins (extension),
72° C. 5 mins concluding extension
4° C. cooling until further processing

The outcome of the reaction is checked in the 1% agarose gel. The products are then purified with a “High Pure PCR-Product Purification Kit” (Boehringer-Mannheim). About 40 μL of column eluate is obtained, which is checked again in the gel and stored at −20° C.

The RNA polymerization, the hybridization and the immunodetection are very largely performed according to the instructions of the manufacturer of the Kit for nonradio-active RNA detection (DIG System User's Guide, DIG-Luminescence detection Kit, Boehringer-Mannheim, Kogel et al. (1994) Plant Physiol 106:1264-1277). 4 μl of purified PCR product are treated with 2 μL of transcription buffer, 2 μl of NTP labeling mix, 2 μl of NTP mix and 10 μl of DEPC water. Next, 2 μL of the T7 RNA polymerase solution are pipetted in. The reaction is then performed for 2 hrs at 37° C. and then made up to 100 μL with DEPC water. The RNA probe is detected in the ethidium bromide gel and stored at −20° C.

In preparation for the hybridization, the membranes are first rocked for 1 hr at 68° C. in 2×SSC (salt, sodium citrate), 0.1% SDS buffer (sodium dodecylsulfate), the buffer being renewed 2 to 3 times. The membranes are then laid on the inner wall of hybridization tubes preheated to 68° C. and incubated for 30 mins with 10 mL of Dig-Easy hybridization buffer in the preheated hybridization oven. Meanwhile, 10 μL of probe solution are denatured in 80 μL of hybridization buffer at 94° C. for 5 mins, then placed on ice and briefly centrifuged. For the hybridization, the probe is then transferred into 10 mL of warm hybridization buffer at 68° C., and the buffer in the hybridization tubes replaced by this probe buffer. The hybridization is then likewise effected at 68° C. overnight.

Before immunodetection of RNA-RNA hybrids, the blots are stringently washed twice for 20 mins each time in 0.1% (w/v) SDS, 0.1×SSC at 68° C.

For the immunodetection, the blots are first rocked twice for 5 mins at RT in 2×SSC, 0.1% SDS. Next 2 stringent washing steps are carried out at 68° C. in 0.1×SSC, 0.1% SDS, each for 15 mins. The solution is then replaced by washing buffer without Tween. It is shaken for 1 min and the solution replaced by blocking reagent. After a further 30 mins' shaking, 10 μL of anti-fluorescein antibody solution are added and the mixture is shaken for a further 60 mins. This is followed by two 15-minute washing steps in washing buffer with Tween. The membrane is then equilibrated for 2 mins in substrate buffer and, after draining, is transferred onto a copying film. A mixture of 20 μL of CDP-Star™ and 2 mL of substrate buffer is then uniformly distributed on the “RNA side” of the membrane. Next, the membrane is covered with a second copying film and watertightly heat-sealed at the edges, with no air bubbles. The membrane is then covered with an X-ray film for 10 mins in a darkroom and this is then developed. The exposure time is varied depending on the strength of the luminescence reaction.

If not labeled extra, the solutions are contained in the range supplied in the Kit (DIG Luminescence Detection Kit, Boehringer-Mannheim). All others are prepared from the following stock solutions by dilution with autoclaved, distilled water. All stock solutions, unless otherwise specified, are made up with DEPC (such as DEPC water) and then autoclaved.
DEPC water: Distilled water is treated overnight at 37° C. with diethyl pyrocarbonate (DEPC, 0.1%, w/v) and then autoclaved
10×MOPS buffer: 0.2 M MOPS (morpholin-3-propanesulfonic acid), 0.05 M sodium acetate, 0.01 M EDTA, pH adjusted to pH 7.0 with 10 M NaOH
20×SSC (sodium chloride-sodium citrate, salt-sodium citrate): 3 M NaClo, 0.3 M trisodium citrate×2H₂O, pH adjusted to pH 7.0 with 4 M HCl.
1% SDS (sodium dodecylsulfate, sodium dodecylsulfate) sodium dodecylsulfate (w/v), without DEPC
RNA sample buffer: 760 ∝L formamide, 260 μL formaldehyde 100 μL ethidium bromide (10 mg/mL), 80 μL glycerol, 80 μL bromophenol blue (saturated), 160 μL 10×MOPS, 100 μL water.
10× washing buffer without Tween: 1.0 M maleic acid, 1.5 M NaCl; without DEPC, adjust to pH 7.5 with NaOH (solid, approx. 77 g) and 10 M NaOH.
Washing buffer with Tween: from washing buffer without Tween with Tween (0.3%, v/v)
10× blocking reagent: suspend 50 g of blocking powder (Boehringer-Mannheim) in 500 mL of washing buffer without Tween.
Substrate buffer: adjust 100 mM Tris (trishydroxymethylaminomethane), 150 mM NaCl to pH 9.5 with 4 M HCl.
10× dye marker: 50% glycerol (v/v), 1.0 mM EDTA pH 8.0, 0.25% bromophenol blue (w/v), 0.25% xylenecyanol (w/v).

### Example 6

### In Vitro Synthesis of HvCSL1-dsRNA

All plasmids which are used for the in vitro transcription contain the T7 and SP6 promoter (pGEM-T, Promega) at the respective ends of the inserted nucleic acid sequence, which enables the synthesis of sense and antisense RNA respectively. The plasmids can be linearized with suitable restriction enzymes, in order to ensure correct transcription of the inserted nucleic acid sequence and to prevent read-through in vector sequences.

For this, 10 μg of plasmid DNA is cleaved each time on the side of the insert located distally from the promoter. The cleaved plasmids are extracted into 200 μl of water with the same volume of phenol/chloroform/isoamyl alcohol, transferred into a new Eppendorf reaction vessel (RNAse-free) and centrifuged for 5 mins at 20 000 g. 180 μl of the plasmid solution are treated with 420 μl of ethanol, placed on ice and then precipitated by centrifugation for 30 mins at 20 000 g and −4° C. The precipitate is taken up in 10 μl of TE buffer.

For the preparation of the HvCSL1-dsRNA, the plasmid pTOPO-HvCSL1 is digested with SpeI and sense RNA transcribed with the T7 RNA polymerase. Further, pTOPO-HvCSL1 is digested with NcoI and antisense RNA transcribed with the SP6 RNA polymerase. RNA polymerases are obtained from Roche Molecular Biology, Mannheim, Germany.

Each transcription preparation contains, in a volume of 40 μl:

2 μl linearized plasmid DNA (1 μg)
2 μl NTP's (25 mM) (1.25 mM of each NTP)
4 μl 10× reaction buffer (Roche Molecular Biology),
1 μl RNAsin RNAsin (27 units; Roche Molecular Biology),
2 μl RNA polymerase (40 units)
29 μl DEPC water

After an incubation of 2 hrs at 37° C., one portion each of the reaction preparations from the transcription of the “sense” and “antisense” strand respectively are mixed, denatured for 5 mins at 95° C. and then hybridized with one another by cooling over 30 mins to a final temperature of 37° C. (“annealing”). Alternatively, after the denaturation, the mixture of sense and antisense strand can also be cooled for 30 mins at −20° C. The protein precipitate which is formed during denaturation and hybridization is removed by brief centrifugation at 20 800 g and the supernatant directly used for the coating of tungsten particles (see below). For the analysis, in each case 1 μl of each RNA strand and the dsRNA are separated on a non-denaturing agarose gel. A successful hybridization is revealed by a band shift to higher molecular weight compared to the single strands.

4 μl of the dsRNA are ethanol-precipitated (by addition of 6 μl of water, 1 μl of 3M sodium acetate solution and 25 μl of ethanol, and centrifugation for at least 5 mins at 20 000 g and 4° C.) and resuspended in 500 μl of water. The absorption spectrum between 230 and 300 nm is measured, or the absorption at 280 and 260 nm determined, in order to determine the purity and the concentration of the dsRNA. As a rule, 80 to 100 μg of dsRNA with an OD₂₆₀/OD₂₈₀ ratio of 1.80 to 1.95 are obtained. A digestion with DNase I can optionally be performed, but does not significantly affect subsequent results.

The dsRNA of the human thyroid receptor acts as the control dsRNA (starting vector pT7betaSaI (Norman C et al. (1988) Cell 55(6):989-1003); the sequence of the insert is described under the GenBank Acc.-No.: NM_{—}000461). For the preparation of the sense RNA, the plasmid is digested with PvuII, and for the antisense RNA with HindIII, and the RNA then transcribed with T7 or SP6 RNA polymerase respectively. The individual process steps for the preparation of the control dsRNA are performed analogously to those described above for the HvCSL1-dsRNA.

### Example 7

### Transient Transformation, RNAi and Evaluation of the Fungal Pathogen Development

Barley cv Ingrid leaf segments are transformed with a HvCSL1 dsRNA together with a GFP expression vector. Next, the leaves are inoculated with Bgh and the result analyzed after 48 hr by optical and fluorescence microscopy. The penetration in GFP-expressing cells is assessed by detection of haustoria in living cells and by assessment of the fungal development in precisely these cells. In all six experiments, the bombardment of barley cv Ingrid with HvCSL1 dsRNA resulted in a decreased number of cells successfully penetrated by Bgh compared to cells which were bombarded with a foreign control dsRNA (human thyroid hormone receptor dsRNA, TR). The resistance-inducing effect of the HvCSL1 dsRNA causes an average decrease in the efficiency of penetration by Bgh of at least 20%.

A process for the transient transformation was used which has already been described for the biolistic introduction of dsRNA into epidermal cells of barley leaves (Schweizer P et al. (1999) Mol Plant Microbe Interact 12:647-54; Schweizer P et al. (2000) Plant J 2000 24: 895-903). Tungsten particles with a diameter of 1.1 μm (particle density 25 mg/ml) are coated with dsRNA (preparation—see above) together with plasmid DNA of the vector pGFP (GFP under control of the pUBI promoter) as transformation marker. For this, the following quantities of dsRNA and reporter plasmid per shot are used for the coating: 1 μg of pGFP and 2 μg of dsRNA. Double-stranded RNA was synthesized in vitro by fusion of “sense” and “antisense” RNA (see above).

For microcarrier preparation, 55 mg of tungsten particles (M 17, diameter 1.1 μm; Bio-Rad, Munich, Germany) are washed twice with 1 ml of autoclaved distilled water and once with 1 mL of absolute ethanol, dried and taken up in 1 ml of 50% glycerine (approx. 50 mg/ml stock solution, Germany). The solution is diluted to 25 mg/ml with 50% glycerine, mixed well before use and suspended in the ultrasonic bath. For the microcarrier coating, per shot, 1 μg of plasmid, 2 μg of dsRNA (1 μL), 12.5 μl of tungsten particle suspension (25 mg/ml) and 12.5 μl of 1 M Ca(NO₃)₂ solution (pH 10) are added dropwise with constant mixing, allowed to stand for 10 mins at RT, briefly centrifuged and 20 μl removed from the supernatant. The residue with the tungsten particles is resuspended (ultrasonic bath) and used in the experiment.

Approx. 4 cm long segments of barley primary leaves are used. The tissues are laid on 0.5% Phytagar (GibcoBRL™ Life Technologies™, Karlsruhe) containing 20 μg/ml benzimidazole in Petri dishes (6.5 cm diameter) and directly before the particle shooting are covered at the edges with a template with a 2.2 cm×2.3 cm rectangular opening. The dishes are successively placed on the floor of the vacuum chamber (Schweizer P et al. (1999) Mol Plant Microbe Interact 12:647-54), over which a nylon net (mesh width 0.2 mm, Millipore, Eschborn, Germany) is inserted in as a diffuser on a perforated plate (5 cm over the floor, 11 cm below the macrocarrier, see below), in order to disperse particle clumps and slow the particle steam. For each shot, the macrocarrier installed at the top of the chamber (plastic sterile filter holder, 13 mm, Gelman Sciences, Swinney, UK) is loaded with 5.8 μL of DNA-coated tungsten particles (microcarrier, see below). The pressure in the chamber is reduced to 0.9 bar with a membrane vacuum pump (Vacuubrand, Wertheim, Germany) and the tungsten particles are shot onto the surface of the plant tissue with 9 bar helium gas pressure. Immediately after this, the chamber is ventilated. For the labeling of transformed cells, the leaves are shot with the plasmid (pGFP; Schweizer P et al. (1999) Mol Plant Microbe Interact 12:647-54; made available by Dr. P. Schweizer Schweizer P, Institute for Plant Genetics IPK, Gatersleben, Germany). Each time before the shooting of another plasmid, the macrocarrier is thoroughly cleaned with water. After four hours' incubation after the shooting, with slightly opened Petri dishes, RT and daylight, the leaves are inoculated with 100 conidia/mm² of the true barley mildew fungus (A6 strain) and incubated for a further 4036 to 48 hrs under the same conditions.

Leaf segments are bombarded with the coated particles using a “particle inflow gun”. 312 μg of tungsten particles are applied per shot. 4 hrs after the bombardment, inoculation with Blumeria graminis fsp. hordei mildew (A6 strain) is inoculated and assessed for signs of infection after a further 40 hrs. The result (e.g. the efficiency of penetration, defined as the percentage content of infected cells, which a with mature haustorium and a secondary hyphae (“secondary elongating hyphae”), is analyzed by fluorescence and optical microscopy. An inoculation with 100 conidia/mm² gives an infection frequency of approx. 50% of the transformed cells. For each individual experiment, a minimum number of 100 interaction sites are assessed. Transformed (GFP-expressing) cells are identified under excitation with blue light. Three different categories of transformed cells can be distinguished:
1. Penetrated cells which contain an easily recognizable haustorium. A cell with more than one haustorium is scored as one cell.
2. Cells which have been infected by a fungal appressorium, but contain no haustorium. A cell which is multiply infected by Bgh, but contains no haustorium, is scored as one cell.
3. Cells which have not been infected by Bgh.

Stomata cells and stomata subsidiary cells are excluded from the assessment. Surface structures of Bgh are analyzed by optical microscopy or fluorescence staining of the fungus with 0.1% Calcofluor (w/v in water) for 30 secs. The development of the fungus can easily be evaluated by fluorescence microscopy after staining with Calcofluor. In HvCSL1 dsRNA-transformed cells, the fungus does develop a primary and an appressorial germ tube, but no haustorium. Haustorium development is a precondition for the formation of a secondary hypha.

The relative penetration efficiency (RPE) is calculated as the difference between the penetration efficiency in transformed cells (transformation with HvCSL1 or control dsRNA) and the penetration efficiency in untransformed cells (average penetration efficiency 50-60%). The percentage RPE (% RPE) is calculated from the RPE minus 1 and multiplied by 100.

$$R P E = \frac{[ P E in HvCSL 1 dsRNA - transformed cells ]}{[ P E in control dsRNA - transformed cells ]}$$

$$\% R P E = 100 * ( \underline{R P E} - 1 )$$

The % RPE value (deviation from the average penetration efficiency of the control) serves for the determination of the susceptibility of cells which are transfected with HvCSL1 dsRNA.

With the control dsRNA, in five independent experiments, no difference as regards the penetration efficiency of Bgh was observed between the transfection with the control dsRNA and water.

1. A process for increasing the resistance against mesophyllic cell-penetrating pathogens in a plant, or an organ, tissue or a cell thereof, comprising reducing the callose synthase activity in a plant, or an organ, tissue or a cell thereof, wherein the callose synthase activity in the plant or an organ, tissue or a cell thereof is reduced in comparison to control plants.

2. The process according to claim 1, wherein the pathogens are selected from the Pucciniaceae, Mycosphaerellaceae and Hypocreaceae families.

3. The process according to claim 1, wherein the activity of a callose synthase protein comprising the sequences shown in SEQ ID NO: 2, 4, 6, 8, 10, 11, 13, 15, 17, 19, 21, 23, 25, 27, 29, 31, 33 or 35 or of a protein which displays a homology of at least 40% thereto is reduced.

4. The process according to claim 1, wherein the callose synthase activity available to the plant, the plant organ, tissue or the cell is reduced in that the activity of at least one polypeptide is reduced, which is encoded by a nucleic acid molecule comprising at least one nucleic acid molecule selected from the group consisting of:
a) a nucleic acid molecule which encodes a polypeptide comprising the sequence shown in SEQ ID NO:2, 4, 6, 8, 10, 11, 13, 15, 17, 19, 21, 23, 25, 27, 29, 31, 33 or 35;
b) a nucleic acid molecule which comprises at least one polynucleotide of the sequence shown in SEQ ID NO: 1, 3, 5, 7, 9, 12, 14, 16, 18, 20, 22, 24, 26, 28, 30, 32 or 34;
c) a nucleic acid molecule which encodes a polypeptide the sequence whereof displays an identity of at least 40% to the sequences SEQ ID NO: 2, 4, 6, 8, 10, 11, 13, 15, 17, 19, 21, 23, 25, 27, 29, 31, 33 or 35;
d) a nucleic acid molecule according to (a) to (c) which codes for a fragment or an epitope of the sequences according to SEQ. ID NO: 2, 4, 6, 8, 10, 11, 13, 15, 17, 19, 21, 23, 25, 27, 29, 31, 33 or 35;
e) a nucleic acid molecule which encodes a polypeptide which is recognized by a monoclonal antibody directed against a polypeptide which is encoded by the nucleic acid molecules according to (a) to (c); and
f) a nucleic acid molecule coding for a callose synthase, which hybridizes under stringent conditions with a nucleic acid molecule according to (a) to (c); and
g) a nucleic acid molecule coding for a callose synthase, which can be isolated from a DNA bank with the use of a nucleic acid molecule according to (a) to (c) or part fragments thereof of at least 15 nt, preferably 20 nt, 30 nt, 50 nt, 100 nt, 200 nt or 500 nt as a probe under stringent hybridization conditions;
or comprises a complementary sequence thereof.

5. The process according to claim 1 wherein
a) the expression of at least one callose synthase is reduced;
b) the stability of at least one callose synthase or of the mRNA molecules corresponding to this callose synthase is reduced;
c) the activity of at least one callose synthase is reduced;
d) the transcription at least one of the genes coding for a callose synthase is reduced by expression of an endogenous or artificial transcription factor; or
e) an exogenous factor reducing the callose synthase activity is added to the food or to the medium.

6. The process according to claim 4, wherein the decrease in the callose synthase activity is achieved by use of at least one process selected from the group consisting of:
a) the introduction of a nucleic acid molecule coding for ribonucleic acid molecules suitable for formation of double-stranded ribonucleic acid molecules (dsRNA), where the sense strand of the dsRNA molecule displays at least a homology of 30% to a nucleic acid molecule characterized in claim 4 or comprises a fragment of at least 17 base pairs, which displays at least a 50% homology to a nucleic acid molecule characterized in claim 4 (a) or (b),
b) the introduction of a nucleic acid molecule coding for an antisense ribonucleic acid molecule which displays at least a homology of 30% to the non-coding strand of a nucleic acid molecule characterized in claim 4 or comprises a fragment of at least 15 base pairs, which displays at least a 50% homology to a non-coding strand of a nucleic acid molecule characterized in claim 4 (a) or (b),
c) the introduction of a ribozyme which specifically cleaves the ribonucleic acid molecules encoded by one of the nucleic acid molecules mentioned in claim 4 or of an expression cassette ensuring the expression thereof,
d) the introduction of an antisense nucleic acid molecule as specified in (b), combined with a ribozyme or of an expression cassette ensuring the expression thereof,
e) the introduction of nucleic acid molecules coding for sense ribonucleic acid molecules coding for a polypeptide which is encoded by a nucleic acid molecule characterized in claim 4, in particular the proteins according to the sequences SEQ ID NO: 2, 4, 6, 8, 10, 11, 13, 15, 17, 19, 21, 23, 25, 27, 29, 31, 33 and/or 35 or for polypeptides which display at least a 40% homology to the amino acid sequence of a polypeptide which is encoded by the nucleic acid molecules named in claim 4,
f) the introduction of a nucleic acid molecule coding for a dominant-negative polypeptide suitable for the suppression of the callose synthase activity or of an expression cassette ensuring the expression thereof,
g) the introduction of a factor which can specifically bind the callose synthase polypeptide or the DNA or RNA molecules coding for this polypeptide or of an expression cassette ensuring the expression thereof,
h) the introduction of a viral nucleic acid molecule which causes a degradation of mRNA molecules coding for callose synthases or of an expression cassette ensuring the expression thereof,
i) the introduction of a nucleic acid construct suitable for the induction of a homologous recombination on genes coding for callose synthases; and
j) the introduction of one or more inactivating mutations into one or more genes coding for callose synthases.

7. The process according to claim 6, comprising
a) the introduction of a recombinant expression cassette comprising a nucleic acid sequence according to claim 6 (a-i) in functional linkage with a promoter active in plants, into a plant cell;
b) the regeneration of the plant from the plant cell, and
c) the expression of said nucleic acid sequence in a quantity and for a time sufficient to create or to increase a pathogen resistance in said plant.

8. The process according to claim 7, wherein the promoter active in plants is a pathogen-inducible promoter.

9. The process according to claim 7, wherein the promoter active in plants is a mesophyll-specific promoter.

10. The process according to claim 1, wherein a Bax inhibitor 1 protein is expressed in the plant, the plant organ, tissue or the cell.

11. The process according to claim 10, wherein the Bax inhibitor 1 is expressed under control of a mesophyll- and/or root-specific promoter.

12. The process according to claim 1, wherein the pathogen is selected from the species Puccinia triticina, Puccinia striiformis, Mycosphaerella graminicola, Stagonospora nodorum, Fusarium graminearum, Fusarium culmorum, Fusarium avenaceum, Fusarium poae or Microdochium nivale.

13. The process according to claim 1, wherein the plant is selected from the Poaceae plant family.

14. The process according to claim 1, wherein the plant is selected from the plant genera Hordeum, Avena, Secale, Triticum, Sorghum, Zea, Saccharum, or Oryza.

15. The process according to claim 1, wherein the plant is selected from the species Hordeum vulgare (barley), Triticum aestivum (wheat), Triticum aestivum subsp. spelta (spelt), Triticale, Avena sative (oats), Secale cereale (rye), Sorghum bicolor (millet), Saccharum officinarum (sugar cane), Zea mays (maize) and (maize), or Oryza sative (rice).

16. A nucleic acid molecule which encodes a polypeptide which comprises a polypeptide which is encoded by a nucleic acid molecule comprising a nucleic acid molecule selected from the group consisting of
a) a nucleic acid molecule which encodes a polypeptide comprising the sequence shown in SEQ ID NO: 4, 6, 8, 10, 11, 13, 15, 17, 23, 25, 27, 29, 31 or 33;
b) a nucleic acid molecule which comprises at least one polynucleotide of the sequence according to SEQ ID NO: 3, 5, 7, 9, 12, 14, 16, 22, 24, 26, 28, 30, or 32;
c) a nucleic acid molecule which encodes a polypeptide the sequence whereof displays an identity of at least 40% to the sequences SEQ ID NO: 4, 6, 8, 10, 11, 13, 15, 17, 23, 25, 27, 29, 31 or 33;
d) a nucleic acid molecule according to (a) to (c) which codes for a fragment or an epitope of the sequences according to SEQ. ID NO: 4, 6, 8, 10, 11, 13, 15, 17, 23, 25, 27, 29, 31 or 33;
e) a nucleic acid molecule which encodes a polypeptide which is recognized by a monoclonal antibody, directed against a polypeptide which is encoded by the nucleic acid molecules according to (a) to (c);
f) a nucleic acid molecule coding for a callose synthase which hybridizes under stringent conditions with a nucleic acid molecule according to (a) to (c); and
g) a nucleic acid molecule coding for a callose synthase, which can be isolated from a DNA bank with the use of a nucleic acid molecule according to (a) to (c) or part fragments thereof of at least 15 nt, preferably 20 nt, 30 nt, 50 nt, 100 nt, 200 nt or 500 nt as a probe under stringent hybridization conditions;
or comprises a complementary sequence thereof; where the nucleic acid molecule does not consist of the sequence shown in SEQ ID NO: 1, 18, 20 or 34.

17. A protein encoded by the nucleic acid molecule according to claim 16, where the protein does not consist of the sequence shown in SEQ ID NO: 2, 19, 21 or 35.

18. A double-stranded RNA nucleic acid molecule (dsRNA molecule) where the sense strand of said dsRNA molecule displays at least a homology of 30% to the nucleic acid molecule according to claim 16, or comprises a fragment of at least 50 base pairs, which possesses at least a 50% homology to the nucleic acid molecule according to claim 16.

19. The dsRNA molecule according to claim 18, wherein the two RNA strands are covalently bound to one another.

20. A DNA expression cassette comprising a nucleic acid sequence which is essentially identical to a nucleic acid molecule according to claim 16, where said nucleic acid sequence is present in sense orientation to a promoter.

21. A DNA expression cassette comprising a nucleic acid sequence which is essentially identical to a nucleic acid molecule according to claim 16, where said nucleic acid sequence is present in antisense orientation to a promoter.

22. A DNA expression cassette comprising a nucleic acid sequence coding for a dsRNA molecule according to claim 18, where said nucleic acid sequence is linked with a promoter.

23. The DNA expression cassette according to claim 22, where the nucleic acid sequence to be expressed is linked with a promoter functional in plants.

24. The DNA expression cassette according to claim 23, where the promoter functional in plants is a pathogen-inducible promoter.

25. A vector comprising an expression cassette according to claim 20.

26. A transgenic cell comprising a nucleic acid sequence according to claim 16.

27. A monocotyledonous organism comprising a nucleic acid sequence according to claim 16, which comprises a mutation which causes a decrease in the activity of a protein encoded by the nucleic acid molecules according to claim 16 in the organism or parts thereof.

28. A transgenic monocotyledonous organism comprising a nucleic acid sequence according to claim 16.

29. The organism according to claim 28, which has an increased Bax inhibitor 1 activity.

30. The organism according to claim 29, which has an increased Bax inhibitor 1 activity in mesophyllic cells and/or root cells.

31. The organism according to claim 28, wherein the organism belongs to the Poaceae plant family.

32. The organism according to claim 31, wherein the organism is selected from the plant genera Hordeum, Avena, Secale, Triticum, Sorghum, Zea, Saccharum, or Oryza.

33. The organism according to claim 32, wherein the organism is selected from the species Hordeum vulgare (barley), Triticum aestivum (wheat), Triticum aestivum subsp. spelta (spelt), Triticale, Avena sative (oats), Secale cereale (rye), Sorghum bicolor (millet), Zea mays (maize), Saccharum officinarum (sugar cane) and cane), or Oryza sative (rice).

34. A method for the production of a plant, or an organ, tissue or a cell thereof resistant against mesophyllic tissue-penetrating pathogens comprising transforming a plant, or an organ, tissue, or cell thereof with the nucleic acid sequence of claim 16, wherein the transformed plant, or organ, tissue, or cell thereof exhibit reduced penetration of a mesophyllic tissue-penetrating pathogen compared to an untransformed plant, or organ, tissue, or cell thereof.

35. A crop or reproductive material containing the nucleic acid sequence according to claim 16.
